SEQUENCE LISTING (12 sequences)

SEQ ID NO: 1 - DNA, 18 bp, Artificial Sequence
  Description of Artificial Sequence Synthetic primer
tggcctttggctcaatac

SEQ ID NO: 2 - DNA, 19 bp, Artificial Sequence
  Description of Artificial Sequence Synthetic primer
ctaccgctgtgaagtgatg

SEQ ID NO: 3 - DNA, 21 bp, Artificial Sequence
  Description of Artificial Sequence Synthetic primer
caagaacagcaacgagtaccg

SEQ ID NO: 4 - DNA, 20 bp, Artificial Sequence
  Description of Artificial Sequence Synthetic primer
tgacctccacagttgacagg

SEQ ID NO: 5 - DNA, 22 bp, Artificial Sequence
  Description of Artificial Sequence Synthetic primer
gcatcccagcattaggaattca

SEQ ID NO: 6 - DNA, 22 bp, Artificial Sequence
  Description of Artificial Sequence Synthetic primer
tcctcctgagcgcaagtactct

SEQ ID NO: 7 - DNA, 20 bp, Artificial Sequence
  Description of Artificial Sequence Synthetic primer
ggcatccatagaaggcatag

SEQ ID NO: 8 - DNA, 20 bp, Artificial Sequence
  Description of Artificial Sequence Synthetic primer
ggtgtagcgtgtggaaatag

SEQ ID NO: 9 - DNA, 21 bp, Artificial Sequence
  Description of Artificial Sequence Synthetic primer
gtcactggtcaactccagcac

SEQ ID NO: 10 - DNA, 21 bp, Artificial Sequence
  Description of Artificial Sequence Synthetic primer
atcaggcactccacatcttgg

SEQ ID NO: 11 - DNA, 22 bp, Artificial Sequence
  Description of Artificial Sequence Synthetic primer
ggtgagagctgcattggaggta

SEQ ID NO: 12 - DNA, 22 bp, Artificial Sequence
  Description of Artificial Sequence Synthetic primer
cggactcatcgtactcctgctt

(19) United States
(12) Patent Application Publication
(10) Pub. No.: US 2023/0285292 A1
(43) Pub. Date: Sep. 14, 2023

(54) COMPOSITIONS AND METHODS OF USING A PLA2-RESPONSIVE DRUG DELIVERY SYSTEM

(71) Applicant: THE TRUSTEES OF THE UNIVERSITY OF PENNSYLVANIA, Philadelphia, PA (US)

(72) Inventors: Zhiliang CHENG, Newtown Square, PA (US); Ling Q. QIN, Moorestown, NJ (US); Andrew B. TSOURKAS, Bryn Mawr, PA (US); Yulong WEI, Hubei (CN); Beth A. WINKELSTEIN, Philadelphia, PA (US); Sonia KARTHA, Cambridge, MA (US)

(21) Appl. No.: 18/002,681

(22) PCT Filed: Jun. 23, 2021
(86) PCT No.: PCT/US2021/038761

§ 371 Date: Dec. 21, 2022

Related U.S. Application Data
(60) Provisional application No. 63/043,025, filed on Jun. 23, 2020.
(60) Provisional application No. 63/105,573, filed on Oct. 26, 2020.

(51) Int. Cl. A61K 9/127; A61K 49/18; A61K 31/688; A61K 47/42; A61P 25/02; A61P 19/02

(52) U.S. Cl. CPC A61K 9/127; A61K 49/1839; A61K 31/688; A61K 49/1809; A61K 47/42; A61P 25/02; A61P 19/02

(57) ABSTRACT

Provided herein are compositions comprising a drug delivery system comprising a phospholipid that is hydrolyzed by phospholipase A2 (PLA2) enzyme and a drug. Also provided herein are methods for treating or for determining the location of a region to be treated or monitored in a subject in need thereof, the methods comprising: administering to the subject the disclosed composition.

### CROSS-REFERENCE TO RELATED APPLICATIONS

This application claims priority to and the benefit of U.S. Provisional Patent Application No. 63/043,025 (filed Jun. 23, 2020), and U.S. Provisional Patent Application No. 63/105,573 (filed Oct. 26, 2020), the disclosures of which are incorporated herein by reference in their entireties for any and all purposes.

### GOVERNMENT RIGHTS

This invention was made with government support under AR066098 and NS100892 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 21, 2021, is named “103241_006740_SL.txt” and is 3,014 bytes in size.

### TECHNICAL FIELD

The disclosed inventions relate to compositions and methods for treating neuropathic pain, inflammation or osteoarthritis. More particularly, the disclosed inventions relate to the field of treating a neuropathic pain, an inflammatory disease, osteoarthritis or condition in a subject using a PLA2-responsive drug delivery system.

### BACKGROUND

Neuropathic pain, which develops from direct or secondary neural tissue injury or damage, occurs in approximately 10% of the US population and often persists for several years or even a lifetime after an initial injury.

In addition to the primary injury to neural tissue that occurs with nerve root trauma and spinal cord injury, painful neuropathic injuries are accompanied by a robust secondary neuroinflammatory response, both at the site of injury and remote to it in the spinal cord. Many different animal models of neuropathic pain have defined a host of inflammatory cascades both at the site of injury and in the central nervous system (CNS), contributing to central sensitization that is responsible for persistent pain. During central sensitization there is increased neuronal hyperexcitability and spontaneous activity in spinal neurons, which can result from and be exacerbated by, inflammatory mediators that directly act on neurons. Among the potent inflammatory mediators involved in spinal neuroimmune regulation of pain is the family of phospholipase-A₂ (PLA₂) enzymes. These enzymes recognize and catalytically hydrolyze the sn-2 ester bond of glycerophospholipids, releasing free fatty acids, such as arachidonic acid (AA) and lysophospholipids, that are involved in tissue damage and neuronal injury, as well as a host of inflammatory and neurological disorders. Many studies have reported expression of the secretory PLA₂ (sPLA₂), an isoform of the PLA₂ enzyme, to be associated with a number of painful pathologies characterized by inflammation, such as disc herniation, disc degeneration, discogenic pain and neuropathic pain. sPLA₂ is normally present in the mammalian spinal cord and brain. Under pathological conditions sPLA₂ can be induced by multiple cascades and effector molecules including inflammatory cytokines, free radicals, and excitatory amino acids. Elevated sPLA₂ expression after neuropathic injury is observed both centrally in the spinal cord and in the peripheral dorsal root ganglion (DRG), both anatomic sites that contribute to the potentiation and amplification of chronic pain after trauma.

Despite its prevalence, neuropathic pain remains largely resistant to treatment, leading to chronic disability, with staggering societal and economic costs. Current neuropathic pain treatments include surgical intervention and pharmacologic approaches, but are largely ineffective, providing only transient pain relief. Although treatment of neuropathic pain through opioid and non-opioid analgesics has been extensively pursued, these approaches are often accompanied by undesired side effects including addiction and increased pharmacological tolerance. Clinical management of neuropathic pain is further complicated by lack of effective drug delivery. Systemic delivery of small molecule drugs only provides a small portion of medication at the site of injury, and even less drug is delivered to the spinal cord where pain modulation occurs.

Osteoarthritis (OA) is a painful and debilitating disease of articular cartilage, leading to joint pain, loss of joint function, and deleterious effects on the quality of daily life. It occurs in approximately 27 million adults in the United States alone, with staggering societal and economic costs (60 billion/year)(1). Current treatments for OA include non-pharmacological treatment (i.e. diet and exercise), pharmacological approaches and surgical intervention. Although many pharmacologic approaches have been extensively pursued and some drugs have shown promise in preclinical studies, none has emerged with any significant clinical success, and there are no disease-modifying therapies available to delay and/or limit OA development and progression.

Among the potent inflammatory mediators involved in the development of OA is the family of secreted phospholipase A2 (sPLA2) enzymes. sPLA2 is a heterogeneous group of enzymes that specifically recognize and catalytically hydrolyze the sn-2 ester bond of glycerophospholipids, releasing free fatty acids such as arachidonic acid (AA) and lysophospholipids, that are well-known mediators of inflammation and tissue damage. sPLA2 is normally present at low levels in healthy knee joint tissues. Yet, under pathological conditions sPLA2 can be induced by multiple cascades and effector molecules including inflammatory cytokines and free radicals. A high expression level and activity of sPLA2 is observed in the synovial membrane, synovial fluid, and articular cartilage of human OA patients. As such, if sPLA2 is a sensitive and unique marker for OA-related inflammation, it can be postulated that inhibition of sPLA2 enzyme activity by sPLA2i can be exploited to act as a novel therapeutic strategy for OA treatment. Note that, several sPLA2i compounds have already been developed and used in clinical trials for treating other inflammatory diseases. However, few studies have examined the role of sPLA2 in OA and no study has sought to harness the sPLA2 activity for the treatment of OA.

Thus, there is a need in the art to develop new interventional platforms for greater effectiveness in treating radiculopathy, neuropathy and OA. This invention addresses this need.

### SUMMARY

In meeting the described long-felt needs in the art, first provided herein are compositions comprising: a drug delivery system comprising a drug and a phospholipid that is hydrolyzed by phospholipase A2 (PLA2) enzyme. In some embodiments, the phospholipid, when hydrolyzed, allows release of the drug from the drug delivery system.

Therapeutic, prophylactic, or diagnostic drugs can be conjugated onto phospholipid via the sPLA2-cleavable sn-2 ester bond. To control the selective release of the drug by sPLA2, aminocaproic spacers between the drug and lipid backbone can be used. This synthesis method is unique, and therapeutic, prophylactic, or diagnostic drugs can be conjugated onto lipids using this synthesis method and then incorporated into the drug delivery system, including liposomes or micelles.

Also provided herein are pharmaceutical compositions comprising the drug delivery system disclosed herein and a pharmaceutically acceptable carrier. In some embodiments, the compositions disclosed herein are used for medicaments and for the preparation of medicaments for the treatment of diseases or conditions associated with inflammation.

Also provided herein are methods for treating a subject in need thereof. The methods comprise administering to the subject a drug delivery disclosed herein.

Further provided herein are methods for determining the location of a region to be treated or monitored in a patient in need thereof. The methods comprise administering to a subject a composition comprising a phospholipid that is hydrolyzed by phospholipase A2 (PLA2) enzyme, optionally comprising a drug; and determining a location, within the subject, of any component of the composition.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1B are series of schematic diagrams illustrating the presently disclosed PLA2-responsive drug delivery system. (FIG. 1A) Schematic diagram of PLA2 responsive micelles. Multifunctional micelles were formed through the coassembly of the phospholipid and the small hydrophobic SPIO nanoparticles. PLA2 inhibitor was incorporated into the phospholipid membrane during the micelle preparation. (FIG. 1B) Nerve root compression injury was performed using a 10 gf microvascular clip applied (15 min) to the C7 dorsal nerve root. PLA₂ responsive micelles were injected directly onto the nerve root ipsilateral to injury or intravenous injection into the tail.

FIGS. 2A-2B are series of images and histograms demonstrating that spinal sPLA₂ expression increases only on the side ipsilateral to injury after a painful compression and is further elevated with time after injury. (FIG. 2A) Representative images of the ipsilateral spinal dorsal horn show increased sPLA₂ immunoreactivity in all groups at day 7 compared to expression at day 1, with greatest expression in the painful group at each time point. The scale bar is 200 μm and applies to all panels. (FIG. 2B) sPLA₂ expression in the painful group is significantly greater than expression for both the nonpainful compression and sham groups on both days 1 (*p<0.048) and 7 (**p<0.009). Expression in the painful group at day 7 is significantly increased from levels at day 1 (#p<0.043).

FIGS. 3A-3D are series of images and histograms demonstrating that the expression of sPLA₂ in the ipsilateral dorsal horn varies with cell type and over time. (FIG. 3A) Representative images show the relative co-localization (arrows) for neuronal, microglial and astrocytic sPLA₂ at days 1 and 7. The scale bar is 100 μm and applies to images. (FIG. 3B) Neuronal sPLA₂ is significantly elevated after painful compression on both day 1 (*p<0.0001) and day 7 (**p<0.04), with neuronal sPLA₂ expression significantly decreasing at day 7 after painful injury as compared to levels at day 1 (#p=0.0191). (FIG. 3C) Similarly, microglial sPLA₂ increases over both nonpainful compression and sham levels in the painful group on both day 1 (*p<0.01) and day 7 (**p<0.001) and is significantly increased at day 7 from levels at day 1 (#p=0.0002) in the painful group. (FIG. 3D) Astrocytic sPLA₂ increases after painful injury only at day 7 over both nonpainful (**p=0.009) and sham control groups (**p=0.029), with expression significantly greater than comparable painful levels at day 1 (#p=0.036).

FIGS. 4A-4D are series of images and graphs depicting the transmission electron microscopy (TEM) image of individual hydrophobic SPIO (FIG. 4A) and PLA₂ inhibitor and SPIO-loaded phospholipid micelle (FIG. 4B). (FIG. 4C) Number-weighted size distribution of PLA₂ inhibitor and SPIO-loaded phospholipid micelles by dynamic light scattering (DLS). (FIG. 4D). Relaxivity determination for PLA₂ inhibitor and SPIO-loaded phospholipid micelles.

FIG. 5 is a graph depicting in vitro response of PLA₂ inhibitor and SPIO-loaded micelles. 10 μL, PLA₂ inhibitor and SPIO-loaded micelles ([PLA₂ inhibitor]=0.25 mg/mL) were mixed with 6.67 μL, PLA₂ enzyme ([PLA₂]=7.5 U/mL) and incubated for 20 minutes at room temperature before adding to NBD-incorporated liposomal suspension. Measurements of the PLA₂ induced dequenching of NBD trapped within the liposomes were carried out as follows: 20 IA of NBD-incorporated liposomal suspension ([HSPC]=1 mg/mL) was added to 0.48 mL of 0.01M HEPES (pH 7.4) buffer solution containing 2 mM CaCl₂). After 5 minutes, the preincubation mixture of PLA₂ inhibitor-loaded micelles and PLA₂ enzyme was added to NBD-incorporated liposomal suspension. The fluorescence intensity at 520 nm was measured using an excitation at 460 nm. 20 μL, Triton X-100 (50 mM) was added to the suspension at the end of experiment. A similar approach was used to study the PLA₂ response of control micelles (i.e., without PLA₂-inhibitor).

FIGS. 6A-6B are series of histograms showing that the incubation of PLA₂ inhibitor and SPIO-loaded phospholipid micelles with dorsal root ganglion cells was not cytotoxic with any loaded concentration of sPLA₂-inhibitor. (FIG. 6A) Cell viability in neuronal cultures was unchanged from control (0 μg/mL) following incubation with any micelle concentration. (FIG. 6B) sPLA₂-inhibitor-loaded micelles does not significantly increase the average percent of cell lysis observed in primary DRG cultures, with no significant differences for any micelle concentrations compared to control.

FIGS. 7A-7B are series of graphs demonstrating that local administration of PLA₂ inhibitor and SPIO-loaded phospholipid micelles immediately after injury prevents the development of pain that typically develops after painful nerve root compression for up to 7 days. (FIG. 7A) Ipsilateral paw withdrawal thresholds following treatment with PLA₂ inhibitor and SPIO-loaded phospholipid micelles were significantly higher (#p<0.016) than thresholds following treatments with micelles alone. Administration of control micelles did not prevent pain with withdrawal thresholds significantly lower than pre-injury levels (day 0) that lasts for 7 days (*p<0.0001). (FIG. 7B) Withdrawal threshold in the contralateral paw were not significantly different between groups on any day tested.

FIGS. 8A-8B are series of graphs demonstrating that repeated intravenous (IV) administration of PLA₂ inhibitor and SPIO-loaded phospholipid micelles on days 1 and 2 after nerve root compression abolishes pain for up to 7 days. (FIG. 8A) A 15 min nerve root compression significantly reduces withdrawal thresholds in the ipsilateral forepaw as early as day 1 compared to sham (*p=0.001) and baseline (\hat{ }p=0.0053) and responses remain significantly lower following repeated administration on days 1 and 2 of control micelles (without PLA₂-inhibitor), for up to 7 days after the initial injury compared to the sham surgical control group (\hat{ }p<0.002) withdrawal thresholds. Similarly, prior to treatment with PLA₂ inhibitor and SPIO-loaded phospholipid micelles, thresholds on day 1 are significantly lower than those of the pre-injury response on day 0 (#p=0.0001) and sham responses on day 1 (**p=0.0004). However, following micelle administration on day 1, withdrawal thresholds on day 2 are significantly increased back to pre-injury levels and sham response levels. Withdrawal thresholds following the second micelle treatment on day 2, remain elevated through day 7 and are significantly increased (*p<0.024) over comparable responses in the group that received nerve root compression only. (FIG. 8B) There were no significant differences in the contralateral withdrawal thresholds for any of the groups assessed.

FIG. 9 is a series of images showing that PLA₂ inhibitor and SPIO-loaded phospholipid micelles localized to the injured nerve root 7 days after local injection. Iron was detected only in the ipsilateral C7 dorsal nerve root after a 15 min NRC treated with micelles. There was no evidence of iron in the contralateral C7 nerve root after micellar treatment or both dorsal nerve roots that received a 15 min NRC only. The scale bar is 100 μm and applies to all panels

FIG. 10 is a series of images showing that intravenously administered PLA₂ inhibitor and SPIO-loaded phospholipid micelles localized to the injured nerve root at day 7. Iron was detected in C7 dorsal nerve root only after a 15 min NRC treated with micelles. There was no evidence of iron in sham-operated and naïve roots. Scale bar is 200 μm and applies to all panels.

FIG. 11 is a series of images and graphs showing that administration of PLA₂ inhibitor-loaded micelles immediately after a 15 min NRC reduces spinal sPLA₂ expression 7 days after injury. sPLA₂ expression is significantly decreased (*p<0.0001) following treatment with the micelles with inhibitor compared to treatment with control micelles only. Scale bar 100 μm and applies to all panels.

FIG. 12 is a series of images and graphs showing that spinal Iba1 expression is decreased 7 days after treatment with of PLA₂ inhibitor-loaded micelles. Iba1 expression in the superficial dorsal horn is significantly decreased (*p=0.0002) after treatment of micelles with inhibitor compared to treatment with control micelles alone. Scale bar 100 μm and applies to all panels.

FIG. 13 is a series of images and graphs showing that spinal sPLA₂ expression is significantly decreased 7 days after intravenous administration of PLA₂ inhibitor-loaded micelles. Spinal sPLA₂ expression is significantly decreased (*p=0.038) in the superficial dorsal horn after treatment of micelles with inhibitor compared to treatment with control micelles alone. Scale bar 100 μm and applies to all panels.

FIGS. 14A-14C are series of graphs and a schematic diagrams illustrating the response of PLA₂-inhibitor loaded micelles (FIG. 14A) In vitro response of PLA₂-inhibitor loaded micelles (i.e., no SPIO). 10 μL PLA₂ inhibitor-loaded micelles ([PLA₂ inhibitor]=0.25 mg/mL) were mixed with 6.67 μL PLA₂ enzyme ([PLA₂]=7.5 U/mL) and incubated for 20 minutes at room temperature before adding to NBD-incorporated liposomal suspension. Measurements of the PLA₂ induced dequenching of NBD trapped within the liposomes were carried out as follows: 20 μL of NBD-incorporated liposomal suspension ([HSPC]=1 mg/mL) was added to 0.48 mL of 0.01M HEPES (pH 7.4) buffer solution containing 2 mM CaCl₂. After 5 minutes, the preincubation mixture of PLA₂ inhibitor-loaded micelles and PLA₂ enzyme was added to NBD-incorporated liposomal suspension. The fluorescence intensity at 520 nm was measured using an excitation at 460 nm. 20 μL Triton X-100 (50 mM) was added to the suspension at the end of experiment. A similar approach was used to study the PLA₂ response of control micelles (i.e., without PLA₂ inhibitor). All experiments were performed at room temperature. (FIG. 14B) Number-weighted size distribution of PLA₂-inhibitor-loaded micelles by dynamic light scattering (DLS). (FIG. 14C) PLA₂ inhibitor-loaded micelles (i.e., no SPIO).

FIGS. 15A-15C are series of graphs and a schematic diagrams illustrating the response of PLA₂-inhibitor loaded liposomes. (FIG. 15A) In vitro response of PLA₂-inhibitor loaded liposomes. 10 μL PLA₂ inhibitor-loaded liposomes ([PLA₂ inhibitor]=0.25 mg/mL) were mixed with 6.67 μL PLA₂ enzyme ([PLA₂]=7.5 U/mL) and incubated for 20 minutes at room temperature before adding to NBD-incorporated liposomal suspension. Measurements of the PLA₂ induced dequenching of NBD trapped within the liposomes were carried out as follows: 20 μL of NBD-incorporated liposomal suspension ([HSPC]=1 mg/mL) was added to 0.48 mL of 0.01M HEPES (pH 7.4) buffer solution containing 2 mM CaCl₂). After 5 minutes, the preincubation mixture of PLA₂ inhibitor-loaded liposomes and PLA₂ enzyme was added to NBD-incorporated liposomal suspension. The fluorescence intensity at 520 nm was measured using an excitation at 460 nm. 20 μL Triton X-100 (50 mM) was added to the suspension at the end of experiment. A similar approach was used to study the PLA₂ response of control liposomes (i.e., without PLA₂-inhibitor). All experiments were performed at room temperature. (FIG. 15B) Number-weighted size distribution of PLA₂-inhibitor-loaded liposomes by dynamic light scattering (DLS). (FIG. 15C) PLA₂ inhibitor-loaded liposomes.

FIGS. 16A-16D: sPLA₂ expression in human and mouse OA cartilage. FIG. 16A—Representative images of safranin 0/fast green staining (top) and immunohistochemistry of sPLA₂ (bottom) in healthy juvenile, healthy adult, early stage, middle stage and late stage OA human cartilage tissues. Scale bars, 200 μm. FIG. 16B—Quantification of sPLA₂-positive chondrocytes as a proportion of total chondrocytes in healthy and OA human articular cartilage tissues. (n=5). FIG. 16C—Representative images of safranin 0/fast green staining (top) and immunohistochemistry of sPLA₂ (bottom) in tibial articular cartilage of WT mice at 1 month post-sham or post-DMM surgery. Scale bars, 50 μm. FIG. 16D—Quantification of sPLA₂-positive chondrocytes in tibial articular cartilage of sham- or DMM-operated joints (n=5). Statistical analysis was performed using one-way ANOVA with Dunnett's post hoc test for FIG. 16B and paired two-tailed t-test for FIG. 16D. Data presented as mean±s.e.m. ***p<0.001.

FIGS. 17A-17F: Preparation and characterization of sPLA2i-NPs. FIG. 17A—Schematic diagram of sPLA2i-NPs, in which a lipid-based sPLA2i was incorporated into nanometer-sized phospholipid nanoparticles (DSPE-PEG2000) with cationic lipid DOTAP doped. FIG. 17B—DLS measurement of sPLA2i-NPs hydrodynamic diameter and TEM (insert) of sPLA2i-NPs. FIG. 17C—Zeta potential of sPLA2i-NPs in the presence or absence of cationic lipid DOTAP in 0.1 M PBS (pH=7.4). FIG. 17D—The stability of sPLA2i-NPs in water was accessed by monitoring the hydrodynamic diameter for up to 1 week. FIG. 17E—In vitro response of sPLA2i-NPs to sPLA2 enzyme experiment showed a significant inhibition effect. Graph lines represent adding Ctrl-NP+sPLA2 enzyme (top line); sPLA2 enzyme (middle line); and sPLA2i-NP+sPLA2 enzyme (bottom line). FIG. 17F—The cytotoxicity of sPLA2i-NPs was determined by measuring the cell viability of primary chondrocytes after co-incubation with sPLA2i-NPs at various concentrations. In all datasets, n=3 biologically independent experiments. Statistical analysis was performed using one-way ANOVA with Dunnett's post hoc test. Data presented as mean±s.e.m.

FIGS. 18A-18F: Cartilage penetration and joint retention of sPLA2i-NPs. FIG. 18A—Representative confocal microscope images of cross-sections of bovine cartilage explants incubated with free rhodamine dye or rhodamine-labeled sPLA2i-NPs in the presence or absence of cationic lipid DOTAP for 0, 2, 4, 6 and 8 days. Scale bar, 200 μm. FIG. 18B—Quantitative analysis of the fluorescence intensity of the above different formulations over the entire explant sections (n=3). Graph lines represent sPLA2i-NP (DOTAP+, top line); Rhod-PE (middle line); and sPLA2i-NP (DOTAP−, bottom line). FIG. 18C—Quantification of the area under the curve (AUC) based on the fluorescence intensity profiles in b (n=3). FIG. 18D—Representative IVIS images of healthy and OA (OA was induced surgically 8 weeks before) mouse knee joints over 28 days post single intra-articular injection of free ICG or Cy7-labeled sPLA2i-NPs. Fluorescent scale, min=2.0×10⁷, max=6.0×10⁷. FIG. 18E—Quantitative analysis of time course fluorescent radiant efficiency within healthy and OA mouse knee joints over 28 days (n=5). Graph lines represent OA+sPLA2i-NP (top line); sPLA2i-NP (second line from the top); OA+ICG (third line from the top); ICG (bottom line). FIG. 18F—Quantification of the area under the curve (AUC) based on the fluorescence intensity profiles in FIG. 18E (n=5). Rhod-PE: free rhodamine dye, sPLA2i-NPs in the absence of DOTAP: sPLA2i-NPs (DOTAP−), sPLA2i-NPs in the presence of DOTAP: sPLA2i-NPs (DOTAP+). Statistical analysis was performed using one-way ANOVA with Turkey's post hoc test. Data presented as mean±s.e.m. ***p<0.001.

FIGS. 19A-19G: Penetration and chondroprotective effects of sPLA2i-NPs in mouse femoral heads. FIG. 19A—Representative fluorescence images of cross-sections of mouse femoral heads stimulated with recombinant mouse IL-1β and incubated with rhodamine-labeled sPLA2i-NPs for 0, 24 or 48 hours. Magnified images of the white boxed areas are presented as bottom panels. Scale bars, 100 μm. FIG. 19B—Quantitative analysis of rhodamine-labeled sPLA2i-NPs penetration depth into IL-1β-stimulated mouse femoral heads (n=6). Graph lines represent data at 48 h (top line); and data at 24 h (bottom line). FIG. 19C—Quantification of the area under the curve (AUC) based on the fluorescence intensity profiles in b (n=6). FIG. 19D—Representative images of safranin-O/fast green staining on the sections of untreated and PBS-, Ctrl-NP- and sPLA2i-NP-treated IL-1β-stimulated mouse femoral heads. Magnified images of the black boxed areas are presented as bottom panels. Scale bars, 100 μm. FIG. 19E—Safranin-O-positive area among the above groups was quantified (n=5). FIG. 19F—The OA severity was accessed by Mankin score (n=5). FIG. 19G—The relative gene expression of sPLA2-IIA, Aggrecan, Col2a1, Mmp13 and Adamts5 was examined by qRT-PCR in the untreated and PBS-, Ctrl-NP- and sPLA2i-NP-treated IL-1β-stimulated mouse femoral heads (n=3). Statistical analysis was performed using paired two-tailed t-test for FIG. 19C and one-way ANOVA with Turkey's post hoc test for FIGS. 19E, 19F, and 19G. Data presented as mean±s.e.m. *p<0.05, **p<0.01, ***p<0.001.

FIGS. 20A-20L: Therapeutic effects of sPLA2i-NPs for attenuation of DMM-induced traumatic OA. FIG. 20A—The study design of sPLA2i-NP treatment for DMM-induced OA mice. DMM surgery was performed on 3-month-old male mice followed by intra-articular injections of PBS, sPLA2i, Ctrl-NPs and sPLA2i-NPs once every week for 2 or 4 months. FIG. 20B—Representative images of safranin 0/fast green staining in the articular cartilage of sham- or DMM-operated knee joints with 2- or 4-month treatment. Scale bar, 200 μm. FIG. 20C—The OA severity was accessed by Mankin score after 2-month treatment (n=8). The bars in FIGS. 20C, 20E, 20G, and 20I are arranged in the following order from left to right: Sham, DMM PBS, DMM sPLA2i, DMM Ctrl-NP, and DMM sPLA2i-NP. FIG. 20D—Representative images of H&E staining in the sham- or DMM-operated knee joints with 2-month treatment. Scale bar, 100 μm. Red boxed area indicate the enlargement of synovial lining cell layer. FIG. 20E—Synovial inflammation was evaluated by synovitis score after 2-month treatment (n=8). FIG. 20F—The subchondral bone plate (SBP) thickness of sham- or DMM-operated knee joints with 4-month treatment was revealed by representative 3D color maps. Color ranges from 0 (blue) to 320 μm (red). FIG. 20G—Quantitative analysis of the SBP thickness at the posterior site of femoral medial condyle after 4-month treatment (n=8). FIG. 20H—Osteophytes on the sham- or DMM-operated knee joints with 4-month treatment was revealed by representative 2D (top) and 3D (bottom) microCT images. Red arrows indicate the osteophytes. FIG. 20I—Quantitative analysis of the total osteophyte volume after 4-month treatment (n=8). FIG. 20J—von Frey assay on sham- or DMM-operated knee joints with 4-month treatment was performed at 1, 2, 4, 8, 12 weeks post-surgery (n=8). The data of day 0 was acquired before DMM surgery. Graph lines represent: Sham (top line); sPLA2i-NP (second line from the top); sPLA2i (third line from the top); PBS (fourth line from the top); Ctrl-NP (bottom line). FIG. 20K—Representative images of immunohistochemistry staining of sPLA2-IIa, p-P65 and p-P100 in the tibial articular cartilage from sham- or DMM-operated knee joints with 2-month treatment. Scale bar, 100 μm. FIG. 20L—Quantification of sPLA2-IIa-, p-P65- and p-P100-positive chondrocytes in the tibial articular cartilage after 2-month treatment (n=5). The bars in FIG. 20L are arranged in the following order from left to right: Sham, DMM PBS, DMM sPLA2i, DMM Ctrl-NP, and DMM sPLA2i-NP. Statistical analysis was performed using one-way ANOVA with Turkey's post hoc test. Data presented as mean±s.e.m. *p<0.05, **p<0.01, ***p<0.001 in FIGS. 20C, 20E, 20G, 20I, and 20L. *p<0.05, **p<0.01 for sPLA2i-NPs vs. PBS in FIG. 20J.

FIGS. 21A-21J: Therapeutic effects of sPLA2i-NPs for attenuation of load-induced post-traumatic OA. FIG. 21A—The study design of sPLA2i-NP treatment for loading-induced OA mice. 6N- or 9N-loading was performed on 3-month-old male mice. Intra-articular injections of PBS, sPLA2i, Ctrl-NPs and sPLA2i-NPs were made immediately and 48 hours post loading. FIG. 21B—Representative images of safranin 0/fast green staining in the articular cartilage of sham- or load (6N or 9N)-operated knee joints with 14-day treatment. Scale bar, 100 μm. Dashed line indicate the range of loss of staining. FIG. 21C—Quantitative analysis of the length of the cartilage lesion range in the sham- or 6N-load-operated knee joints with 14-day treatment. The bars in FIGS. 20C, 20E, and 20G are arranged in the following order from left to right: Sham, 6N PBS, 6N sPLA2i, 6N Ctrl-NP, and 6N sPLA2i-NP. FIG. 21D—Representative images of TUNEL staining in the tibial articular cartilage from sham- and 6N-load-operated knee joints with 14-day treatment. Scale bar, 100 μm. FIG. 21E—Quantification of TUNEL-positive chondrocytes in the tibial articular cartilage after 14-day treatment (n=5). FIG. 21F—Representative images of immunohistochemistry staining of sPLA2-IIa, p-P65 and p-P100 in the tibial articular cartilage from sham- and 6N-load-operated knee joints with 14-day treatment. Scale bar, 100 μm. FIG. 21G—Quantification of sPLA2-IIa-, p-P65- and p-P100-positive chondrocytes in the tibial articular cartilage after 14-day treatment (n=5). FIG. 21H—Representative images of H&E staining in the sham- and 6N-load-operated knee joints with 14-day treatment. Scale bar, 100 μm. Red boxed area indicate the enlargement of synovial lining cell layer. FIG. 21I—Synovial inflammation was evaluated by synovitis score after 14-day treatment (n=8). The bars in FIG. 20I are arranged in the following order from left to right: Sham, 6N PBS, 6N sPLA2i, 6N Ctrl-NP, and 6N sPLA2i-NP. FIG. 21J—von Frey assay on sham and 6N-load-operated knee joints with 14-day treatment at 1 or 2 weeks post loading (n=8). Graph lines represent: Sham (top line at 2 weeks); sPLA2i-NP (second line from the top at 2 weeks); Ctrl-NP (third line from the top at 2 weeks); PBS (fourth line from the top at 2 weeks); sPLA2i (bottom line at 2 weeks). The data of day 0 was acquired before loading. Statistical analysis was performed using one-way ANOVA with Turkey's post hoc test. Data presented as mean s.e.m. *p<0.05, ***p<0.001 in FIGS. 21C, 21E, 21G, and 21I. *p<0.05 for sPLA2i-NPs vs. PBS in FIG. 21J.

FIG. 22: The stability of sPLA2i-NPs in synovial fluid. The stability of sPLA2i-NPs in synovial fluid was accessed by monitoring the hydrodynamic size (diameter) for up to 24 hours.

FIG. 23: Quenching study of NBD-liposomes (20 mol % NBD-PC/80 mol % HSPC liposome). Top line—NBD liposomes with Triton; bottom line—NBD-liposomes.

FIGS. 24A-24F: Penetration ability of sPLA2i-NPs. FIGS. 24A, 24C, and 24E—Quantitative analysis of free rhodamine dye (FIG. 24A), rhodamine-labeled sPLA2i-NPs (DOTAP−) (FIG. 24C), and sPLA2i-NPs (DOTAP+) (FIG. 24E) penetration depth into bovine cartilage explants over 8-day incubation (n=3). Graph lines in FIGS. 24A, 24C, and 24E represent: D8 (top line); D6 (second line from the top); D4 (third line from the top); D2 (bottom line). FIGS. 24B, 24D, and 24F—Quantitative analysis of area under the curve (AUC) based on the corresponding fluorescence intensity profiles in FIGS. 24A, 24C, and 24E, respectively (n=3). Rhod-PE: free rhodamine dye, sPLA2i-NPs in the absence of DOTAP: sPLA2i-NPs (DOTAP−), sPLA2i-NPs in the presence of DOTAP: sPLA2i-NPs (DOTAP+). Statistical analysis was performed using one-way ANOVA with Dunnett's post hoc test. Data presented as mean±s.e.m. **p<0.01, ***p<0.001.

FIGS. 25A and 25B: sPLA2i-NP uptake in bovine cartilage explants. FIG. 25A—Representative IVIS images of bovine cartilage explants incubated with free rhodamine dye, rhodamine-labeled sPLA2i-NPs (DOTAP−), and sPLA2i-NPs(DOTAP+) for 24 hours. Fluorescent scale, min=4.87×10⁷, max=9.08×10⁸. FIG. 25B—Quantitative analysis of fluorescent radiant efficiency on the surfaces of bovine cartilage explants after 24-hour incubation (n=8). Rhod-PE: free rhodamine dye, sPLA2i-NPs in the absence of DOTAP: sPLA2i-NPs (DOTAP−), sPLA2i-NPs in the presence of DOTAP: sPLA2i-NPs (DOTAP+). Statistical analysis was performed using one-way ANOVA with Turkey's post hoc test. Data presented as mean±s.e.m. *p<0.05, ***p<0.001.

FIGS. 26A-26C: The retention ability of sPLA2i-NPs in rat knee joints. FIG. 26A—Representative IVIS images of healthy rat knee joints over 28 days post single intra-articular injection of Cy7-labeled sPLA2i-NPs or free ICG. Fluorescent scale, min=4.0×10⁷, max=1.0×10⁹. FIG. 26B—Quantitative analysis of time course fluorescent radiant efficiency within healthy rat knee joints over 28 days (n=3). The lines represent: sPLA2i-NP (top line) and ICG (bottom line). FIG. 26C—Quantification of the area under the curve (AUC) based on the fluorescence intensity profiles in b (n=3). Statistical analysis was performed using paired two-tailed t-test. Data presented as mean±s.e.m. ***p<0.001.

FIGS. 27A and 27B: Biodistribution of sPLA2i-NP within mice knee joint following local injection. FIG. 27A—Biodistribution of Cy7-labeled sPLA2i-NPs within healthy mouse knee joints at 24 hours post single injection of Cy7-labeled sPLA2i-NPs. Fluorescent scale: min=3.0×10⁷, max=1.0×10⁸. FIG. 27B—Quantification of fluorescent radiant efficiency in the different components of knee joints (n=4).

FIGS. 28A and 28B: Systemic biodistribution of sPLA2i-NPs following local injection into healthy mouse knee joints. FIG. 28A—Biodistribution of Cy7-labeled sPLA2i-NPs within several internal major organs and blood sample at 24 hours or 1 month post single injection of PBS or Cy7-labeled sPLA2i-NPs. Fluorescent scale: max=3.0×10⁷, min=1.0×10⁸. FIG. 28B—Quantification of fluorescent radiant efficiency within different organs and blood sample at indicated time points (n=4). The bars in FIG. 28B are arranged in the following order from left to right: PBS 24 h; sPLA2i-NP 24 h; sPLA2i-NP 1M. Statistical analysis was performed using one-way ANOVA with Turkey's post hoc test. Data presented as mean±s.e.m. **p<0.01, ***p<0.001.

FIG. 29: Effects of sPLA2i-NPs on OA progression after 4-month treatment. The OA severity was accessed by Mankin score after 4-month treatment (n=8). DMM surgery was performed on 3-month-old male mice followed by intra-articular injections of PBS, sPLA2i, Ctrl-NPs and sPLA2isPLA2i-NPs once every week for 4 months. Statistical analysis was performed using one-way ANOVA with Turkey's post hoc test. Data presented as mean±S.E.M. *p<0.05, ***p<0.001.

FIGS. 30A-30B: Effects of sPLA2i-NPs on cartilage thickness after 2- or 4-month treatment. FIG. 30A—Quantification of average thicknesses of uncalcified (Uncal. Th.) and calcified (Cal. Th.) cartilage after 2-month treatment (n=8). FIG. 30B—Quantification of average thicknesses of uncalcified (Uncal. Th.) and calcified (Cal. Th.) cartilage after 4-month treatment (n=8). DMM surgery was performed on 3-month-old male mice followed by intra-articular injections of PBS, sPLA2i, Ctrl-NPs and sPLA2i-NPs once every week for 2 or 4 months. Statistical analysis was performed using one-way ANOVA with Turkey's post hoc test. Data presented as mean±s.e.m. ***p<0.001.

FIGS. 31A-31B: Effects of sPLA2i-NPs on altering joint inflammation after 4-month treatment. FIG. 31A—Representative images of immunohistochemistry staining of sPLA2-IIa, p-P65 and p-P100 in the tibial articular cartilage from sham- and DMM-operated knee joints with 4-month treatment. Scale bar, 100 μm. FIG. 31B—Quantification of sPLA2-IIa-, p-P65- and p-P100-positive chondrocytes in the tibial articular cartilage after 4-month treatment (n=5). DMM surgery was performed on 3-month-old male mice followed by intra-articular injections of PBS, sPLA2i, Ctrl-NPs and sPLA2i-NPs once every week for 4 months. Statistical analysis was performed using one-way ANOVA with Turkey's post hoc test. Data presented as mean±s.e.m. *p<0.05, **p<0.01, ***p<0.001.

FIG. 32: Histological evaluation of systemic toxicity in vivo. Representative images of H&E staining of mouse kidney, liver, lung, heart and spleen after 2-month serial injections (once every other week) of PBS, sPLA2i, Ctrl-NPs and sPLA2i-NPs. Scale bar, 200 μm.

FIG. 33: Histological evaluation of local toxicity in vivo. Representative images of H&E staining of mouse knee joints after 2-month serial injections (once every other week) of PBS, sPLA2i, Ctrl-NPs and sPLA2i-NPs. Scale bar, 200 μm.

FIGS. 34A-34C: Serum toxicology makers after 2-month serial injections (once every other week) of PBS, sPLA2i, Ctrl-NPs and sPLA2i-NPs. FIG. 34A—Hematologic parameters: RBC counts, WBC counts, hemoglobin and platelet counts. FIG. 34B—Renal (BUN) and liver (ALT, AST, Total bilirubin) function. FIG. 34C—serum electrolytes (sodium, potassium, Chloride and Calcium) showed no significant difference among the 4 treatment groups (n=3). Statistical analysis was performed using one-way ANOVA with Turkey's post hoc test. Data presented as mean±s.e.m.

FIG. 35: Effects of sPLA2i-NPs on acute cartilage injury after 9N-loading. Quantitative analysis of the length of the cartilage lesion range in the sham- or 9N-load-operated knee joints with 14-day treatment. 9N-loading was performed on 3-month-old male mice. Intra-articular injections of PBS, sPLA2i, Ctrl-NPs and sPLA2i-NPs were made immediately and 48 hours post loading. Statistical analysis was performed using one way ANOVA with Turkey's post hoc test. Data presented as mean±s.e.m. **p<0.01, ***p<0.001.

FIGS. 36A and 36B: Effects of sPLA2i-NPs on chondrocyte survival after 9N-loading. FIG. 36A—Representative images of TUNEL staining in the tibial articular cartilage from sham- and 9N-load-operated knee joints with 14-day treatment. Scale bar, 100 μm. FIG. 36B—Quantification of TUNEL-positive chondrocytes in the tibial articular cartilage after 14-day treatment (n=5). 9N-loading was performed on 3-month-old male mice. Intra-articular injections of PBS, sPLA2i, Ctrl-NPs and sPLA2i-NPs were made immediately and 48 hours post loading. Statistical analysis was performed using one-way ANOVA with Turkey's post hoc test. Data presented as mean±s.e.m. *p<0.05, **p<0.01.

FIGS. 37A-37B: Effects of sPLA2i-NPs on altering joint inflammation after 9N-loading. FIG. 37A—Representative images of immunohistochemistry staining of sPLA2-IIa, p-P65 and p-P100 in the tibial articular cartilage from sham- and 9N-load-operated knee joints with 14-day treatment. Scale bar, 100 μm. FIG. 37B—Quantification of sPLA2-IIa-, p-P65- and p-P100-positive chondrocytes in the tibial articular cartilage after 14-day treatment (n=5). 9N-loading was performed on 3-month-old male mice. Intra-articular injections of PBS, sPLA2i, Ctrl-NPs and sPLA2i-NPs were made immediately and 48 hours post loading. Statistical analysis was performed using one-way ANOVA with Turkey's post hoc test. Data presented as mean±s.e.m. ***p<0.001.

FIGS. 38A-38B: Effects of sPLA2i-NPs on altering synovitis after 9N-loading. FIG. 38A—Representative images of H&E staining in the sham- and 9N-load-operated knee joints with 14-day treatment. Scale bar, 100 μm. Red boxed area indicate the enlargement of synovial lining cell layer. FIG. 38B—Synovial inflammation was evaluated by synovitis score after 14-day treatment (n=8). 9N-loading was performed on 3-month-old male mice. Intra-articular injections of PBS, sPLA2i, Ctrl-NPs and sPLA2i-NPs were made immediately and 48 hours post loading. Statistical analysis was performed using one-way ANOVA with Turkey's post hoc test. Data presented as mean±s.e.m. ***p<0.001.

FIG. 39: Effects of sPLA2i-NPs on attenuating OA pain after 9N-loading. von Frey assay on sham and 9Nload-operated knee joints with 14-day treatment at 1 or 2 weeks post loading (n=8). The data of day 0 was acquired before loading. 9N-loading was performed on 3-month-old male mice. Intra-articular injections of PBS, sPLA2i, Ctrl-NPs and sPLA2i-NPs were made immediately and 48 hours post loading. Graph lines represent: Sham (top line at 2 weeks); sPLA2i-NP (second line from the top at 2 weeks); PBS (third line from the top at 2 weeks); sPLA2i (fourth line from the top at 2 weeks); Ctrl-NP (bottom line at 2 weeks). Statistical analysis was performed using one-way ANOVA with Turkey's post hoc test. Data presented as mean±s.e.m. *p<0.05 for sPLA2i-NPs vs. PBS.

FIG. 40A is a scheme for synthesis of varespladib-lipid conjugate. FIG. 40B is a graph showing NMR characterization of the synthesized varespladib-lipid conjugate.

FIGS. 41A and 41B are characterization of varespladib-NPs. FIG. 41A is a graph showing varespladib-NP size by dynamic light scattering (DLS). FIG. 41B is a graph showing the stability of the varespladib-NPs in bovine synovial fluid.

FIGS. 42A and 42B show Safranin 0 staining (FIG. 42A) and Mankin score (FIG. 42B) of mouse joints at 3 months with PBS, Control-NPs, free varespladib, or varespladib-NPs injections starting right after DMM surgery. The bars in FIG. 42B are arranged in the following order from left to right: Sham, PBS, Control-NP, free varespladib, and varespladib-NP. The bottom images in FIG. 42A are the magnified images of the black boxed areas. Scale bars, 200 um. **P<0.01, ***P<0.001, and ****P<0.0001.

### DETAILED DESCRIPTION OF ILLUSTRATIVE EMBODIMENTS

The present invention relates to compositions comprising a drug delivery system comprising a phospholipid that is hydrolyzed by phospholipase A2 (PLA2) enzyme and a drug.

Therapeutic, prophylactic, or diagnostic drugs are conjugated onto phospholipids via the sPLA2-cleavable sn-2 ester bond. To control the selective release of the drug by sPLA2, aminocaproic spacers between the drug and lipid backbone can be introduced. This synthesis method is unique and generates drug-spacer-phospholipid conjugates cleavable by PLA2. These phospholipids are then incorporated into the drug delivery systems. As but some examples, sPLA2 inhibitors, MMP-13 inhibitors or ADAMTS-5 inhibitors can be conjugated onto lipids based on this synthesis method and then incorporated into the delivery system, including liposomes or micelles.

The drug delivery systems can include phospholipids with a diagnostic drug conjugated onto the phospholipids via the sPLA2-cleavable sn-2 ester bond and incorporate therapeutic or prophylactic drugs in the drug delivery systems. The drug delivery systems can include phospholipids with a therapeutic or a prophylactic drug conjugated onto the phospholipids via the sPLA2-cleavable sn-2 ester bond and incorporate diagnostic drugs in the drug delivery systems. The drug delivery systems can include phospholipids with a therapeutic drug conjugated onto phospholipids via the sPLA2-cleavable sn-2 ester bond and another therapeutic drug in the drug delivery systems.

The drugs, including diverse drugs such as sPLA2 inhibitors, MMP-13 inhibitors or ADAMTS-5 inhibitors, can be conjugated onto lipids based on this synthesis method and then incorporated into the delivery system, including liposomes or micelles. The Examples demonstrate conjugation of two different sPLA2 inhibitors using this unique conjugation method to form phospholipids that are hydrolysable by the PLA2 enzyme. These phospholipids are incorporated into the drug delivery systems.

Also provided herein are methods for treating or for determining the location of a region to be treated or monitored in a patient in need thereof, the methods comprising: administering to the subject the disclosed composition.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, certain preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “biological” or “biological sample” refers to a sample obtained from an organism or from components (e.g., cells) of an organism. The sample can be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples can also include sections of tissues such as frozen sections taken for histological purposes.

As used herein, “higher” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control reference. A disclosed herein an expression level higher than a reference value refers to an expression level (mRNA or protein) that is higher than a normal or control level from an expression (mRNA or protein) measured in a healthy subject or defined or used in the art.

As used herein, “lower” refers to expression levels which are at least 10% lower or more, for example, 20%, 30%, 40%, or 50%, 60%, 70%, 80%, 90% lower or more, and/or 1.1 fold, 1.2 fold, 1.4 fold, 1.6 fold, 1.8 fold, 2.0 fold lower or more, and any and all whole or partial increments in between, than a control reference. A disclosed herein an expression level lower than a reference value refers to an expression level (mRNA or protein) that is lower than a normal or control level from an expression (mRNA or protein) measured in a healthy subject or defined or used in the art.

As used herein, the terms “control,” or “reference” can be used interchangeably and refer to a value that is used as a standard of comparison.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment can be in respect of an existing condition or can be prophylactic (preventative treatment). Treatment can include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that can be referred to as nucleic acids.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intra-tumoral, intravenous, intrapleural, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

A “subject” or “patient,” as used therein, can be a human or non-human mammal. Non-human mammals include, for example, livestock and pets, such as ovine, bovine, porcine, canine, feline and murine mammals. Preferably, the subject is a human. Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Provided herein are compositions and methods for treating a subject suffering from radiculopathy, neuropathy, or a disease or a condition associated with inflammation. The compositions disclosed herein comprise a drug delivery system comprising a phospholipid that is hydrolyzed by phospholipase A2 (PLA2) enzyme and a drug.

Compositions, Pharmaceutical Compositions and Formulations.

Provided herein are compositions comprising a drug delivery system comprising a phospholipid that is hydrolyzed by phospholipase A2 (PLA2) enzyme and a drug. In some embodiments, the phospholipid, when hydrolyzed, allows release of the drug from the drug delivery system.

The disclosed drug delivery system can comprise a lipid having an aliphatic group and a hydrophilic moiety. In some embodiments, the lipid is phospholipid, a lysophospholipid or a phospholipid derivative that can be a substrate of PLA2. In some embodiments, the lipid is fluorescent. In some embodiments, the phospholipid represents at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90, at least 95% by weight of the drug delivery system. In some embodiments, the phospholipid represents at least 5% or at least 10% by weight of the drug delivery system.

The term “drug delivery system” used herein encompasses nanoparticles or macromolecular structures which as the main constituent include lipid or lipid derivatives. Non-limiting examples are liposomes and micelles. In some embodiments the drug delivery system comprises a liposome or a micelle. In further embodiments the micelle or liposome includes a nanoparticle, which consists of at least one selected from the group consisting of: superparamagnetic iron oxide (SPIO), iron oxide, an iron derivative, a magnetic nanoparticle and gold nanoparticle. In yet further embodiments, the liposome or micelle includes a tracing agent comprising a radiolabel or a fluorescent dye. In other embodiments, the micelle or liposome is labeled with an antibody, peptide, protein, aptamer, or small molecule that confers specificity for a specific target.

The term “Liposome” is known as self-assembling structure comprising one or more lipid bilayers, each of which surrounds an aqueous compartment and comprises two opposing monolayers of amphipathic lipid molecules. Amphipathic lipids comprise a polar (hydrophilic) headgroup region linked to one or two non-polar (hydrophobic) aliphatic groups. Liposomes can have a single lipid bilayer (unilamellar liposomes, “ULVs”), or multiple lipid bilayers (multilamellar liposomes, “MLVs”), and can be made by a variety of methods known in the art.

Various methodologies, such as but not limited to, sonication, homogenisation, French Press application and milling can be used to prepare liposomes of a smaller size from larger liposomes.

In some embodiments, the disclosed drug delivery system (e.g. a liposome or micelle) can include other constituents which may or may not have a pharmaceutical effect, but which will render its structure more stable (or alternatively more unstable) or will protect it against clearance and will thereby increase the circulation time thereby improving the overall efficiency of a pharmaceutical drug included in it.

In some embodiments, the drug comprises at least one of a PLA2 inhibitor, an anti-inflammatory agent (e.g. steroids and non-steroids) or a neuromodulatory agent. In some embodiments, the drug comprises an antibody, a small-molecule inhibitor, a peptide inhibitor, or a siRNA. In other embodiments, the drug comprises a ribozyme, an antisense molecule, an aptamer, a peptidomimetic, a small molecule, a histone deacetylase (HDAC) inhibitor, a Janus kinase (JAK) inhibitor, an IFN inhibitor, an IFN antibody, an IFN-α Receptor 1 antibody, an IFN-α Receptor 2 antibody, a viral peptide, a checkpoint inhibitor, a PD-1 inhibitor, a PD-L1 inhibitor, a CTLA-4 inhibitor, a cytokine, a growth factor, a photosensitizing agent, a toxin, a signaling modulator, an anti-cancer antibiotic, an anti-cancer antibody, an angiogenesis inhibitor, a chemotherapeutic compound, anti-metastatic compound, an immunotherapeutic compound and a combination thereof.

In some embodiments, the drug is one or more MMP-13 inhibitors, one or more ADAMTS-5 inhibitors, and combinations thereof. Exemplary MMP-13 inhibitors are described by Vandenbroucke and Libert (Vandenbroucke and Libert, Nature Reviews Drug Discovery, 13:904-927 (2014)). Exemplary MMP-13 inhibitors include statins, including atorvastatin and mevastatin. Exemplary ADAMTS-5 inhibitors include Engineered N-TIMP-3; (2R)-N4-hydroxy-2-(3-hydroxybenzyl)-N1-[(1S,2R)-2-hydroxy-,3-dihydro-1H-inden-1-yl]butanediamides; N-hydroxyformamides; 1,2,4-triazole-3-thiol scaffolds; N-((8-Hydroxy-5-substituted-quinolin-7-yl)(phenyl)methyl)-2-phenyloxy/amino-acetamides; 5-((1H-Pyrazol-4-yl)methylene)-2-thioxothiazolidin-4-one; 4-(benzamido)-4-(1,3,4-oxadiazol-2-yl) butanoic acid; 1-sulfonylaminocyclopropanecarboxylates, N-substituted sulfonylamino-alkanecarboxylates; 1,3,5-triazine core; CRB017 (antibody against ancillary domain); and AGG-523. (Dancevic and McCulloch, Arthritis Research & Therapy, 16:429 (2014)).

The drug delivery system can act as an inert composition until reaching and interacting with the area of interest, e.g. cancerous, infected or inflammatory area or tissue. The drug delivery system can include a number of other components. In particular, the disclosed drug delivery system can further contain a component which controls the release of a drug substance, extracellular PLA2 activity controlling agents or permeability enhancer, e.g. short chain lipids and lipopolymers/glycolipids.

Examples of relevant groups of compounds that can be included in liposomes (or micelles) as modifiers are the stabilizing compound lipopolymers and glycolipids, such as lipopolymers (e.g. polyethyleneoxide-dipalmitoylphosphatidyl ethanolamine, DPPE-PEG, polyethyleneoxide-distearoylphosphatidylethanolamine, DSPE-PEG) with PEG molecular weight from 100 to 10000 Daltons. It has been shown that lipopolymers function as stabilizers for the liposome, i.e. lipopolymer increases the circulation time, and as activators for extracellular PLA2.

Liposome outer surfaces can be coated with serum proteins, such as opsonins, in mammals' circulatory systems. Without wishing to be bound by the theory, liposomes clearance can be inhibited by modifying the outer surface of liposomes such that binding of serum proteins can be inhibited. Effective surface modification (i.e. inhibition of opsonisation and retinculoendothelial systems (RES) uptake), can be accomplished by incorporating into liposomal bilayers lipids a chemical moiety which can inhibit the binding of serum proteins to liposomes such that the pharmacokinetic behavior of the liposomes in the circulatory systems of mammals is altered and the activity of extracellular PLA2 is enhanced (e.g. STEALTH® liposomes (Liposome Technology Inc., Menlo Park, Calif.) include polyethyleneglycol (PEG)-grafted lipids at about 5 mol % of the total dehydrated liposome). The polymer component of the lipid bilayer protects the liposome from uptake by the RES, and thus the circulation time of the liposomes in the bloodstream is extended.

Hydrophilic polymers suitable for use in lipopolymers are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In some embodiments, the polymers are those having a molecular weight of from about 300 daltons to about 5,000 daltons. In one preferred embodiment, the polymer is polyethyleneglycol of 750 daltons (PEG(750)). Other hydrophilic polymers which can be suitable for use herein include polyvinylpyrrolidone, polymethoxazoline, polyethyloxazoline, polyhydroxypropyl methacrylamide, polymethacrylamide, polydimethylacrylamide, and derivatised celluloses such as hydroxymethylcellulose or hydroxyethylcellulose.

The drug delivery system (e.g. liposomes) can also contain other constituents such as other lipids, sterolic compounds, polymer-ceramides as stabilizers and targeting compounds. The drug delivery system comprising lipid derivatives can include other lipids as well. Other lipids are selected for their ability to adapt compatible packing conformations with the lipid derivative components of the bilayer such that the all the lipid constituents are tightly packed, and release of the lipid derivatives from the bilayer is inhibited. Lipid-based factors contributing to compatible packing conformations are well known to ordinarily skilled artisans and include, without limitation, acyl chain length and degree of unsaturation, as well as the headgroup size and charge.

Other lipids can be suitable as derivatives, including but not limited to phosphatidylethanolamines (“PEs”) such as egg phosphatidylethanolamine (“EPE”) or ordioleoyl phosphatidylethanolamine (“DOPE”).

Lipids can also be modified by headgroup derivatization with dicarboxylic acids, such as glutaric, sebacic, succinic and tartaric acids. Accordingly, suitable headgroup-derivatized lipids include phosphatidyl-ethanolamine-dicarboxylic acids such as dipalmitoyl phosphatidylethanolamine-glutaric acid (“DPPE-GA”), palmitoyloleoyl phosphatidylethanolamine-glutaric acid (“POPE-GA”) and dioleoyl phosphatidylethanolamine-glutaric acid (“DOPE-GA”).

The disclosed drug delivery system can have various diameters ranging from 5 nm to 300 nm. In some embodiments, the diameter of the drug delivery system is at least 3 nm, at least 5 nm, at least 10 nm, at least 25, at least 50 nm, at least 75 nm, at least 100 nm, at least 110 nm, at least 120 nm, at least 130, at least 140 nm, at least 150 nm, at least 160 nm, at least 170 nm, at least 180 nm, at least 190 nm, at least 200, at least 210 nm, at least 220 nm, at least 230 nm, at least 240 nm, at least 250 nm, at least 260 nm, at least 270 nm, at least 280 nm, at least 290 nm, at least 300, at least 310 nm, at least 320 nm, at least 330 nm, at least 340 nm, at least 350 nm, at least 360 nm, at least 370 nm, at least 380 nm, at least 390 nm, at least 400, at least 450 nm, at least 500 nm, at least 550 nm, at least 600 nm, at least 700, at least 750 nm, at least 800 nm, at least 850 nm, at least 900 nm, at least 950 nm, at least 1000 nm, at least 2000 nm, at least 3000, at least 4000 nm or at least 5000 nm. In some embodiments, the diameter of the drug delivery system is at least between 50 nm to at least 300 nm and any integer in between. In some embodiments, the diameter of the drug delivery system is at least between 150 nm to at least 250 nm.

Also provided herein are pharmaceutical compositions comprising the drug delivery system disclosed herein. In some aspects, the pharmaceutical compositions comprise a pharmaceutically acceptable carrier. In some embodiments, the compositions disclosed herein are used for medicaments and for the preparation of medicaments for the treatment of diseases or conditions associated with inflammation.

Such a pharmaceutical composition can be in a form suitable for administration to a subject, or the pharmaceutical composition can further comprise one or more pharmaceutically acceptable carriers, one or more additional ingredients, or some combination of these. The various components of the pharmaceutical composition can be present in the form of a physiologically acceptable salt, such as in combination with a physiologically acceptable cation or anion, as is well known in the art.

In an embodiment provided herein, the pharmaceutical composition is useful for practicing the method of the invention and can be administered to deliver a dose of between 1 ng/kg/day and 100 mg/kg/day. In another embodiment, the pharmaceutical composition can be administered to deliver a dose of between 1 ng/kg/day and 500 mg/kg/day. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition can comprise between 0.1% and 100% (w/w) active ingredient.

Pharmaceutical compositions that are useful in the methods of the invention can be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human subject being treated, and the like.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the drug delivery system or drug within can be formulated in a natural capsid, a modified capsid, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form can be for a single daily dose or one of multiple daily doses (e.g., about 1 to 4 or more times per day). When multiple daily doses are used, the unit dosage form can be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In one aspect a pharmaceutical composition is disclosed for treating a subject suffering from an injury, radiculopathy, neuropathy, cancer, shingles, a neuropathic pain or disease or a condition associated with inflammation. The pharmaceutical composition comprises a drug delivery system comprising a phospholipid that is hydrolyzed by PLA2 enzyme, a drug and a pharmaceutical acceptable carrier. Pharmaceutically acceptable carriers, which are useful, include, but are not limited to, glycerol, water, saline, ethanol and other pharmaceutically acceptable salt solutions such as phosphates and salts of organic acids. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents.

The disclosed composition can comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition can include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which can be detrimental to the shelf life of the formulation. While BHT and disodium edetate are the particularly preferred antioxidant and chelating agent respectively for some compounds, other suitable and equivalent antioxidants and chelating agents can be substituted therefore as would be known to those skilled in the art.

Administration/Dosing

The regimen of administration can affect what constitutes an effective amount. For example, the therapeutic formulations can be administered to the patient subject either prior to or after a surgical intervention related to cancer, or shortly after the patient was diagnosed with cancer. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present invention to a patient subject, preferably a mammal, more preferably a human, can be carried out using known procedures, at dosages and for periods of time effective to treat cancer in the subject. An effective amount of the therapeutic compound necessary to achieve a therapeutic effect can vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the invention is from about 0.01 and 50 mg/kg of body weight/per day.

The compound can be administered to a subject as frequently as several times daily, or it can be administered less frequently, such as once a day, once a week, once every two weeks, once a month, or even less frequently, such as once every several months or even once a year or less. It is understood that the amount of compound dosed per day can be administered, in non-limiting examples, every day, every other day, every 2 days, every 3 days, every 4 days, or every 5 days. For example, with every other day administration, a 5 mg per day dose can be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention can be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

Routes of Administration

One skilled in the art will recognize that although more than one route can be used for administration, a particular route can provide a more immediate and more effective reaction than another route.

Routes of administration of the disclosed compositions include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein. In one embodiment, treatment with the disclosed drug delivery system comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal and any combination thereof. In some embodiment, the administration route is selected from the group consisting of intramuscular (IM), subcutaneous (SC), intravenous (IV), intrathecal, and intra-arterial (IA).

In yet another aspect, also provided herein is a kit for determining the location of a region to be treated or monitored in a patient in need thereof, comprising: a composition comprising a phospholipid that is hydrolyzed by phospholipase A2 (PLA2) enzyme, optionally a drug and a tracing agent comprising a radiolabel or a fluorescent dye; and determining a location, within the subject, of any component of the composition.

Kit

The invention includes a composition comprising a phospholipid that is hydrolyzed by phospholipase A2 (PLA2) enzyme, wherein the composition comprises a tracing agent comprising a radiolabel or a fluorescent dye that is useful for the detection of a region of interest.

In certain embodiments, a kit is provided. Commercially available kits for use in these methods are, in view of this specification, known to those of skill in the art. In general, kits will comprise a detection reagent that is suitable for detecting the location of a region to be treated or monitored in a patient in need thereof

Methods of the Invention

In one aspect, disclosed herein are methods of treating a subject in need thereof, the methods comprising administering to the subject a drug delivery system comprising a phospholipid that is hydrolyzed by phospholipase A2 (PLA2) enzyme and a drug.

In some embodiments, the phospholipid, when hydrolyzed, allows the release of the drug from the drug delivery system.

In another aspect, disclosed herein are methods for determining the location of a region to be treated or monitored in a patient in need thereof. The methods comprise administering to a subject a composition comprising a phospholipid that is hydrolyzed by phospholipase A2 (PLA2) enzyme, optionally a drug; and determining a location, within the subject, of any component of the composition.

In some embodiments, the delivery system comprises a liposome or a micelle. In some embodiments, the micelle or liposome includes a nanoparticle, which consists of at least one selected from the group consisting of: superparamagnetic iron oxide (SPIO), iron oxide, an iron derivative, a magnetic nanoparticle and gold nanoparticle. In other embodiments, the micelle or liposome includes a tracing agent comprising a radiolabel or a fluorescent dye. In further embodiments, the drug comprises at least one of a PLA2 inhibitor, an anti-inflammatory agent or a neuromodulatory agent. In yet further embodiments, the drug comprises and an antibody, a small-molecule inhibitor, a peptide inhibitor, or a siRNA. In additional embodiments, the micelle or liposome is labeled with an antibody, peptide, protein, aptamer, or small molecule that confers specificity for a specific target. Exemplary molecules for labeling the micelle or liposome include antibodies with specificity to a target tissue. For example, molecules for labeling the micelle or liposome include antibodies with specificity to osteochondral tissues, chondrocytes, neural tissues, immune cells, inflamed tissues, connective tissues and tumors. Antibodies with specificity to different tissues, cells, and organs are known in the art. For example, antibodies specific to cluster differentiation (CD) 11b can be used to specifically target the micelle or liposome to monocytes, macrophages and granulocytes; antibodies specific to CD3 can be used to specifically target the micelle or liposome to T cells; and antibodies to neuronal cell surface proteins can be used to specifically target the micelle or liposome to neurons. As an example, labeling the micelle or liposome with anti-CD44 antibody can confer the micelle or liposome with a specificity for chondrocytes, cancer cells, and lymphocytes.

In other embodiments, the subject is suffering from an injury, radiculopathy, neuropathy, cancer, shingles, a neuropathic pain, osteoarthritis or disease or a condition associated with inflammation.

In some embodiments, the neuropathic pain relates to a physical injury (e.g. trauma), diabetes, vascular or blood circulation problems, autoimmune diseases, nutritional imbalance, drug therapy (e.g. chemotherapy), viral or microbial infections.

In some embodiments, the treatment of cancer provided herein can include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers can include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganlioma, meningioma, adrenalcortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Karposi's sarcoma, moles dysplastic nevi, lipoma, angioma, dermatofibroma, keloids, and psoriasis. In other embodiments, the cancer treated by the presently disclosed methods comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an oesophagus cancer or a soft tissue cancer.

In some embodiments, the cancer to be treated is selected from the group consisting of brain cancer, breast cancer, lung cancer, colon cancer, ovarian cancer, prostate, pancreatic, leukemia, lymphoma, sarcoma and carcinoma.

Reference Value or Control

The methods provided herein include comparing a measured level of PLA2 from a subject in need of a treatment to a reference value (i.e. the control amount) PLA2 level in a healthy subject.

In one embodiment, the reference level of PLA2 can be obtained by measuring the protein or expression level of PLA2 in a healthy subject. Preferably, the healthy subject is a subject of similar age, gender and race and has never been diagnosed with any type of sever disease particularly any type of neuropathy, inflammatory disease or cancer.

In another embodiment, the reference value of PLA2 is a value for expression of PLA2 that is accepted in the art. This reference value can be baseline value calculated for a group of subjects based on the average or mean values of PLA2 by applying standard statistically methods.

In one embodiment, the level of PLA2 is determined by a method selected from the group consisting of detecting mRNA of the PLA2 gene, detecting a PLA2 protein encoded by the gene, and detecting a biological activity of the protein encoded by the gene.

In certain aspects of the present invention, the level of PLA2 is determined in a sample from a subject. The sample can include diseased cells, tumor cells, any fluid from the surrounding of tumor cells (e.g. leukemic blood, or tumor tissue) or any fluid that is in physiological contact or proximity with the tumor, or any other body fluid in addition to those recited herein should also be considered to be included herein.

Combination Therapies

The compositions and methods for treating a patient in need thereof described herein can be useful when combined with at least one additional compound useful for treating a subject suffering from an injury, neuropathy, cancer, shingles, a neuropathic pain, osteoarthritis or disease or a condition associated with inflammation. The additional compound can comprise a commercially available compound, known to treat, prevent, or reduce the symptoms of the disease or condition to be treated.

In one aspect, the pharmaceutical composition disclosed herein can be used in combination with a therapeutic agent such antibiotics, antifungals, and anti-inflammatory agents such as steroids and non-steroids (e.g. aspirin, ibuprofen or naproxen).

In another aspect, the pharmaceutical composition disclosed herein can be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein can be used in combination with a radiation therapy.

Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine.

Other examples of chemotherapeutic agents include, but are not limited to, the following and their pharmaceutically acceptable salts, acids and derivatives: MEK inhibitors, such as but not limited to, refametinib, selumetinib, trametinib or cobimetinib; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK); razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and docetaxel (TAXOTERE, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine.

An anti-cell proliferation agent can further be defined as an apoptosis-inducing agent or a cytotoxic agent. The apoptosis-inducing agent can be a granzyme, a Bch 2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

An immunotherapeutic agent can be, but is not limited to, an interleukin-2 or other cytokine, an inhibitor of programmed cell death protein 1 (PD-1) signaling such as a monoclonal antibody that binds to PD-1, Ipilimumab. The immunotherapeutic agent can also block cytotoxic T lymphocytes associated antigen A-4 (CTLA-4) signaling and it can also relate to cancer vaccines and dendritic cell-based therapies.

In one embodiment the subject suffering from cancer is administered at least one anti-cancer therapeutic agent selected from the group consisting of: a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an engineered anti-cancer virus or virus derivative and a combination of any thereof. In one embodiment, the least one anti-cancer therapeutic agent is administered formerly, simultaneously or subsequently to the administering of the presently disclosed drug delivery system.

In one embodiment, the subject is administered an inhibiting agent known in the art for inhibiting, suppressing or reducing partially or fully and temporarily or permanently a given immune pathway. The inhibiting agent comprises siRNA, ribozyme, an antisense molecule, an aptamer, a peptidomimetic, a small molecule, histone deacetylase (HDAC) inhibitor, Janus kinase (JAK) inhibitor, IFN inhibitor, IFN antibody, IFN-α Receptor 1 antibody, IFN-α Receptor 2 antibody and viral peptide and a combination of any thereof. The viral peptide can be, but not limited to, NS1 protein from an Influenza virus or NS2B3 protease complex from dengue virus.

### Aspects

The disclosure may be described with the following exemplary Aspects:

Aspect 1. A composition, comprising: a drug and a drug delivery system comprising a phospholipid that is hydrolyzed by phospholipase A2 (PLA2) enzyme.

Aspect 2. The composition of Aspect 1, wherein the phospholipid, when hydrolyzed, allows release of the drug from the drug delivery system.

Aspect 3. The composition of any one of Aspects 1-2, wherein the phospholipid represents at least 10% by weight of the drug delivery system.

Aspect 4. The composition of any one of Aspects 1-3, wherein the drug delivery system comprises a liposome or a micelle.

Aspect 5. The composition of any one of Aspects 1-4, wherein the micelle or liposome includes a nanoparticle, which consists of at least one selected from the group consisting of: superparamagnetic iron oxide (SPIO), iron oxide, an iron derivative, a magnetic nanoparticle and gold nanoparticle.

Aspect 6. The composition of any one of Aspects 1-5, wherein the liposome or micelle includes a tracing agent comprising a radiolabel or a fluorescent dye.

Aspect 7. The composition of any one of Aspects 1-6, wherein the drug comprises at least one of a PLA2 inhibitor, an anti-inflammatory agent or a neuromodulatory agent.

Aspect 8. The composition of any one of Aspects 1-7, wherein the drug comprises an antibody, a small-molecule inhibitor, a peptide inhibitor, or an siRNA.

Aspect 9. The composition of any one of Aspects 1-8, wherein the micelle or liposome is labeled with an antibody, peptide, protein, aptamer, or small molecule that confers specificity for a specific target.

Aspect 10. A pharmaceutical composition, comprising the drug delivery system of any one of claims 1-9 and a pharmaceutically acceptable carrier.

Aspect 11. The composition of any one of Aspects 1-10, for use as a medicament.

Aspect 12. The composition of Aspect 11, for the preparation of a medicament for the treatment of diseases or conditions associated with inflammation.

Aspect 13. A method for treating a subject in need thereof, the method comprising: administering to the subject a drug delivery system comprising (i) a phospholipid that is hydrolyzed by phospholipase A2 (PLA2) enzyme and (ii) a drug.

Aspect 14. The method of Aspect 13, wherein the phospholipid, when hydrolyzed, allows the release of the drug from the drug delivery system.

Aspect 15. The method of any one of Aspects 13-14, wherein the phospholipid represents at least 5% by weight of the drug delivery system.

Aspect 16. The method of any one of Aspects 13-15, wherein the drug delivery system comprises a liposome or a micelle.

Aspect 17. The method of any one of Aspects 13-16, wherein the micelle or liposome includes a nanoparticle, which consists of at least one selected from the group consisting of: superparamagnetic iron oxide (SPIO), iron oxide, an iron derivative, a magnetic nanoparticle and gold nanoparticle.

Aspect 18. The method of any one of Aspects 13-17, wherein the micelle or liposome includes a tracing agent comprising a radiolabel or a fluorescent dye.

Aspect 19. The method of any one of Aspects 13-18, wherein the drug comprises at least one of a PLA2 inhibitor, an anti-inflammatory agent or a neuromodulatory agent.

Aspect 20. The method of any one of Aspects 13-19, wherein the drug comprises an antibody, a small-molecule inhibitor, a peptide inhibitor, or an siRNA.

Aspect 21. The method of any one of Aspects 13-20, wherein the micelle or liposome is labeled with an antibody, peptide, protein, aptamer, or small molecule that confers specificity for a specific target.

Aspect 22. The method of any one of Aspects 13-21, wherein the subject is suffering from an injury, radiculopathy, neuropathy, cancer, shingles, a neuropathic pain, osteoarthritis or disease or a condition associated with inflammation.

Aspect 23. The method of Aspect 22, wherein the cancer is selected from the group consisting of brain cancer, breast cancer, lung cancer, colon cancer, ovarian cancer, prostate, pancreatic, leukemia, lymphoma, sarcoma and carcinoma.

Aspect 24. The method of any one of Aspects 13-23, wherein the administration route is selected from the group consisting of intramuscular (IM), subcutaneous (SC), intravenous (IV), intrathecal, and intra-arterial (IA).

Aspect 25. The method of Aspect 24, wherein a location of the local administration comprises a higher level of PLA2 as compared to a control PLA2 level in a healthy subject.

Aspect 26. The method of any one of Aspects 13-25, further comprising determining a location, within the subject, of the drug delivery system.

Aspect 27. The method of Aspect 26, wherein the determining comprises magnetic resonance imaging, fluorescent imaging, computed tomography, nuclear imaging, or ultrasound.

Aspect 28. A method for determining the location of a region to be treated or monitored in a subject in need thereof, the method comprising: administering to the subject a composition comprising a phospholipid that is hydrolyzed by phospholipase A2 (PLA2) enzyme, optionally comprising a drug; and determining a location, within the subject, of any component of the composition.

Aspect 29. The method of Aspect 28, the composition comprises a liposome or a micelle.

Aspect 30. The method of any one of Aspects 28-29, wherein the micelle or liposome includes a nanoparticle, which consists of at least one selected from the group consisting of: superparamagnetic iron oxide (SPIO), iron oxide, an iron derivative, a magnetic nanoparticle and gold nanoparticle.

Aspect 31. The method of any one of Aspects 28-30, wherein the liposome or micelle includes a tracing agent comprising a radiolabel or a fluorescent dye.

Aspect 32. The method of any one of Aspects 28-31, wherein the determining comprises magnetic resonance imaging, fluorescent imaging, computed tomography, nuclear imaging, or ultrasound.

Aspect 33. The method of any one of Aspects 28-32, wherein the composition becomes traceable upon hydrolyzation of the phospholipid by PLA2.

Aspect 34. The method of any one of Aspects 28-33, wherein the subject is suffering from an injury, radiculopathy, neuropathy, cancer, shingles, a neuropathic pain, osteoarthritis or disease or a condition associated with inflammation.

Aspect 35. The method of any one of Aspects 13-34, wherein the subject is a human.

Aspect 36. The method of any one of Aspects 13-35, wherein the subject has osteoarthritis (OA).

Aspect 37. The composition of any one of Aspects 1-12, or the method of any one of Aspects 13-36, wherein the drug is a PLA2 inhibitor.

Aspect 38. The composition of any one of Aspects 1-12 or 37, or the method of any one of Aspects 13-36, wherein the drug is a PLA2 inhibitor selected from the group consisting of thioetheramide-PC and varespladib.

Aspect 39. The composition of any one of Aspects 1-12, 37, or 38, or the method of any one of Aspects 13-36, wherein the drug is an inhibitor of matrix metalloproteinase (MMP) and/or a Disintegrin-like And Metalloproteinase domain with Thrombospondin-1 repeats (ADAMTS) enzymes.

Aspect 40. The composition of any one of Aspects 1-12 or 37-39, or the method of any one of Aspects 13-36, wherein the drug delivery system comprises a liposome or a micelle labeled with an antibody molecule specific for a target tissue.

Aspect 41. The composition of any one of Aspects 1-12 or 37-40, or the method of any one of Aspects 13-36, wherein the drug delivery system does not include a PLA2 inhibitor.

Aspect 42. The composition of any one of Aspects 1-12 or 37-41, or the method of any one of Aspects 13-36, wherein the drug in the drug delivery system is conjugated to the phospholipid.

Aspect 43. The composition of any one of Aspects 1-12 or 37-42, or the method of any one of Aspects 13-36, wherein the drug in the drug delivery system is conjugated to the phospholipid via a spacer or a linker. A spacer or linker can comprise, e.g., aminocaproic residues, but this is not a requirement. A linker can comprise an amino acid, as well as an analogue or derivative thereof.

Aspect 44. The composition of any one of Aspects 1-12 or 37-43, or the method of any one of Aspects 13-36, wherein the drug in the drug delivery system is conjugated to the phospholipid via a spacer or a linker comprising one or more aminocaproic residues, e.g., two, three, four, five, six, seven, eight, nine, or more such residues.

Aspect 45. The composition of any one of Aspects 1-12 or 37-44, or the method of any one of Aspects 13-36, wherein the drug in the drug delivery system is conjugated to the phospholipid via a spacer or a linker that comprises an aminocaproic residue.

Aspect 46. The composition of any one of Aspects 1-12 or 37-45, or the method of any one of Aspects 13-36, wherein the drug in the drug delivery system is conjugated to the phospholipid via a spacer or a linker that forms an sn-2 ester bond with the phospholipid.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods for Examples 1-6

Materials

1-Palmitylthio-2-palmitoylamido-1,2-dideoxy-sn-glycero-3-phosphorylcholine (thioetheramide-PC, TEA-PC) was purchased from Cayman Chemical (Ann Arbor, Mich.). Hydrogenated soy phosphatidylcholine (HSPC), 1,2-distearoyl-sn-glycero-3-phosphoethanolamine-N-[methoxy(polyethylene glycol)-2000] (DSPE-PEG2000), and 1-palmitoyl-2-{6-[(7-nitro-2-1,3-benzoxadiazol-4-yl)amino]hexanoyl}-sn-glycero-3-phosphocholine (NBD-PC) were purchased from Avanti Polar Lipids, Inc. Phospholipase A₂ (PLA₂) enzyme from Naja mossambica was purchased from Sigma-Aldrich Co. 3′3 diaminobenzidine (DAB) was purchased from Vector Inc. Goat polyclonal antibody to sPLA₂-IIA and rabbit polyclonal antibody to Iba1 was purchased from Santa Cruz Biotechnology Inc and Wako, respectively. All other chemicals were used as received. All of the buffer solutions were prepared with deionized water.

### Synthesis of Hydrophobic 7 nm Superparamagnetic Iron Oxide Nanoparticles SPIOs

Oleic acid coated SPIOs were prepared by thermal decomposition as previously described.⁴⁷ After allowing the reaction to cool to room temperature, acetone was added and the sample was centrifuged to precipitate the nanoparticles. The particles were further washed in hexane and precipitated again using acetone followed by centrifugation. This washing procedure was repeated until the supernatant was clear. The particles were then allowed to air dry and dissolved in toluene at about 40 mg/mL.

### Synthesis of PLA2 Inhibitor and SPIO-Loaded Micelles

sPLA2inhibitor-loaded micelles were prepared using an oil-in-water emulsion method.⁴⁷ A mixture (230 μL) containing HSPC (1 mg in 20 μL chloroform), PLA2 inhibitor thioetheramide-PC (1 mg in 100 μL ethanol), DSPE-PEG2000 (0.25 mg in 10 μL chloroform) and SPIO NPs (2 mg in 100 μL toluene) was injected into a glass vial containing 4 mL of water, and the sample was sonicated (Branson Ultrasonics, Danbury, Conn., USA) until a homogenous mixture was achieved. The toluene and chloroform were then allowed to evaporate overnight. sPLA₂ inhibitor and SPIO-loaded phospholipid micelles samples were then centrifuged (Eppendorf Microcentriguge 5418) at 1000 rpm for 30 minutes to remove large aggregates. In order to remove empty micelles, the resulting supernatant was then centrifuged at 10,000 rpm for half hour, and the pellet was resuspended in water. The stock solution of sPLA₂ inhibitor and SPIO-loaded phospholipids micelles was stored in the dark at 4° C. The unloaded micelles, i.e. without PLA₂ inhibitor, were prepared using the similar procedures as sPLA₂ inhibitor and SPIO-loaded phospholipids micelles.

In Vitro PLA₂ Response Study

To study in vitro PLA₂ response of sPLA₂ inhibitor-loaded micelles, a 80 mol % HSPC/20 mol % NBD-PC mixture was prepared in chloroform in a glass vial. The total amount of HSPC was 1 mg. The solvent was removed using a direct stream of nitrogen prior to vacuum desiccation for a minimum of 4 h. 0.2 mL water was then added to the dried lipid film and incubated in a 50° C. water bath for 0.5 h and then sonicated for another 0.5 h at the same temperature. The stock solution of NBD-incorporated liposomes was stored in the dark at 4° C.

Measurements of the PLA₂ enzyme induced dequenching of NBD trapped within the liposomes were carried out as follows: 20 μL of NBD-incorporated liposomal suspension ([HSPC]=1 mg/mL) was added to 0.48 mL of 0.01M HEPES (pH 7.4) buffer solution containing 2 mM CaCl₂. After 5 minutes, the preincubation mixture (10 μL sPLA₂ inhibitor-loaded micelles [TEA-PC=0.25 mg/mL]+6.67 μL PLA₂ enzyme [PLA₂=7.5 U/mL], 20 minutes incubation) was added to NBD-incorporated liposomal suspension. The fluorescence intensity at 520 nm was measured using an excitation at 460 nm. The amount of NBD dequenched (% NBD dequenched) was calculated as ([Ix−I₀]/[Iₜ−I₀])×100, where I₀ is the fluorescence intensity of the liposomal suspension containing NBD at the initial time, Ix is the fluorescence intensity at any given time, and Iₜ is the fluorescence intensity after addition of 20 μL Triton X-100 (50 mM) to the suspension at the end of experiment. A similar approach was used to study the PLA₂ response of control nanoparticles (i.e., without sPLA₂-inhibitor).

In Vitro Cytotoxicity

To assess in vitro cytotoxicity to neural cells, dorsal root ganglia (DRG) were isolated from embryonic day 18 Sprague-Dawley rats and stored in Hibernate-E medium (Thermo-Scientific, Waltham, Mass.) supplemented with 2% B-27 and 0.5 mM GlutaMAX (Thermo-Scientific, Waltham, Mass.) for two days until dissociation. To perform DRG dissociation, Hibernate-E Medium was aspirated and DRGs were incubated with warm 0.25% trypsin in EDTA for 1 hour at 37° C. After that, feeding medium (Neurobasal supplemented with 2% B-27, 0.5 mM GlutaMAX, 1% fetal bovine serum (FBS), 0.02 μg/mL nerve growth factor (NGF), 2.5 mg/mL glucose, and 40 μM M2 (5-fluoro-2′-deoxyridine 10 mM, uridine 10 mM) with an additional 5% FBS was added to inhibit trypsin activity. DRGs were then manually titurated with a pestle, centrifuged at 1000 rpm for 5 min at room temperature, isolated from their supernatant, and then reconstituted with fresh feeding medium. Dissociated DRGs were then plated (3.97×10⁶ cells/mL) onto a 24-well plastic tissue culture plate treated with poly-D-lysine (PDL) and laminin. Feeding medium was changed at day-in-vitro (DIV) 1 and DIV 3. On DIV 4, cultures were separately incubated for 24 hours with micelles at different PLA₂ inhibitor concentrations: 0, 1, 5, 10, 20, 50, 100, 200, 250 μg/mL. Cell viability was tested in triplicate for each concentration using a CellTiter 96 AQ One Solution Cell Proliferation Assay (Promega, Madison, Wis.). Cytotoxicity measurements from treated groups were normalized to untreated controls as previously described.^{63, 64} Average percent cell viability was compared using a one-way ANOVA (group) with Tukey's test.

In Vivo Injury and sPLA₂ Inhibitor-Loaded Micelles Administration

Spinal sPLA₂ expression was characterized after a painful root injury. As such, rats underwent either a nerve root compression that was painful (15-minute; n=11) or a compression that served as a control since it was nonpainful (3-minute; n=11); sham injuries (n=10) were also included as surgical controls. Under isoflurane anesthesia (4% induction, 2% maintenance), male Holtzman rats underwent a painful nerve root compression in which the right C7 nerve root was surgically exposed and compressed for 15 minutes with a 10 g force microvascular clip as previously described.⁶⁵

To test the effectiveness of sPLA₂ inhibitor-loaded micelles in preventing the pain that develops from a neuropathic injury, sPLA₂ inhibitor-loaded micelles were administered in vivo after a painful nerve root compression.⁶⁵ Immediately after tissue compression, two separate groups of rats received either a 100 μl (TEA-PC concentration: 0.25 mg/mL) of sPLA₂ inhibitor-loaded micelles in saline (PLA₂-inhibitor loaded micelles; n=5), or a comparable dose of control micelles without any PLA₂ inhibitor loaded in saline (unloaded micelles; n=3) administered directly to the nerve root. After each surgery, incisions were closed using 3-0 polyester suture and surgical staples and the rats were monitored during recovery in room air.

In a second study, separate groups of rats received either 100 μL of sPLA₂ inhibitor-loaded micelles (TEA-PC concentration: 0.25 mg/mL) (sPLA₂ inhibitor-loaded micelles; n=5) or a comparable dose of unloaded control micelles (unloaded micelles; n=4) diluted in 400 μL of PBS administered intravenously through the tail vein artery after the nerve root compression on both day 1 and day 2.¹⁷ An additional group of rats was included as sham-surgical controls (sham+PLA₂-inhibitor loaded micelles; n=4) in which rats received identical surgical procedures in which the nerve root was exposed but not compressed.

Behavior Testing for Pain Sensitivity

Behavioral sensitivity was assessed by measuring mechanical hyperalgesia in the ipsilateral and contralateral forepaws, as previously detailed.⁶⁵ Testing was performed at baseline before injury (day 0) and daily for 7 days after, and was quantified as the paw withdrawal threshold to an applied mechanical stimulus. The withdrawal threshold was taken as the lowest von Frey filament to elicit a response and was confirmed by the next filament also provoking a response.⁶⁶ Testing sessions consisted of 3 rounds each separated by 10 minutes of rest. The withdrawal thresholds were separately averaged across rounds for each day, and differences in thresholds between groups were compared separately for the ipsilateral and contralateral forepaw using a repeated-measures ANOVA with post-hoc Tukeys test.

Tissue Harvest, Prussian Blue Staining and Immunohistochemistry

For the studies characterizing sPLA₂ expression in the bilateral spinal cord, tissue at the C7 level was separately harvested on days 1 (painful n=6; nonpainful n=5; sham n=5) and day 7 (painful n=5; nonpainful n=6; sham n=5). For all micelle treatment groups, on post-operative day 7 after behavior testing, the C7 nerve roots, DRG and spinal cord were harvested for histology and immunolabeling. Rats were deeply anesthetized with sodium pentobarbital (65 mg/kg) and transcardially perfused with phosphate buffered saline (PBS) followed by 4% paraformaldehyde. Subsequently, all neural tissues were post-fixed in 4% paraformaldehyde overnight, cryoprotected in 30% sucrose and embedded in OCT medium (VWR, Bridgeport, N.J.). All nerve root, DRG and spinal cord tissues were cryosectioned along the long-axis at 14 μm and thaw-mounted on to slides.

To assess micelle localization in the roots, Prussian Blue staining, was used to detect SPIOs via iron oxide staining,¹⁷ nerve root sections were incubated with 0.3% hydrogen peroxide in PBS and a 1:1 mixture of 2% potassium ferrocyanide and 2% hydrochloric acid for 30 minutes. Visualization of iron in the tissue was enhanced by 3′3 diaminobenzidine (DAB) (Vector, Burlingame, Calif.) for an additional 15 minutes. Tissue sections were then counterstained with eosin (Sigma Aldrich, St. Louis, Mo.), cleared with Xylene (Sigma Aldrich) and cover-slipped using DPX Mounting media (Sigma Aldrich).

To determine the expression of sPLA₂ and the extent of Iba1 positive microglia in the spinal cord and sPLA₂ expression in the DRG after micelle treatment, sections were blocked using 10% normal donkey serum (Vector, Burlingame, Calif.) with 0.3% Triton-X 100 and incubated in goat anti-sPLA₂ IIA (1:500; Santa Cruz, Dallas Tex.) and rabbit anti-Iba1 (1:1000, Wako, Osaka Japan) primary antibodies. Tissue sections were then rinsed with PBS and incubated for 2 hours at room temperature in donkey anti-goat 488 and donkey anti-rabbit 546 (1:500, Invitrogen, Carlsbad Calif.) secondary antibodies.

For each rat, 3-6 sections were captured using a Leica DM6000 (Wetzlar, Germany) with deconvolution at 20× and images were cropped to (750×200 pixels²) to contain the superficial (I and II) laminae of the dorsal horn. Images of the DRG were cropped (450×450 pixels) to include 10-20 random neurons per image for intensity analysis. A custom densitometry MATLAB script (Mathworks; Natick, Mass.) was used to quantify the extent of positive spinal sPLA₂ and Iba1 labeling and sPLA₂ labeling in the DRG, as previously described.^{67, 68} DRG and spinal cord sections from naïve rats (n=2) were acquired and used to set the pixel intensity thresholds used to quantify positive labeling for sPLA₂ and Iba1 separately. The number of pixels positive for either sPLA₂ or Iba1 was calculated as a percent of the total dorsal horn area for each label and similarly, the positive pixels for sPLA₂ were evaluated over the area of selected DRG neurons. Total levels of each of sPLA₂ or Iba1 labeling were then separately compared to the normal levels and expressed as a fold-change over normal levels for each rat and anatomic location. Differences in normalized intensity between the sPLA₂ inhibitor-loaded micelles and the unloaded micelles were compared using a separate Student t-tests for each label in each of the DRG and spinal cord.

Instrumentation

Fluorescence spectra measurements were made on a SPEX FluoroMax-3 spectrofluorometer (Horiba Jobin Yvon). Dynamic light scattering (DLS) measurements were performed on a Zetasizer Nano (Malvern Instruments). The scattering angle was held constant at 90°. Transmission electron microscopy (TEM, JEM-1010) was used to characterize the size and shape of the small hydrophobic SPIO and SPIO-loaded phospholipid micelles. T2 relaxation times were determined using a Bruker mq60 MR relaxometer operating at 1.41 T (60 MHz). The Fe concentration in samples was determined by ICP-OES analysis using a Genesis ICPOES (Spectro Analytical Instruments GMBH; Kleve, Germany).

### Example 1: Advantage of the PLA2-Responsive Drug Delivery System

Recently, nanoparticles for targeted drug delivery has been extensively explored in the treatment of various diseases including cancer. Such drug delivery systems not only increase the concentration of drug that can be delivered to target tissues, lowering the overall dose needed, but also reduce off-target toxicity and side-effects.

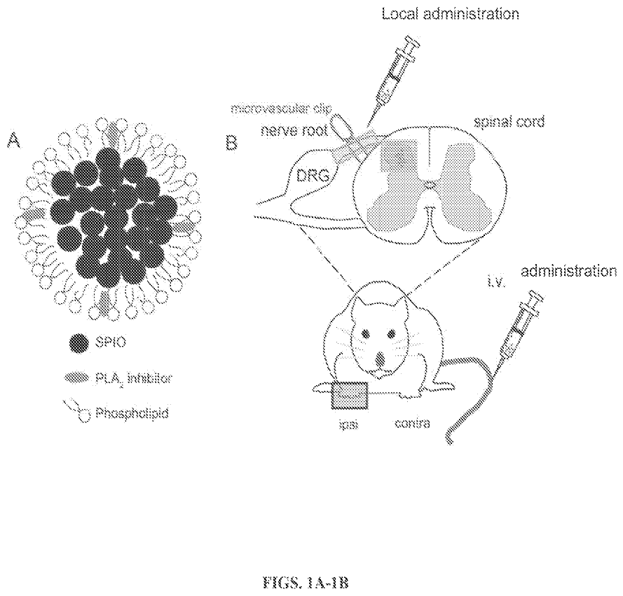

A delivery platform that enables tunable drug release based on the pathological conditions that are induced by the pain state itself, offers a unique and novel long-term clinical option to treat patients with neuropathic pain. Therefore, this study first defined whether a spinal sPLA₂ expression after a nerve root compression injury can be sensitive to a painful condition, enabling its possible utility as a potential therapeutic target for pain modulation. Based on the positive findings, sPLA₂ inhibitor-loaded micelle nanoparticles were fabricated (FIG. 1A). The effectiveness of these sPLA₂ inhibitor-loaded micelles in both preventing and abolishing pain and spinal inflammation using both local and systemic administration paradigms was tested in a well-established rodent model of persistent neuropathic pain (FIG. 1B).

### Example 2: Spinal sPLA₂ Expression after Painful Neuropathic Injury to the Nerve Root

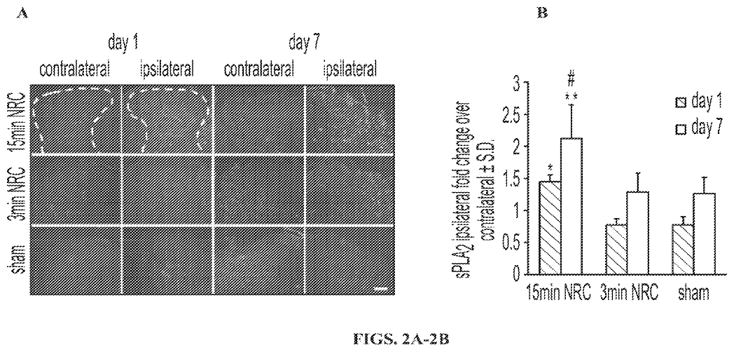

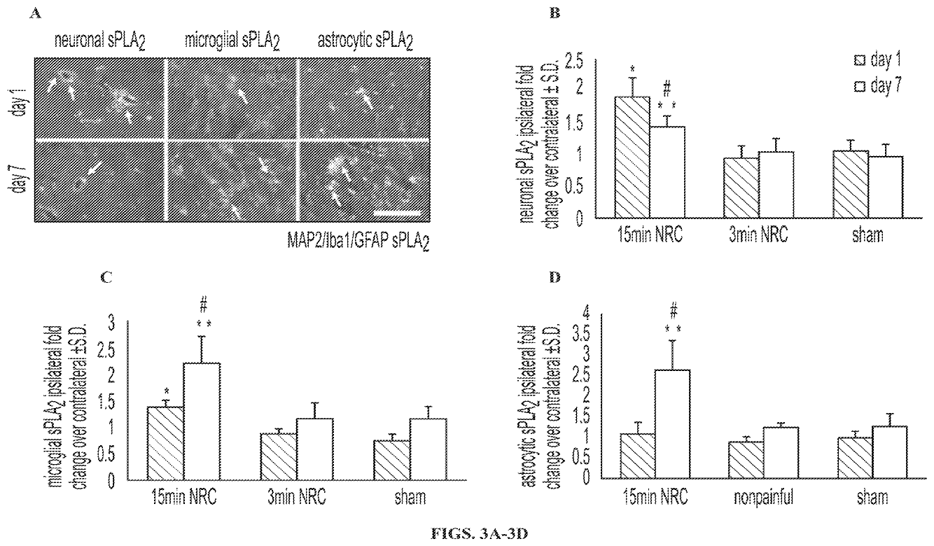

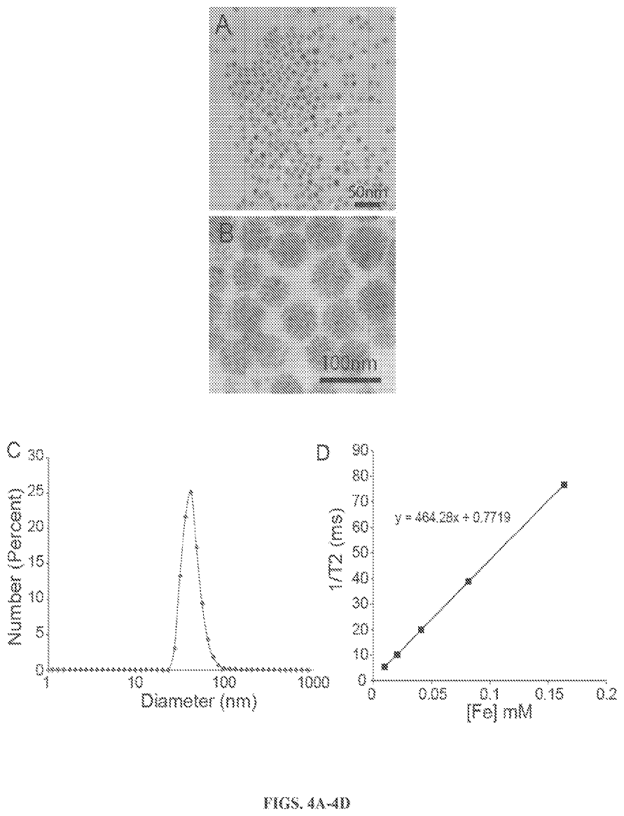

Spinal sPLA₂ is elevated only after a painful nerve root injury. In fact, early (at day 1) after nerve root compression (FIG. 2A), spinal sPLA₂ increases in the spinal cord only after a painful compression, with the extent of spinal sPLA₂ 1.85±1.20-fold greater than levels at that time after either nonpainful root compression (p=0.048) and 1.97±0.82 times the levels after a nonpainful sham surgery (p=0.044) (FIG. 2B). Further, at day 7, the increase in spinal sPLA₂ expression is even more robust with further increases (p=0.043) by 48.7±9.4% over levels at day 1. Although sPLA₂ levels in both the nonpainful and sham control groups increase at day 7 from levels at day 1, those increases are not significant (FIG. 2B). Together, these data support that increased spinal sPLA₂ expression appears to be sensitive to a painful injury and can be an appropriate molecular target for neuropathic pain treatment.

### Example 3: Synthesis and Characterization of sPLA₂ Inhibitor-Loaded Phospholipid Micelles

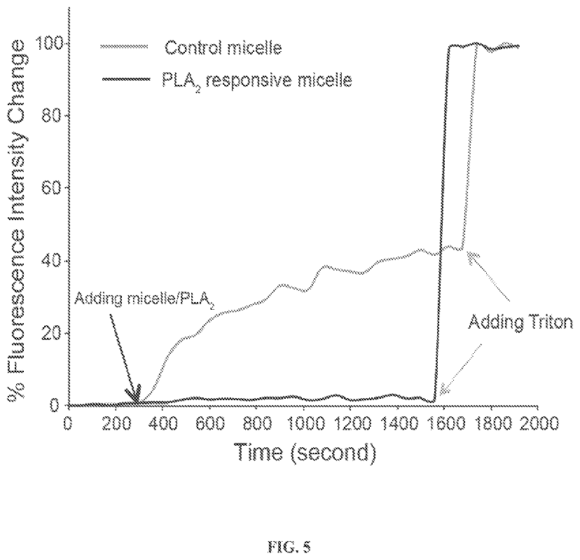

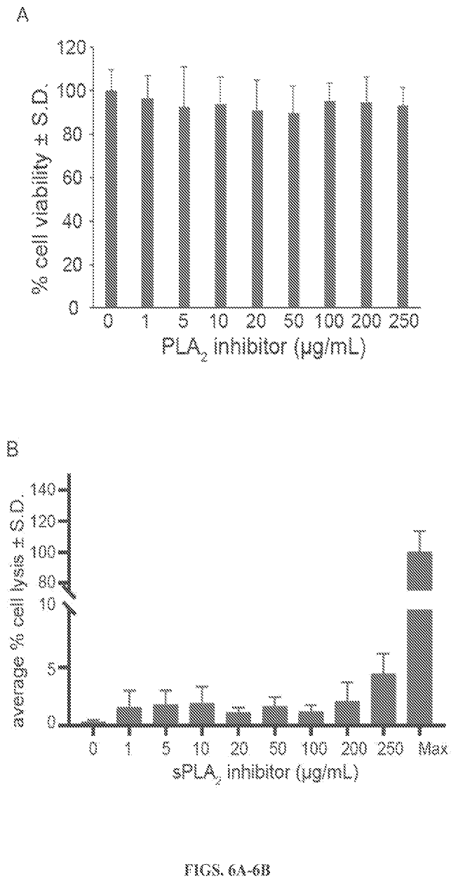

Based on the findings that spinal sPLA2 expression increases after painful neuropathic injury to the nerve root, therapeutic nanoparticles were prepared by incorporating sPLA₂ inhibitor thioetheramide-PC (TEA-PC) into small hydrophobic superparamagnetic iron oxide nanoparticle (SPION, 7 nm, FIG. 4A)-loaded phospholipid micelles. Due to the amphiphilic nature of phospholipid molecules, these SPIO-loaded micelles were highly soluble in aqueous solutions. Dynamic light scattering (DLS) revealed that the sPLA₂ inhibitor-loaded phospholipid micelles had a mean diameter of 60 nm with a polydispersity index (PDI) of 0.20 in water (FIG. 4C). The morphologies of these micelles were further confirmed by transmission electron microscopy (TEM) (FIG. 4B) in which the SPIONs were found to be tightly packed into a spherical core. The SPIO-loaded micelles exhibited superparamagnetic properties with an r2 value of 464 mM⁻¹s⁻¹ (FIG. 4D).

To study the ability of sPLA₂ inhibitor-loaded micelles to inhibit sPLA₂ enzyme, phospholipid hydrogenated soy phosphatidylcholine (HSPC) liposomes doped with the fluorescent lipid 1-palmitoyl-2-{6-[(7-nitro-2-1,3-benzoxadiazol-4-yl)amino]hexanoyl}-sn-glycero-3-phosphocholine (NBD-PC) were prepared as a fluorescent sensor for sPLA₂ activity. The fluorophore NBD was attached to the sn-2 position of the phospholipid with a spacer (C6) between NBD and the lipid backbone since native PLA₂ enzymes specifically recognize and catalytically hydrolyze the sn-2 acyl bond of phospholipids⁴⁷. When 20 mol % NBD-PC was incorporated into HSPC liposomal membrane, the NBD was highly quenched within the phospholipid membrane.⁴⁷ Little to no change in fluorescence was observed over a 24 h time period when these NBD-incorporated liposomes were incubated in buffer (10 mM HEPES, pH 7.4). However, a significant increase in the fluorescence intensity was observed immediately upon the addition of the preincubation mixture of control unloaded micelles (i.e., no PLA₂ inhibitor) and sPLA₂ enzyme (FIG. 5). The PLA₂-induced change in fluorescence was due to the release of NBD into the surrounding bulk solution upon the hydrolysis of the phospholipid liposomal membrane by PLA₂. The released NBD led to fluorescence dequenching, resulting in a higher fluorescence intensity. For comparison, sPLA₂ inhibitor-loaded micelles were also tested under similar conditions. No change in fluorescence intensity was observed when the preincubation mixture of sPLA₂ inhibitor-loaded micelles and sPLA₂ enzyme was added into NBD-incorporated liposomal suspension, revealing that the release of NBD was significantly inhibited.

To assess cytotoxicity of the sPLA₂ inhibitor-loaded micelles prior to in vivo administration, micelles were incubated for 24 hours with dorsal root ganglia neurons. Incubation of the sPLA₂ inhibitor-loaded micelles did not reduce neuronal viability at any concentration tested compared to untreated control cultures (FIG. 6A). Additionally, incubation of the sPLA₂ inhibitor-loaded micelles in neuronal cultures did not significantly increase cell lysis compared to control cultures, with the greatest concentration of micelles inducing 4.42±1.77% lysis for the total culture (FIG. 6B). These data suggest that the sPLA₂ inhibitor-loaded micelles s are safe for in vivo administration.

### Example 4: Therapeutic Efficacy Following Local Administration of sPLA₂ Inhibitor-Loaded Micelles

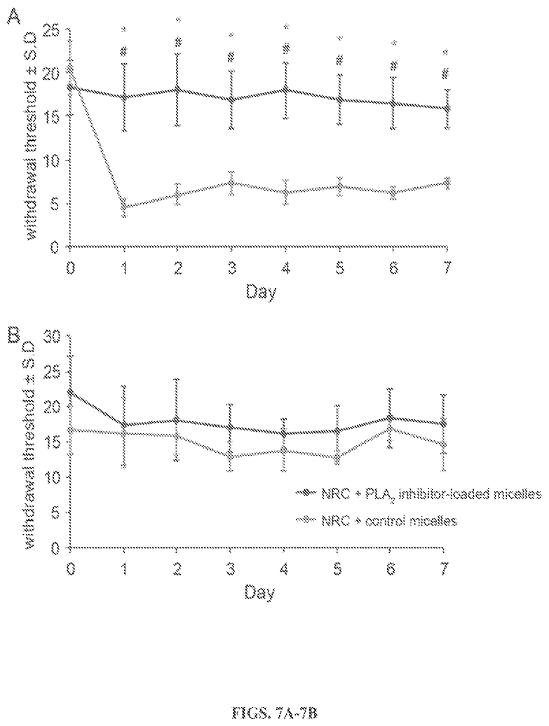

Local administration of sPLA₂ inhibitor-loaded micelles immediately after a painful root compression prevents the onset of pain as early as 1 day after injury, lasting for up to 7 days (FIG. 7A). Withdrawal thresholds in the ipsilateral forepaw after treatment with sPLA₂ inhibitor-loaded micelles are significantly higher (p<0.016) than those thresholds after injury treated with unloaded micelles on all days of testing. Treatment with the control unloaded micelles does not prevent pain development, with ipsilateral paw withdrawal thresholds on all days (days 1-7) remaining significantly lower (p<0.0001) than the pre-injury response. Additionally, there are no significant differences between thresholds in the contralateral paws for both group for any post-treatment day (FIG. 7B).

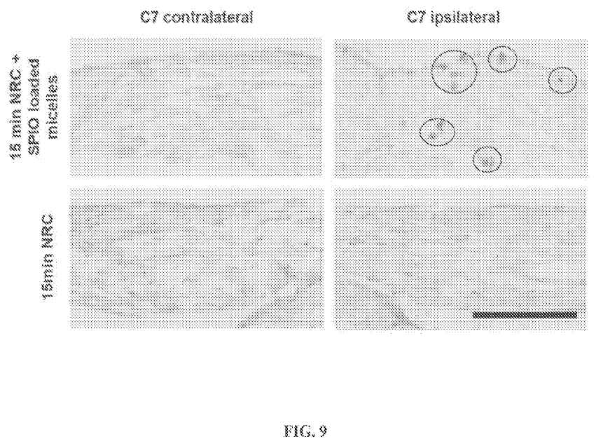

Prussian Blue staining was performed to identify if iron oxide particles are present in the injured C7 dorsal nerve roots. Following local administration of the sPLA₂ inhibitor-loaded micelles, iron oxide particles are evident only in the injured (compressed) nerve root (FIG. 9), confirming accumulation of micellar treatment at the injury site. There are no iron oxide particles evident in any contralateral uninjured nerve root after either micelle treatment.

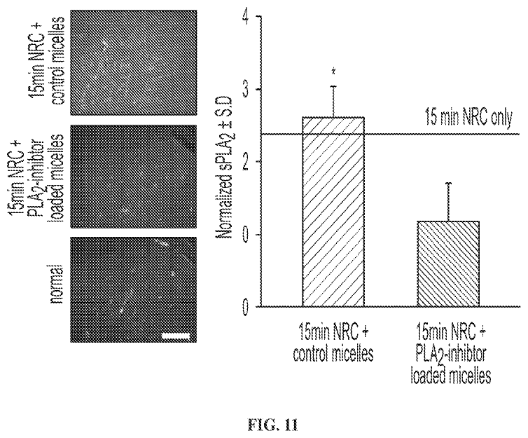

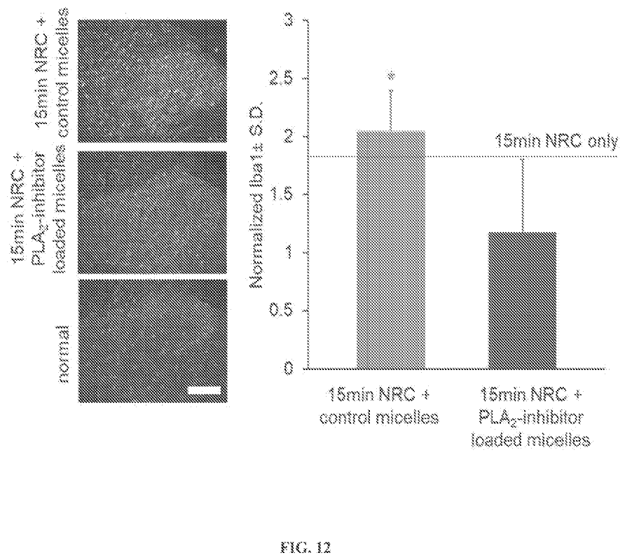

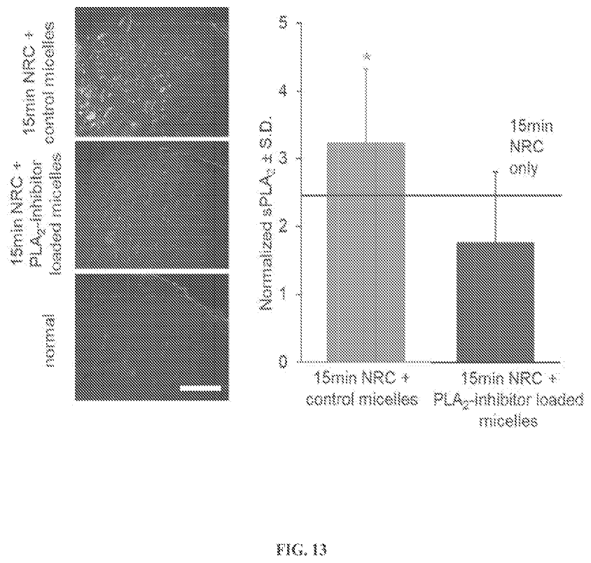

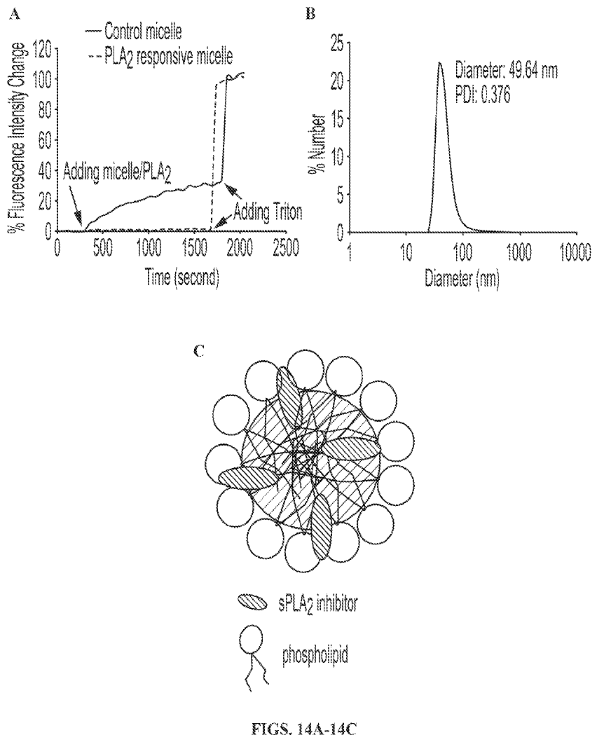

Local administration of sPLA₂ inhibitor-loaded micelles immediately after injury not only prevents pain but also prevents the increase in spinal sPLA₂ that is observed after a painful nerve root injury (FIG. 11). Spinal sPLA₂ expression at day 7 is significantly lower (*p<0.0001) after treatment compared to sPLA₂ levels in the spinal cord of an untreated injury condition (FIG. 11). In addition, a common hallmark of spinal inflammation, microglial activation, was evaluated using labeling for Iba1, and showed that it was also lowered in the superficial dorsal horn at day 7 after treatment with sPLA₂ inhibitor-loaded micelles (FIG. 12). Similar to spinal sPLA₂ expression, Iba1 levels were significantly reduced (p=0.0002) following treatment with sPLA₂ inhibitor-loaded micelles, while Iba1 levels in the untreated spinal cord remain 2.32±1.25-fold over normal levels (FIG. 12).

### Example 5: Therapeutic Efficacy Following Delayed Intravenous Administration of sPLA₂ Inhibitor-Loaded Micelles

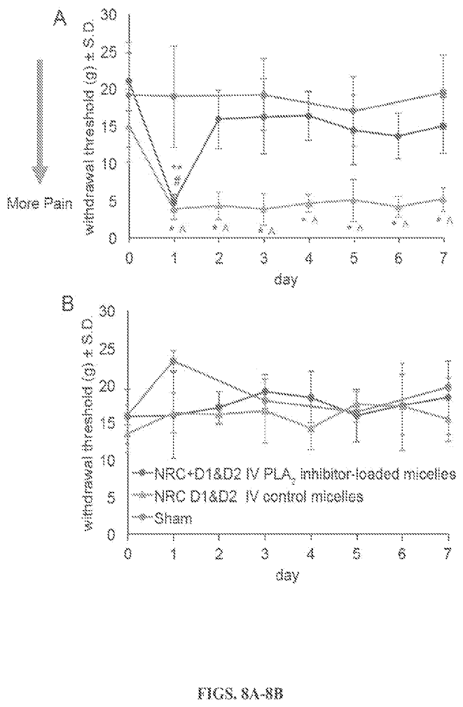

Since local administration of sPLA₂ inhibitor-loaded micelles prevents the onset of pain after nerve root compression (FIG. 7) and seems to modulate spinal inflammation even at later times (FIGS. 11 and 12), it was tested if giving these sPLA₂ inhibitor-loaded micelles via systemic routes after pain has been established would be effective as a treatment to abolish or reduce pain. As such, the sPLA₂ inhibitor-loaded micelles were given via the tail vein on days 1 and 2 post-injury, at the same dose that prevents pain via the local administration after nerve root compression. As expected, the two groups, which received sPLA₂ inhibitor-loaded and unloaded micelles, subjected to painful nerve root compression exhibited a significant reduction (p<0.0053) in withdrawal thresholds compared with pre-injury thresholds as early as day 1 post-injury (FIG. 8A). Since those behavioral assessments are made prior to any micelle treatment, withdrawal thresholds in the unloaded and inhibitor-loaded micelle groups are also significantly lower (p<0.001) compared to the sham surgical control group on day 1.

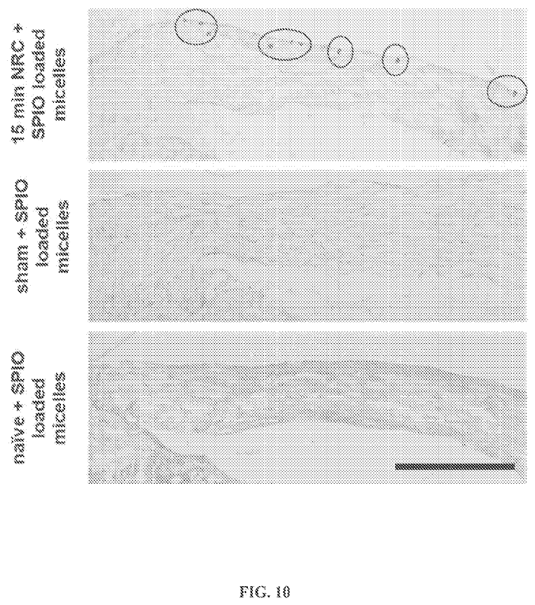

However, after behavioral testing on day 1, micellar treatment with inhibitor was given and the withdrawal threshold increases on day 2 compared to sham and pre-injury levels indicating less pain (FIG. 8A). After the second micellar treatment dose on day 2, withdrawal thresholds are still elevated on day 3 and remain the same as pre-injury through day 7 (FIG. 8A). After sPLA₂ inhibitor-loaded micelle treatment, the responses are significantly increased (*p<0.0024) through day 7 over thresholds in the group receiving a painful nerve root compression and unloaded micelles (FIG. 8A). In fact, there are no differences between the micelle treatment group and sham group on any post-treatment days. However, repeated intravenous administration of control micelles without the inhibitor does not alter the pain response, and thresholds remain significantly lower than baseline (p<0.0001) and sham surgical control responses (p<0.002) (FIG. 8A). Similar to local administration of sPLA₂ inhibitor-loaded micelles (FIG. 7B), the contralateral withdrawal thresholds are not different between the two treatment groups or sham group at any day post-injury (FIG. 8B). In addition, as with the local administration of sPLA₂ inhibitor-loaded micelles (FIG. 9), iron oxide particles localize only to the injured C7 dorsal nerve root after intravenous delivery of the sPLA₂ inhibitor-loaded micelles (FIG. 10), with no evidence of accumulation in the treated sham and naïve C7 nerve roots. These studies validate sufficient selective accumulation to the injury site with systemic delivery.

Systemic treatment with sPLA₂ inhibitor-loaded micelles also significantly reduces (p=0.038) sPLA₂ expression in the spinal dorsal horn compared to treatment with control unloaded micelles at day 7, almost to levels of a normal naïve spinal cord (FIG. 13). Similarly, sPLA₂ expression in the DRG is also significantly (p=0.002) reduced by 2.29±0.39-fold to normal levels, after treatment with sPLA₂ inhibitor-loaded micelles (FIG. 13). Consistent with the histological evidence of micelle accumulation at the site of injury (FIG. 10), decreased sPLA₂ expression in the spinal cord and DRG after systemic micellar treatment further suggests that the sPLA₂ inhibition occurs at the injured C7 spinal level.

### Example 6: Discussion

It was shown herein that sPLA₂ expression after painful injury is significantly increased (FIG. 2). This finding suggests that sPLA₂ can be a unique signature of painful tissue and can act as a novel therapeutic target for neuropathic pain treatment. However, traditional approaches for delivery of anti-inflammatory and neuromodulatory drugs are not very effective for attenuating or abolishing the pain. Drug delivery systems utilizing nanoparticles are increasingly being used to improve therapeutic delivery owing to their ability to increase the effective drug concentration at sites of inflammation while reducing off target toxicity⁴⁵. In this study, the sPLA₂ inhibitor TEA-PC was incorporated into a nanoformulation. These micelle nanoparticles can release their payload based on the pathological conditions by the pain state itself, i.e., different sPLA₂ activity and expression. It was found that leveraging the robust neuroinflammation that occurs with painful neuropathy effectively directs the accumulation and release of encapsulated drugs from micelles and provides long-lasting pain relief (FIG. 7A and FIG. 8A). Capitalizing on increased expression of sPLA₂ in the dorsal root ganglia⁴⁸ ensures localized release of the inhibitor drug where it can have the most impact on phospholipase activity. Administration of these sPLA₂ inhibitor-loaded micelles immediately after compression and directly to the nerve root fully prevents the pain that typically develops after a painful root injury (FIG. 7A). However, these studies were only carried out to post-operative day 7, and it is unknown if pain develops in the following weeks. More clinically-relevant, is the finding that its intravenous delivery after painful neuropathy is already established abolishes existing pain (FIG. 5A). The intravenous dose of the sPLA₂ inhibitor (0.25 mg/mL) used in these micelles was not only comparable to previous intrathecal doses of PLA₂ inhibitors shown to provide pain relief^{49, 50}, but was also approximately 30-times lower than the oral PLA₂ inhibitor dose and 1000-times lower than the COX-2 inhibitor dose^{51, 52} needed to attenuate neuropathic pain⁵³. Further, unlike many pharmacologic neuropathic pain treatments that require continuous and invasive intrathecal delivery or repeated daily dosing^{51, 52}, the sPLA₂ inhibitor-loaded micelles were effective in fully preventing pain with only a single low-dose or only two separate doses to attenuate existing pain. These studies suggest that these targeted drug delivery methods could provide a much-needed solution to the limited efficacy of current neuropathic pain therapeutics. As a platform, sPLA₂ inhibitor-loaded micelles could not only remove the need for invasive treatments but increase the therapeutic index of many pain therapeutics that are currently not used in the clinic due to substantial side effects.

In addition to preventing and abolishing pain, both administration paradigms of sPLA₂ inhibitor-loaded micelles also normalize the spinal sPLA₂ expression at the C7 spinal level (FIGS. 11 and 13), confirming that the active drug is released as observed with the in vitro experiments (FIG. 5). Further, the extent of the decreases in spinal sPLA₂ is similar for both administration paradigms (FIGS. 11 and 13), suggesting that the systemic administration achieved similar inhibitory activity at the injured spinal level to that of locally administered injections. However, since neither the sPLA₂ inhibitor TEA-PC nor the encapsulated TEA-PC, readily cross the blood spinal cord barrier (BSCB)⁵⁴, it may have trafficked there when the barrier was disrupted. BSCB breakdown occurs as early as 1 day after spinal cord and radicular injuries but is restored by day 7. Although, both treatments can leverage the BSCB being open to meditate sPLA₂ in the spinal cord, it is unknown if the same effects would be observed with a later delivery time. Despite this uncertainty, current neuropathic pain treatments such as morphine and cytokine antagonists, even given as early as day 1 and to the injury site or directly to the spinal cord, fail to completely abolish pain^{55, 56}. Whether the encapsulated drug relieves pain by acting exclusively in the periphery, spinally or through a combination of both, the use of PLA₂-responsive phospholipid micelles as a delivery platform ensures the drug's highest possible therapeutic value.

Despite sPLA₂'s established role in neuroinflammation^{49, 57}, few studies have utilized it for pain treatment. Although direct application of sPLA₂ to the naïve spinal cord induces pain⁵⁸, most interventions focus on its downstream inflammatory effects, including blocking the COX enzymes, which utilize arachidonic acid for the synthesis of prostaglandins^{58, 59}. Yet, COX inhibitors and the larger class of non-steroidal anti-inflammatory drugs (NSAIDs) have limited utility for neuropathic pain since they have substantial renal and cardiovascular toxicities and limited efficacy without repeated administration⁶⁰. Further, although other phospholipase A2 inhibitors reduce pain after neuropathy, analgesia is only transient⁵⁰. Intrathecal administration of the cytosolic phospholipase A2 inhibitor (cPLA₂) AACOCF3 reduces behavioral sensitivity when given 1 day after chronic constriction of the sciatic nerve, but only if given under daily repeated dosing⁴⁵. In contrast, as shown herein, pain is abolished immediately after the first treatment and is fully attenuated in a sustained manner by two doses (FIG. 8). The differences in dosing between sPLA₂ and cPLA₂ inhibition may depend on differences between arachidonic acid generation from these two phospholipase A₂ isoforms. Without being bound to any particular theory, in vitro activity studies suggest that cPLA₂ may have more of a regulatory role in cellular membrane maintenance whereas sPLA₂ may be more responsible for arachidonic acid production⁶¹. Inhibiting sPLA₂ may be more potent for mitigating inflammation and providing pain relief, as evidenced by the decreased activation of spinal microglia at day 7 (FIG. 12). Since the increased neuroinflammation evident days after injury is responsible for the progression towards persistent neuropathic pain, it is likely that inhibiting sPLA₂ in this early window interferes with the establishment of persistent pain altogether. Whether a larger concentration of encapsulated TEA-PC or greater administration dose of sPLA₂ inhibitor-loaded micelles on day 1 would provide long-lasting pain relief in a single dose is unknown. Nevertheless, the systemic dose used to inhibit sPLA₂ on days 1 and 2 is sufficient to reduce sPLA₂ expression in the spinal cord and DRG at day 7 (FIG. 13), suggesting that early sPLA₂ inhibition in both regions may be necessary to attenuate pain. While further studies are needed to identify the individual contributions of peripheral and spinal sPLA₂ to the progression of persistent pain and to inform dosing paradigms for patients, these data suggest that systemic sPLA₂ inhibition can be a potent alternative to current anti-inflammatory strategies used to manage persistent neuropathic pain.

This study demonstrates that PLA₂-responsive phospholipid micelles provide localized delivery of encapsulated drugs and can even be more efficacious than traditional intrathecal delivery methods for treating chronic pain. Leveraging sPLA₂ activity in painful syndromes helps exploit the release of this, and possibly other, drugs to modulate cascades involved in pain, leading to pain treatment and possible imaging tracking of the disease state. Given the role of sPLA₂ in many different inflammatory and neuropathic pain syndromes^{57, 62}, PLA₂-responsive phospholipid micelles have a potentially broad reach. Further, this PLA₂ micelle platform can be expanded to encapsulate other drugs including neuromodulatory drugs, which despite success in animal models, have not been adopted into the clinic since systemic administration requires high doses which induce substantial off-target side effects. PLA₂ inhibitor loaded phospholipid micelles can address the clinical challenge of delivering pain therapeutics to achieve long lasting pain relief, without the debilitating off-target toxicities.

### Materials and Methods for Examples 7-13

Materials

1-Palmitylthio-2-palmitoylamido-1,2-dideoxy-sn-glycero-3-phosphorylcholine (thioetheramide-PC) was purchased from Cayman Chemical (Ann Arbor, Mich.). Hydrogenated soy phosphatidylcholine (HSPC), 1,2-distearoyl-sn-glycero-3-phosphoethanolamine-N-[methoxy(polyethylene glycol)-2000] (DSPE-PEG2000), 1,2-dioleoyl-3-trimethylammonium-propane (DOTAP), 1,2-dioleoyl-sn-glycero-3-phosphoethanolamine-N-(Lissamine rhodamine B sulfonyl) (Rhod-PE), 1,2-distearoyl-sn-glycero-3-phosphoethanolamine-N-[amino(polyethylene glycol)-2000]-N-(Cyanine 7) (DSPE-PEG2000-Cy7), and 1-palmitoyl-2-{6-[(7-nitro-2-1,3-benzoxadiazol-4-yl)amino]hexanoyl}-sn-glycero-3-phosphocholine (NBD-PC) were purchased from Avanti Polar Lipids, Inc. Secreted phospholipase A2 (PLA2) enzyme from Naja mossambica was purchased from Sigma-Aldrich Co. Rabbit polyclonal antibody to sPLA2-IIa, phospho-P65 (p-P65) and phospho-P100 (p-P100) antibodies were purchased from Abcam. All other chemicals were used as received. All of the buffer solutions were prepared with deionized water.

### Synthesis of sPLA₂i-Loaded Phospholipid Nanoparticles

sPLA2i-NPs were prepared by hydration of dry sPLA2i/lipid films. A mixture containing 10 mol % DOTAP/25 mol % thioetheramide-PC/65 mol % DSPE-PEG2000 was prepared in a round bottom flask. The total amount of thioetheramide-PC was 0.25 mg. For the preparation of fluorescently labeled nanoparticles, a small amount (1 mol %) of the fluorescent lipids, Rhod-PE or DSPE-PEG2K-Cy7, was also added to the thioetheramide-PC/lipid mixture. The solvent was removed using a direct stream of nitrogen prior to vacuum desiccation for a minimum of 4 hours. Nanoparticles were formed by adding an aqueous solution (0.1×PBS, pH=7.4) to the dried film and incubating in a 25° C. water bath for 5 minutes and then vortexing for another 3 minutes. The resulting solution was then centrifuged at 3000 g for 5 minutes to remove the large aggregates. Finally, the nanoparticles were filtered through a 0.22 μm cellulose acetate membrane filter (Nalgene, Thermo Scientific) and stored in the dark at 4° C. Control nanoparticles, including nanoparticles without sPLA2i (10 mol % DOTAP/25 mol % HSPC/65 mol % DSPE-PEG2000) and nanoparticles without DOTAP (10 mol % HSPC/25 mol % sPLA2i/65 mol % DSPE-PEG2000), were prepared using the similar procedures as above.

Synthesized sPLA2i-NPs were incubated in water at 4° C. as well as in bovine synovial fluid at 37° C. for the stability study. The diameter and zeta potential of the nanoparticles were measured with dynamic light scattering (DLS, Malvern, Zetasizer, Nano-ZS). The morphology of the nanoparticles was observed using a transmission electron microscope (TEM) (JOEL 1010) by the negative-staining technique.

In Vitro sPLA2 Response Study

To study in vitro sPLA2 response of sPLA2i-NPs, the NBD-incorporated liposomes were incubated with sPLA2i-NPs in the presence of sPLA2 enzyme and the dequenching of NBD fluorescence signal was monitored. To prepare NBD-liposomes, stock solutions of HSPC and NBD-PC in chloroform were mixed in the following molar ratios: HSPC/NBD-PC (80:20). The total amount of HSPC was 1 mg. The solvent was removed using a direct stream of nitrogen prior to vacuum desiccation for a minimum of 4 hours. 0.2 ml deionized water was then added to the dried lipid film and incubated in a 50° C. water bath for 0.5 hours and then sonicated for another 30 minutes. The stock solution of NBD-incorporated phospholipid liposomes was stored in the dark at 4° C.

Dequenching measurements were performed by first preincubating a mixture of either sPLA2i-NPs or Ctrl-NPs (without sPLA2i) with the sPLA2 enzyme (10 μL sPLA2i-NPs [sPLA2i: 0.25 mg ml-1] or Ctrl-NPs+6.67 μL sPLA2 enzyme [sPLA2: 7.5 U ml-1]) for 20 minutes. Fluorescence measurements of the NBD-incorporated liposomes in buffer (20 μL of NBD-liposomal suspension ([HSPC]=1 mg ml-1)+0.48 ml of 10 mM HEPES (pH 7.4) buffer solution containing 2 mM CaCl₂) were taken for 5 minutes prior to the addition of the incubation mixture of the sPLA2i-NPs or Ctrl-NPs. The fluorescence intensity at 520 nm was measured on a SPEX FluoroMax-3 spectrofluorometer (Horiba Jobin Yvon) using an excitation at 460 nm. The amount of NBD dequenched (% NBD dequenched) was calculated by means of Equation 1% NBD dequenched=([Ix−I0]/[It−I0])×100

where I0 is the fluorescence intensity of the liposomal suspension containing NBD at the initial time, Ix is the fluorescence intensity at any given time, and It is the fluorescence intensity after addition of 20 μL Triton X-100 (50 mM) to the suspension at the end of experiment.

Cell Culture

Primary mouse chondrocytes were isolated from the distal femoral and proximal tibial epiphysis of mice (3-6 days old) via enzymatic digestion. Briefly, tissues were incubated with 0.25% trypsin (Invitrogen) for 1 h, followed by 2 h digestion with 300 U ml-1 type I collagenase (Worthington Biochemical). Dissociated cells were seeded in culture plates, and attached cells were considered primary mouse chondrocytes. They were cultured in DMEM/F12 containing 10% FBS, 100 μg ml-1 streptomycin, and 100 U ml-1 penicillin. Primary mouse chondrocytes between passage 0 and passage 3 were used for MTT assay experiments. m,m,

MTT Assay

Primary mouse chondrocytes (5000 cells per well) were seeded in 96-well plates and incubated overnight (37° C., 5% CO2) to allow the cells to attach to the surface of the wells. The sPLA2i-NPs were added to wells at five different sPLA2i concentrations ranging from 0.625 to 10 μg ml-1 (0.625, 1.25, 2.5, 5, 10 μg ml-1), and the cell viabilities were determined according to the supplier's instructions. After 24 hours incubation, the medium containing nanoparticles in each well was aspirated off and replaced with 100 μL fresh medium and 10 μL of MTT reagent. The cells were incubated for 2 to 4 hours. Then, 100 μL detergent reagent was added and left at room temperature in the dark for 4 hours. Finally, the absorbance of formazan product was measured on a Tecan microplate reader (BioTek Instruments, Inc) at 570 nm. Cell viability was calculated using the following equation: Cell viability (%)=(Absorbancesample/Absorbancecontrol)×100.

Bovine Cartilage Explant Harvest and Culture

Young (1-2 weeks old) bovine knee joints were obtained from Vendors (Lampire biological laboratories), and cartilage explants were harvested from the trochlear groove using biopsy punch and cultured with chemically defined medium (DMEM, 1% ITS+Premix, 50 μg ml-1 L-proline, 0.104 dexamethasone, 0.9 mM sodium pyruvate and 50 μg ml-1 ascorbate 2-phosphate) in 48-well plate.

Bovine Cartilage Explant Penetration Assay

Bovine cartilage explants (6 mm in diameter and 2 mm in thickness) were incubated with free rhodamine, rhodamine-labeled sPLA2i-NPs (DOTAP−) or sPLA2i-NPs (DOTAP+) in 500 μl of culture medium for 48, 96, 144 or 192 hours at 37° C. and 5% CO2 under gentle agitation. Culture medium with free rhodamine, rhodamine-labeled sPLA2i-NPs (DOTAP−) or sPLA2i-NPs (DOTAP+) was replaced every other day. In all cases, the final Rhodamine concentration in the culture medium was 10 μM. After incubation, cartilage explants were washed three times with PBS, fixed with 4% Paraformaldehyde (PFA), dehydrated with 20% sucrose+2% PVP (Polyvinylpyrrolidone) followed by embedding with 30% sucrose+2% PVP+8% Gelatin. Sections were mounted with DAPI Fluoromount-G Mounting Medium on glass slides and immediately observed under confocal microscope (Zeiss LSM 710). Quantitative analysis was performed on maximum intensity projections of Z-stack images taken from 100 μm thick sections.

Bovine Cartilage Explant Uptake Assay Sdfdf

A total volume of 300 μL of free rhodamine, rhodamine-labeled sPLA2i-NPs (DOTAP−) or sPLA2i-NPs (DOTAP+) in culture medium was added to bovine cartilage explants (3 mm in diameter and 2 mm in thickness). The final rhodamine concentration in the culture medium was 10 μM. The explants were incubated for 24 hours at 37° C. and 5% CO2 under gentle agitation. The explants were then removed from the medium, washed tree times with PBS, imaged by IVIS (Spectrum, PerkinElmer). Radiant efficiency within a fixed anatomical region of interest (ROI) was measured using Living Image software.

In Vivo Joint Retention Assay

The mouse knee joints retention assay was assessed by intra-articular injection of 10 μl of 10 μM free ICG or 10 μM Cy7 doped sPLA2i-NPs in healthy (3 months old) and OA (8 weeks post DMM surgery) mouse knees. The rat knee joints retention assay was assessed by intra-articular injection of 40 μl of 10 μM free ICG or 10 μM Cy7 doped sPLA2i-NPs in healthy rat knees (3 months old). An IVIS (Spectrum, PerkinElmer) was used to serially acquire fluorescence images within each joint over a period of 4 weeks. Using Living Image software, radiant efficiency within a fixed anatomical region of interest (ROI) was measured.

In Vivo Biodistribution Assay

vivo biodistribution study was performed by intra-articular injection of 10 μl of PBS or 10 μM Cy7 doped sPLA2i-NPs in mouse knees (3 months old). At 24 hours or 1 month following injection, the mice were sacrificed. The knee joints, blood, and major organs (heart, liver, spleen, lung, kidney) were harvested. Knees were dissected to isolate the major joint components, including the surrounding tissues (quadriceps, patella, patellar ligament, synovium, fat pad), femoral condyles, tibial plateau and meniscus. All the major joint components, blood and organs were imaged using the IVIS, and the data was analyzed as described above.

Mouse Femoral Head Explants Penetration Assay

Mouse femoral heads were collected from 8-week-old male mice and cultured for 48 hours in chemically defined medium in 48-well plate. Following the culture, mouse femoral head explants were stimulated using 10 ng ml-1 recombinant mouse IL-1β (PeproTech) for 2 days. On day 3, rhodamine-labeled sPLA2i-NPs were incubated with the IL-1β-stimulated femoral heads for 24 or 48 hours at 37° C. with gentle agitation. The final rhodamine concentration in the culture medium was 10 μM. Following incubation, femoral heads were washed with PBS, fixed with 4% PFA, dehydrated with 30% sucrose and embedded in Tissue-Tek OCT Compound. 6-μm-thick cyrosections were cut and mounted with DAPI Fluoromount-G Mounting Medium on the slides and imaged with a fluorescence microscope (Nikon, Eclipse 90i). Images were analyzed with ImageJ to quantify the penetration depth nanoparticle into the cartilage. Fluorescence intensity within each image was measured with ImageJ and normalized to the fluorescence of the outermost cartilage surface of the treated femoral head. Dasds

Mouse Femoral Head Explants Degradation Assay

Mouse femoral heads were collected using the same procedure as described in mouse femoral head explants penetration assay. To test the therapeutic effects of sPLA2i-NPs, femoral head cartilage explants were divided into 4 groups to receive PBS (i.e. untreated), IL-1β, IL-1β and Ctrl-NP, and IL-1β and sPLA2i-NP treatments, respectively, for 8 days. The final IL-1β and sPLA2i concentration in the culture medium was 10 ng ml-1 and 0.1 mg ml-1, respectively. The culture medium was replaced at day 2, 4, and 6. After 8-day incubation, mouse femoral heads were then fixed with 4% paraformaldehyde overnight followed by decalcification in 0.5 M EDTA (pH=7.4) for 2 weeks and processed for 6-μm paraffin sections. Paraffin sections were used for safranin-O staining, and the safranin-O-positive area was quantified with ImageJ. OA severity was evaluated by Mankin score. Both quantifications were based on the section with the most severe loss of safranin-O staining and cartilage damage.

Quantitative RT-PCR

Mouse femoral heads were collected and treated using the same procedure in femoral head explants degradation assay. Total RNA was harvested from the femoral head articular cartilage using Tri Reagent (Sigma). Taqman Reverse Transcription kit (Applied BioSystems) was used to reverse-transcribe mRNA into cDNA. Following this, PCR was performed using a Power SYBR Green PCR MasterMixkit (Applied BioSystems). The primer sequences for the genes used in this study are listed in Table 1 below.

TABLE 1
Mouse real-time PCR primer sequences used in this study.

| | | SEQ ID | | SEQ ID |
|---|---|---|---|---|
| Gene | Forward primer | NO: | Reverse primer | NO: |
| SPLA2 | 5′-TGGCCTTTGGCTCAATAC-3′ | 1 | 5′-GGCATCCATAGAAGGCATAG-3′ | 7 |
| Aggrecan | 5′-CTACCGCTGTGAAGTGATG-3′ | 2 | 5′-GGTGTAGCGTGTGGAAATAG-3′ | 8 |
| Col2a1 | 5′-CAAGAACAGCAACGAGTACCG-3′ | 3 | 5′-GTCACTGGTCAACTCCAGCAC-3′ | 9 |
| Mmp13 | 5′-TGACCTCCACAGTTGACAGG-3′ | 4 | 5′-ATCAGGCACTCCACATCTTGG-3′ | 10 |
| Adamts5 | 5′-GCATCCCAGCATTAGGAATTCA-3′ | 5 | 5′-GGTGAGAGCTGCATTGGAGGTA-3′ | 11 |
| ß-actin | 5′-TCCTCCTGAGCGCAAGTACTCT-3′ | 6 | 5′-CGGACTCATCGTACTCCTGCTT-3′ | 12 |

Human Articular Cartilage Samples

The human OA articular cartilage samples were prepared from the de-identified specimens obtained at the total arthroplasty of the knee joints and used for immunohistochemical examination for sPLA2. The serial sections were stained by Safranin O and Fast green staining to evaluate OA phenotype and stage.

Animal Care

In accordance with the standards for animal housing, mice were group housed in an atmosphere of 23-25° C. with a 12-hour light/dark cycle, and allowed free access to water and standard laboratory pellets.

To induce mouse OA that mimics chronic OA in human patients, male mice at 3 months of age were subjected to DMM surgery at right knees and sham surgery at left knees. Briefly, in DMM surgery, the joint capsule was opened immediately after anesthesia and the medial meniscotibial ligament was cut to destabilize the meniscus without damaging other tissues. In Sham surgery, the joint capsule was opened in the same fashion but without any further damage.

To induce mouse OA that is noninvasive and mimics post-traumatic OA in human patients, male mice (2 months old) were subjected mechanic loading at the right knees and sham loading at left knees. Briefly, under anesthesia, the right tibiae were positioned with the knee downward in deep flexion between custom-made cups and subjected to axial compressive loads with a peak force of 6 or 9 Newtons (N) with a 0.5 N preload force to maintain the limb in position between loading cycles. Cyclic loads were applied for 0.34 s with a rise and fall time each of 0.17 s and a baseline hold time of 10 s between cycles for 60 cycles. The uninjured left knees were used as controls.

Nanoparticle Administration

For treatment, nanoparticles were administrated using sterile techniques: the right knees were kept in a flexed position and a total volume of 10 μl of PBS, sPLA2i (0.25 mg ml-1), Ctrl-NPs, or sPLA2i-NPs (sPLA2i: 0.25 mg ml-1) was injected intra-articularly with a 30-gauge needle. For DMM surgical OA model, the first injection was performed immediately after surgery. Injections were then repeated every 2 weeks for 2 or 4 months. In total, there are 4 injections for the 2 months group and 8 injections for 4 months group. For load-induced OA model, injections (i.a.) were performed immediately and at 48 hours after loading. There are 2 injections in total for each mouse in this model.

After 2 months treatment for DMM mice, some major organs (kidney, liver, lung, heart, and spleen) and blood were collected. Tissue sections were stained with hematoxylin and eosin (H&E) to assess the effects of different treatment on mouse organ morphology. The blood indexes were measured after receiving 2-month treatment of PBS, sPLA2i, Ctrl-NPs, or sPLA2i-NPs.

Histology

After euthanasia, mouse knee joints were harvested and fixed in 4% paraformaldehyde overnight followed by decalcification in 0.5 M EDTA (pH 7.4) for 4 weeks prior to paraffin embedding. A serial of 6 μm-thick sagittal sections (about 100) were cut across the entire medial compartment of the joint until ACL junction. To measure the thicknesses of articular cartilage and chondrocyte numbers, 3 sections from each knee, corresponding to ¼ (sections 20-30), 2/4 (sections 45-55), and ¾ (sections 70-80) regions of the entire section set, were stained with Safranin 0/Fast green and quantified using BIOQUANT software. The final measurement is an average of these three sections. We defined uncalcified cartilage as the area from articular surface to the tide mark and calcified cartilage as the area from tide mark to cement line. The method to measure Mankin Score was described previously(37). Briefly, two sections within every consecutive six sections in the entire section set for each knee were stained with Safranin 0/Fast green and scored by two blinded observers. Each knee received a single score representing the maximal score of its sections.

Synovitis score grading was carried out in 6-μm paraffin sections of sagittal mouse knee sections stained with H&E. The following basic morphological parameters of synovitis were included: (i) hyperplasia/enlargement of synovial lining layer, (ii) degree of inflammatory infiltration and (iii) activation of resident cells/synovial stroma, including fibroblasts, endothelial cells, histiocytes, macrophages, and multinucleated giant cells. All parameters are graded from 0 (absent), 1 (slight), 2 (moderate) to 3 (strong positive).

Cartilage injury length was measured in Safranin 0/Fast green stained paraffin sections. From the serial Safranin-0 stained sections in each sample, we selected one section with the widest cartilage lesion that featured by focal loss of Safranin-0 staining, minor fissuring of articular cartilage, and atrophy of articular chondrocytes. According to these histological changes, it is possible to identify the demarcation between the normal and injured cartilage tissue, and the length of the cartilage lesion range was measured.

Paraffin sections were used for immunohistochemistry and TUNEL assay. For mouse samples, after appropriate antigen retrieval, slides were incubated with primary antibodies, such as rabbit anti-sPLA2-IIA (Abcam, ab23705), rabbit anti-p-P65 (Abcam, ab86299), rabbit anti-p-P100 (Abcam, ab194919) at 4° C. overnight, followed by binding with biotinylated secondary antibodies and DAB color development. The TUNEL assay was carried out according to the manufacturer's instructions (Millipore, s7101). For human samples, anti-sPLA2-IIA (Abcam, ab23705) antibody was used.

OA Pain Analysis

The knee joint pain after DMM surgery or loading injury was evaluated in mice weekly before and after surgery using von Frey filaments as described previously(38). An individual mouse was placed on a wire-mesh platform (Excellent Technology Co.) under a 4×3×7 cm cage to restrict their move. Mice were trained to be accustomed to this condition every day starting from 7 days before the test. During the test, a set of von Frey fibers (Stoelting Touch Test Sensory Evaluator Kit #2 to #9; ranging from 0.015 to 1.3 g force) were applied to the plantar surface of the hind paw until the fibers bowed, and then held for 3 seconds. The threshold force required to elicit withdrawal of the paw (median 50% withdrawal) was determined five times on each hind paw with sequential measurements separated by at least 5 minutes.

Micro-Computed Tomography (microCT) Analysis

The distal femur of mouse knee joints was scanned at a 6-μm isotropic voxel size with a microCT 35 scanner (Scanco Medical AG, Brüttisellen, Switzerland). All images were smoothened by a Gaussian filter (sigma=1.2, support=2.0). Measurement of SBP thickness was described previously(39). Briefly, sagittal images were contoured for the SBP followed by generating a 3D color map of thickness for the entire SBP along with a scale bar. This map was then converted to a grayscale thickness map. The region of interest (ROI) was circled and the average SBP thickness within ROI is calculated by average grey value/255*max scale bar value. Coronal images were also contoured for the osteophyte followed by 3D reconstruction and volume calculation.

Statistical Analysis

Data are expressed as means±standard error (s.e.m) and analyzed by t-tests, one-way ANOVA with Dunnett's or Turkey's posttest and two-way ANOVA with Bonferroni's or Turkey's post-test for multiple comparisons using Prism 8 software (GraphPad Software, San Diego, Calif.). For assays using primary chondrocytes and bovine cartilage explants, experiments were repeated independently at least three times and representative data were shown here. Values of p<0.05 were considered statistically significant.

### Example 7: sPLA2 Amount in OA Cartilage

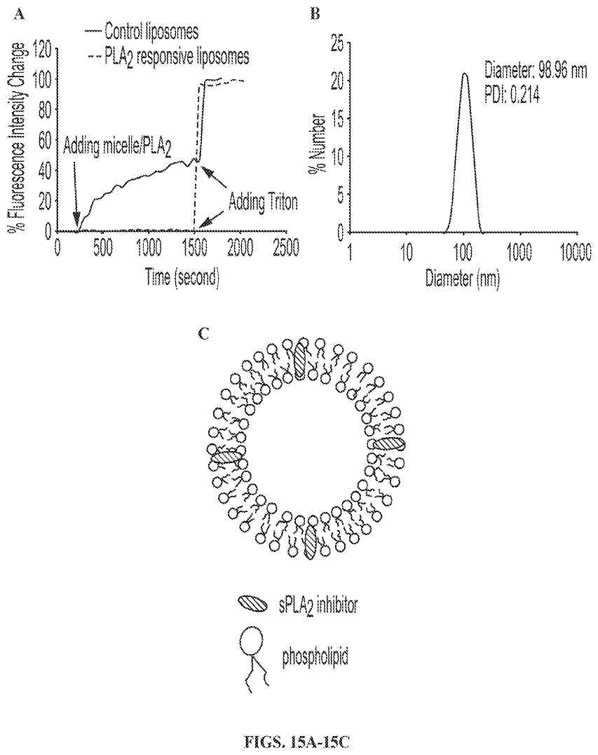

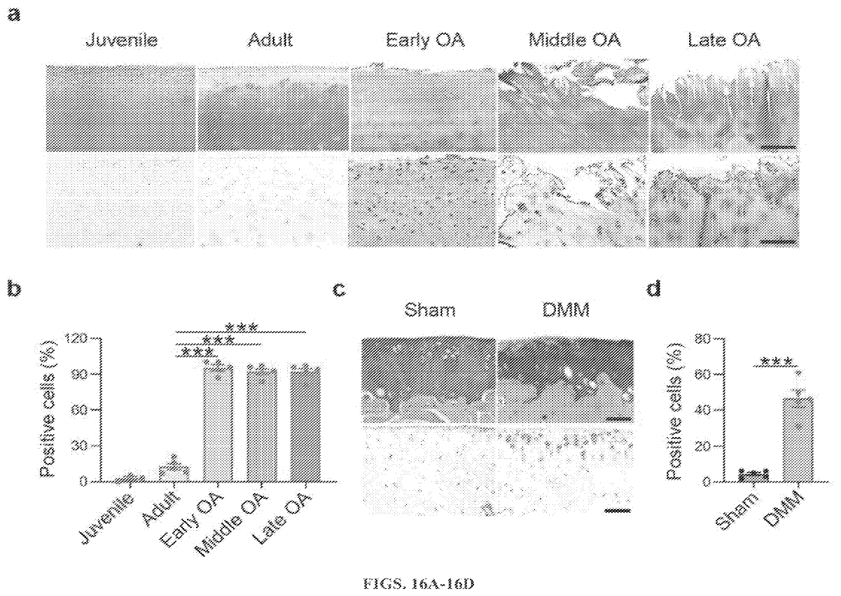

To confirm the elevated levels of sPLA2 enzyme in OA-related tissues, we performed immunohistochemistry (IHC) to investigate a commonly studied isoform sPLA2-IIA in OA and normal knees. In healthy young and adult human cartilage, the level of sPLA2-IIA was very low (FIG. 16a,b). However, in OA cartilage, it was drastically elevated after OA initiation and remained at a high level at early-, middle-, and late-stages of OA progression. Abundant staining was found in the cartilage matrix as well as chondrocytes. Mouse cartilage showed similar results (FIG. 16c,d). While normal mouse articular cartilage displayed only a weak staining of sPLA2-IIA, cartilage in mouse knees receiving destabilization of the medial meniscus (DMM) surgery a month earlier showed greatly elevated staining throughout the cartilage. The marked increase of sPLA2 in human and mouse OA cartilage suggested its potential role in OA development.

### Example 8: sPLA2i-NP Synthesis and Characterization

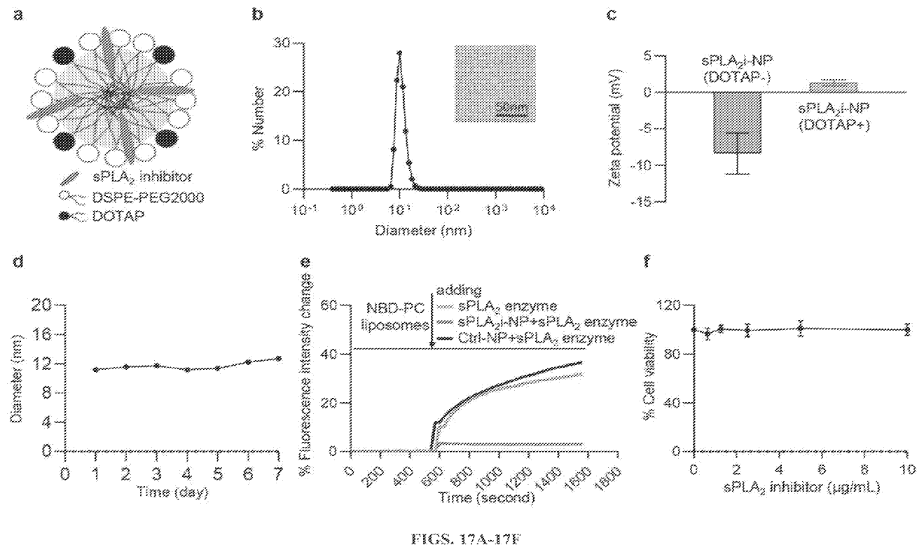

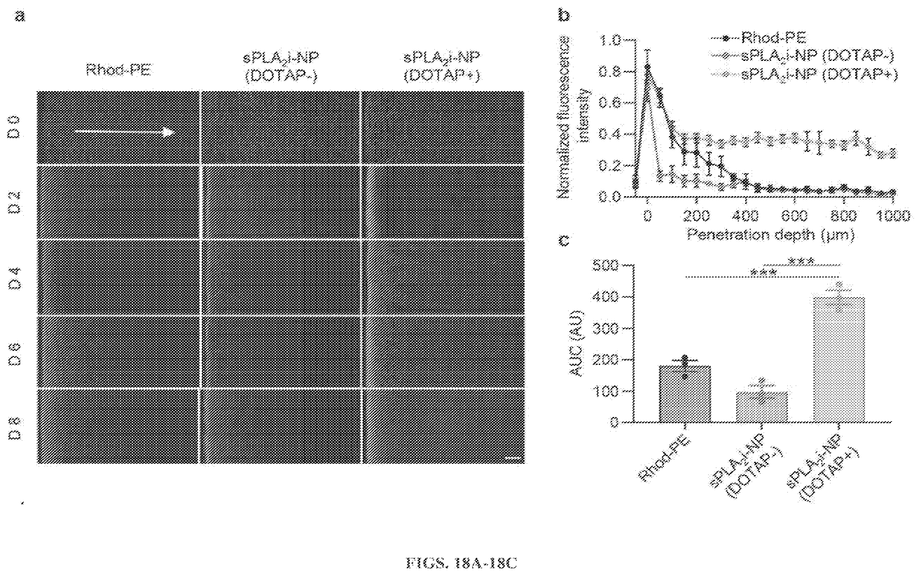

sPLA2i-NPs were prepared by incorporating 25 mol % thioetheramide-PC into phospholipid micelles with lipid composition 10 mol % 1,2-dioleoyl-3-trimethylammonium propane (DOTAP)/65 mol % 1,2-distearoyl-sn-glycero-3-phosphoethanolamine-N-[methoxy(polyethylene glycol)-2000] (mPEG2000-DSPE) (FIG. 17a). Due to its amphiphilic nature, lipid-based sPLA2i was easily doped into the phospholipid film during nanoparticle preparation. Dynamic light scattering (DLS) measurements revealed that sPLA2i-NPs possessed a mean hydrodynamic diameter of 10 nm and a relatively narrow size distribution (FIG. 17b). sPLA2i-NPs observed by transmission electron microscopy (TEM) were approximately spherical in shape together with some worm-like structures. As shown in FIG. 17c, the presence of cationic DOTAP within sPLA2i-loaded nanoparticles transitioned nanoparticles from a negative surface charge (−8 my) to a positive surface charge (+2 my), to increase the retention and penetration ability of sPLA2i-NPs within the cartilage via electrostatic interactions between the nanoparticles and the anionic glycosaminoglycans (GAGs) in the cartilage. The stability of the sPLA2i-NPs was evaluated in water and bovine synovial fluid. There was no observable change in the hydrodynamic diameter of sPLA2i-NPs in water for at least 1 week (FIG. 17d) or in bovine synovial fluid for 24 hours (FIG. 22). ASDASD

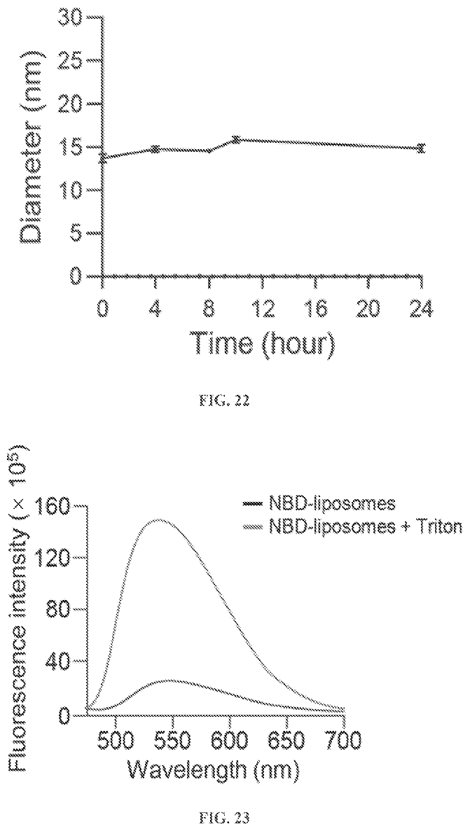

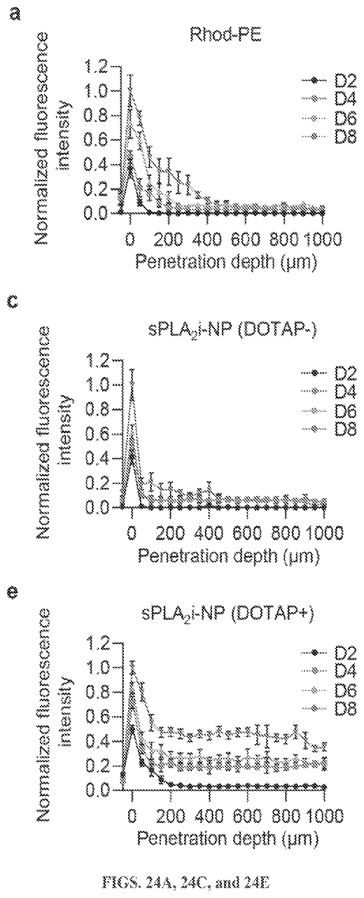

To examine whether sPLA2i-NPs can inhibit sPLA2 enzyme, a fluorescence dequenching assay with liposomes that contain a self-quenching concentration of the fluorescent lipid was used(30). Phospholipid hydrogenated soy phosphatidylcholine (HSPC) liposomes doped with 20 mol % fluorescent lipid 1-palmitoyl-2-{6-[(7-nitro-2-1,3-benzoxadiazol-4-yl)amino]hexanoyl}-sn-glycero-3-phosphocholine (NBD-PC) were prepared in which the NBD fluorescence was self-quenched (FIG. 23). No significant change in fluorescence intensity was observed when the preincubation mixture of sPLA2i-NPs and sPLA2 enzyme was added into NBD-incorporated liposomal suspension. However, a significant increase in the fluorescence intensity was observed upon the addition of the preincubation mixture of Ctrl-NPs (i.e., nanoparticles without sPLA2i) and sPLA2 enzyme (FIG. 17e). These results suggest that sPLA2i-NPs are sPLA2-responsive and can inhibit sPLA2 enzyme.

The cytotoxic effects of sPLA2i-NP were examined in a MTT cell proliferation assay. Specifically, various concentrations of sPLA2i-NPs were incubated with primary mouse chondrocytes for 24 hours. The cell viability for each group was normalized to a control group that was not incubated with any sPLA2i-NPs. In general, sPLA2i-NPs had little effect on the viability of cells up to a sPLA2i concentration of 10 ug/mL (FIG. 170, suggesting their physiological biocompatibility.

### Example 9: sPLA2i-NP Penetration

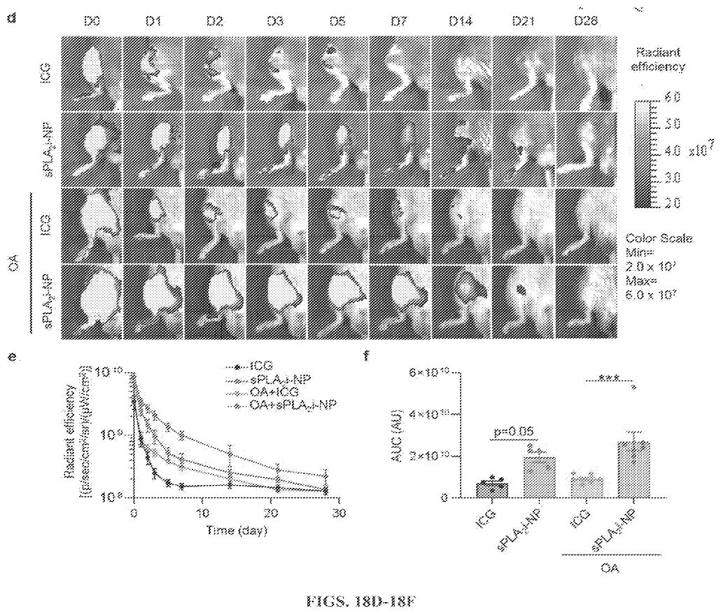

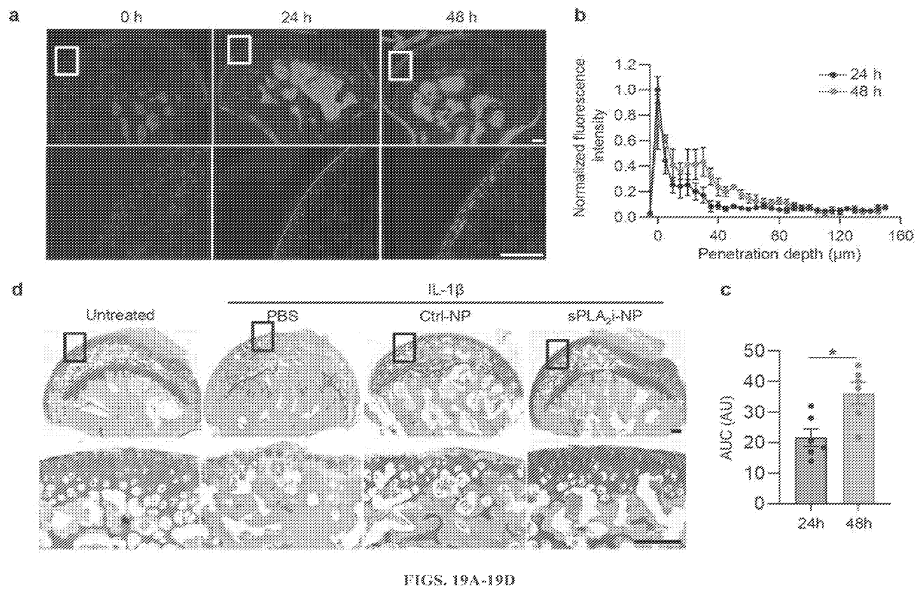

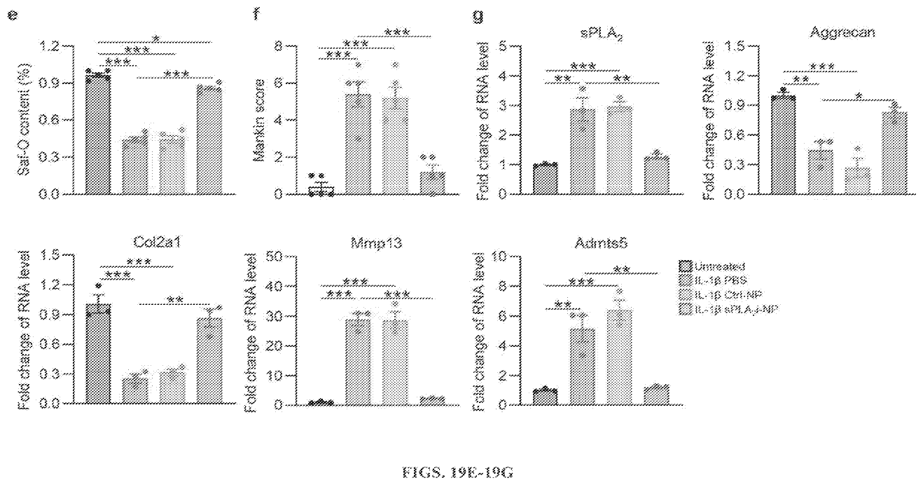

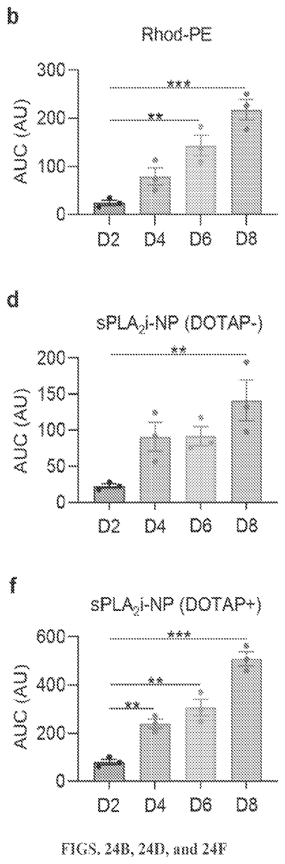

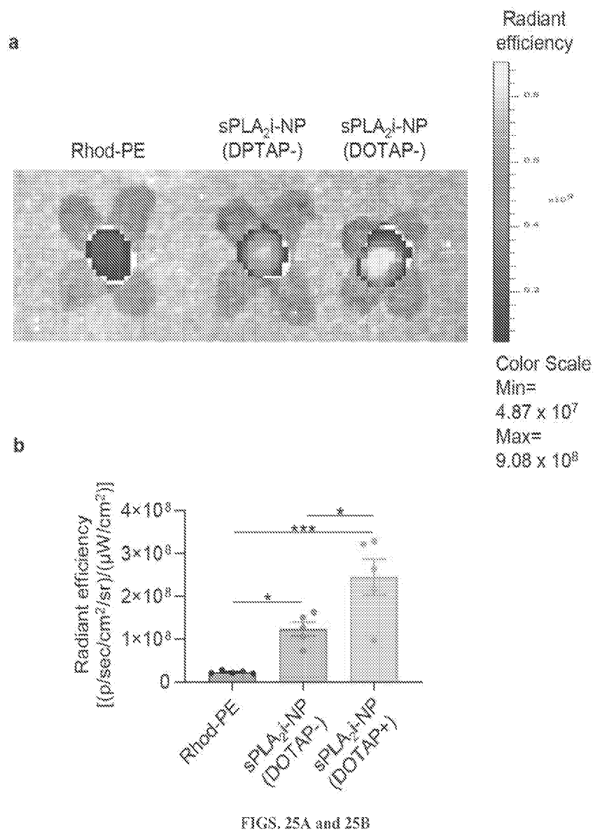

To examine whether the sPLA2i-NPs could penetrate into deep layer of cartilage tissue, sPLA2i-NPs were incubated with bovine cartilage tissue ex vivo. Confocal fluorescence images of cartilage section were acquired preincubation and at various time points after incubation with sPLA2i-NPs (FIG. 18a). In the preincubation images, there was little intrinsic tissue fluorescence. At two days following incubation with the sPLA2i-NPs, a strong fluorescence signal was observed within the superficial zone of cartilage. The fluorescence signal was distributed in the entire cartilage tissue (1-2 mm thickness) as early as day 4, and became stronger over time, indicating the sPLA2i-NPs indeed moved into the deep zone of the cartilage. Control experiments to demonstrate the high penetration capability of sPLA2i-NP were performed by incubation of bovine cartilage explants with sPLA2i-NPs that were not doped with the positively-charged DOTAP. In these tissues, a strong fluorescence signal was only observed within the superficial zone of cartilage, even up to 8 days. Similarly, when bovine cartilage explants were incubated with free fluorescent dye rhodamine, very little fluorescence signal was observed in the deep zone of cartilage. Quantitative analysis of fluorescence images revealed that DOTAP-doped sPLA2i-NPs exhibited a great improvement in cartilage penetration at all time points compared with non-DOTAP doped sPLA2i-NPs or free rhodamine (FIG. 18b and FIG. 24). In addition, the area under curve (AUC) in the cartilage achieved by sPLA2-NPs (DOTAP+) was much larger than that of non-DOTAP doped sPLA2-NPs or free rhodamine (FIG. 18c and FIG. 24). These results indicate that the DOTAP-doped sPLA2i-NPs are able to penetrate into the deep zone of articular cartilage and exhibit high cartilage accumulation.

To further assess the total accumulation of sPLA2i-NPs within cartilage, fluorescence images of bovine cartilage were acquired 24 hours following the incubation of DOTAP-doped sPLA2i-NPs, non DOTAP-doped sPLA2i-NPs, or free rhodamine. As expected, the fluorescent intensity of DOTAP-doped sPLA2i-NPs in the cartilage tissue was significantly higher than that of non-DOTAP doped sPLA2i-NPs or free rhodamine (FIG. 25a,b). This convincingly demonstrates the benefit of including DOTAP moieties in the sPLA2i-NPs. Due to the ability to penetrate cartilage, the DOTAP-doped sPLA2i-NPs were chosen for subsequent studies.

### Example 10: sPLA2i-NP Retention

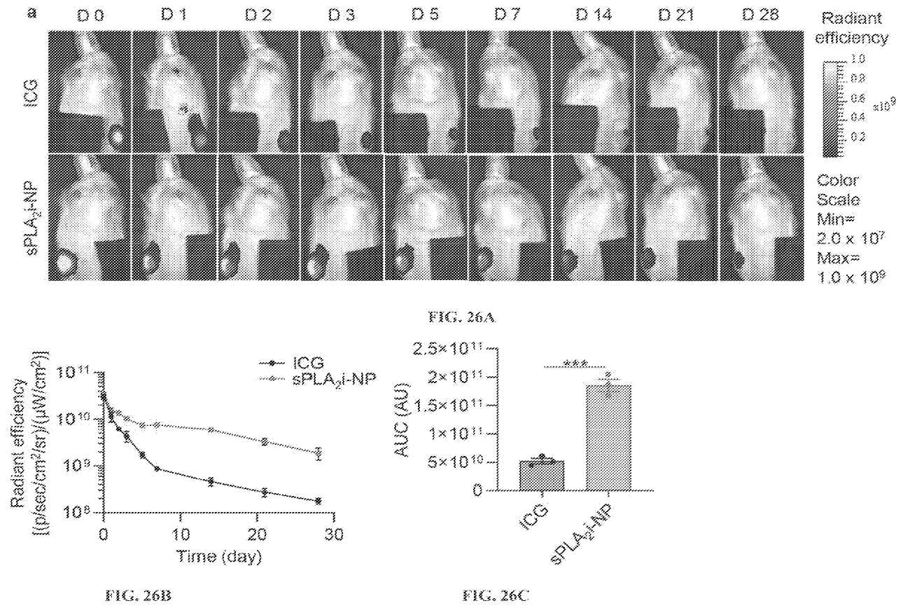

The retention of Cy7-labeled sPLA2i-NPs in the knee joints post intra-articular injection was monitored using in vivo fluorescence imaging. To show the difference in retention between nanoparticles and small-molecule agents, a low molecular weight fluorescent dye indocyanine green (ICG, MW 775) was used for comparison. Their retention within the joints were evaluated in the knee under healthy and early OA conditions. Fluorescence images of mouse knee joints were acquired at various time points post sample injection (FIG. 18d). With a single intra-articular injection, the fluorescence intensity of sPLA2i-NPs in joints was significantly higher than that of free ICG at all time points, indicating that the retention time of sPLA2i-NPs in joints is much longer than that of free ICG. Quantitative analysis of fluorescence images showed that the retention of sPLA2i-NPs in joints with OA condition was even more efficient than that of healthy joints (FIGS. 18e,f). A longer retention time of sPLA2i-NPs over small molecules within knee joints was also confirmed when rats were used in this study (FIG. 26).

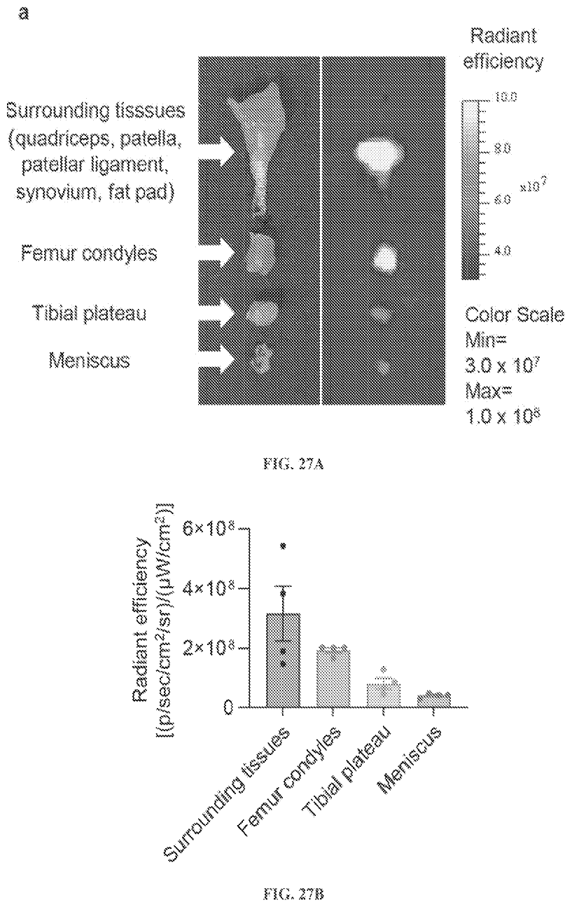

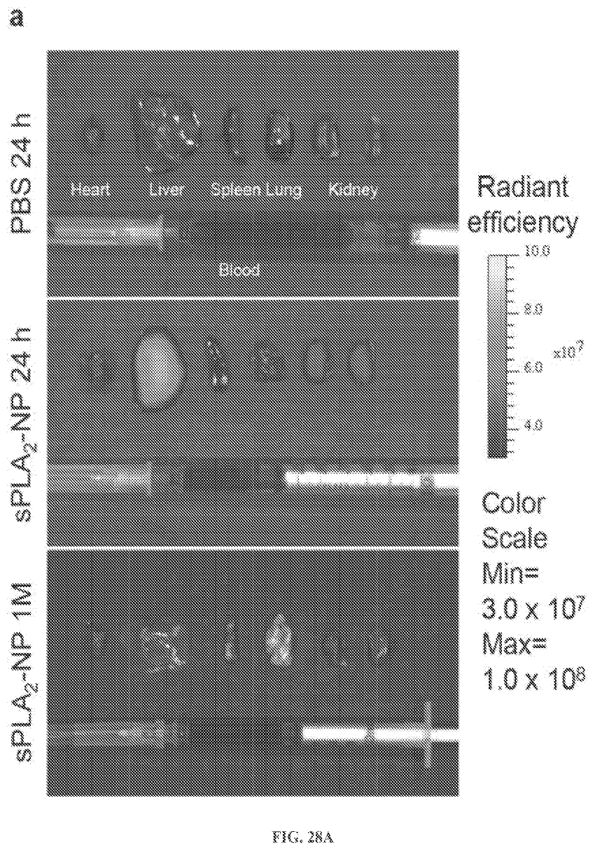

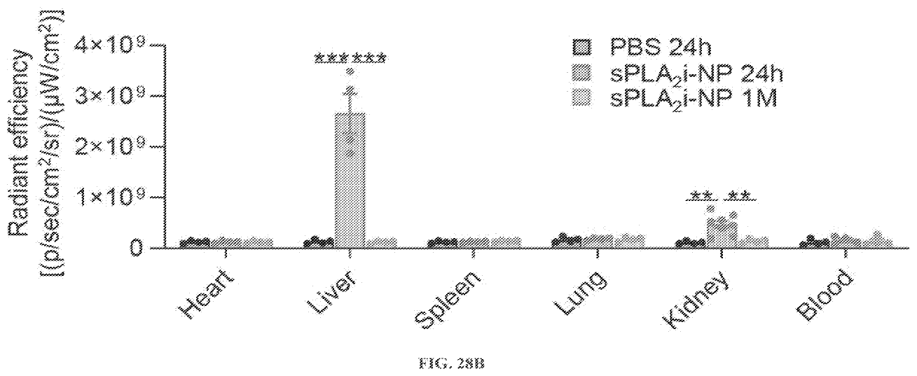

We also examined the biodistribution of sPLA2i-NPs in joint components, internal organs, and blood. At 24 hours post injection, fluorescence signals were detected on the cartilage surfaces of patellar, femur condyles and tibiae plateau as well as on the meniscus (FIGS. 27a,b). By 24 h postinjection, the accumulation of the sPLA2i-NPs was mainly observed in the liver and kidney, but no signal was detected blood at that time, indicating sPLA2i-NPs were nearly cleared from circulation (FIGS. 28a,b). One month later, no fluorescence was observed in the liver and kidney.

### Example 11: sPLA2i-NPs Block Cartilage Degeneration in OA Cartilage Explants

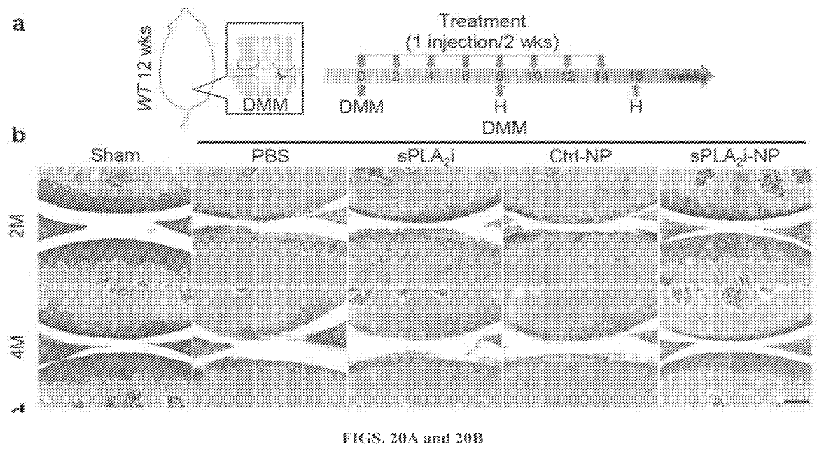

Articular cartilage explants represent an in vitro model to study OA progression in a three dimensional (3D) environment(31). We harvested femoral head cartilage explants from 2-month-old wild-type (WT) mice and then stimulated them with OA-associated pro-inflammatory cytokine interleukin-1β (IL-1β)(32). The penetration of sPLA2i-NPs was studied by acquiring fluorescence images of IL-1β-stimulated cartilage explants that were incubated with rhodamine-labeled sPLA2i-NPs for 24 or 48 hours. It was found that fluorescence was mainly present in the cartilage surface at 24 hours (FIG. 19a), while a strong fluorescence signal was observed in the deep calcified layer at 48 hours (FIGS. 19b,c), indicating that sPLA2i-NPs are able to penetrate into femoral head cartilage treated with IL-1β. These results were consistent with those presented above for sPLA2i-NPs penetration within bovine cartilage explants.

To test the therapeutic effects of sPLA2i-NPs in vitro, femoral head cartilage explants were divided into 4 groups that received PBS, IL-1β and PBS, IL-1β and Ctrl-NPs, or IL-1β and sPLA2i-NP treatments, respectively, for 8 days. As shown in FIGS. 19d-f, IL-1β treatment led to an OA-like phenotype in cartilage, featured by surface fibrillation and proteoglycan loss. Strikingly, sPLA2i-NPs, but not Ctrl-NPs, blocked cartilage degeneration induced by IL-113, leading to comparable Safranin-O content and Mankin score as the untreated group. sPLA2i-NPs, but not PBS or Ctrl-NPs, attenuated M-Iβ-induced sPLA2-IIA expression in cartilage (FIG. 19g). Furthermore, the catabolic effects of IL-1β, such as decreasing the expression of matrix proteins, Col2a1 and Aggrecan, and increasing the expression of proteinases, Mmp13 and Adamts5, were effectively reversed by sPLA2i-NPs. (FIG. 19g). The sPLA2i (thioetheramide-PC) used here is a structurally modified phospholipid that functions as a competitive, reversible inhibitor of secretory phospholipase A2 (sPLA2)(33). Inhibition itself might not be a direct regulator of the sPLA2-IIA expression; however, it likely breaks down the positive feedback loop of sPLA2 and inflammation and thus reduces the expression of sPLA2 by lowering the level of inflammation. Taken together, these data provide ex vivo evidence that sPLA2i-NPs have protective action on chondrocytes against OA inducing insults.

### Example 12: sPLA2i-NPs Attenuate Joint Destruction in a Surgery-Induced Mouse OA Model

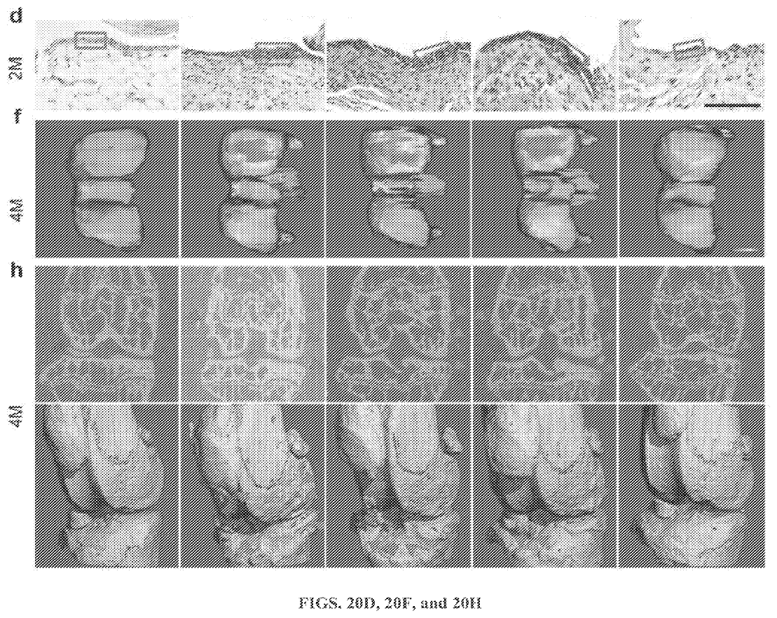

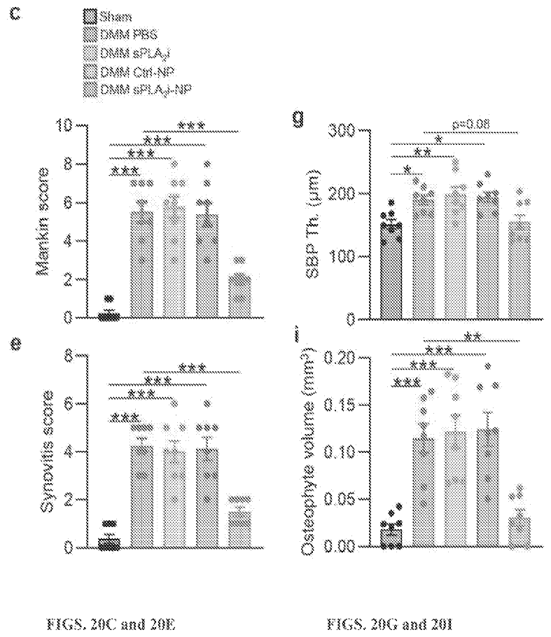

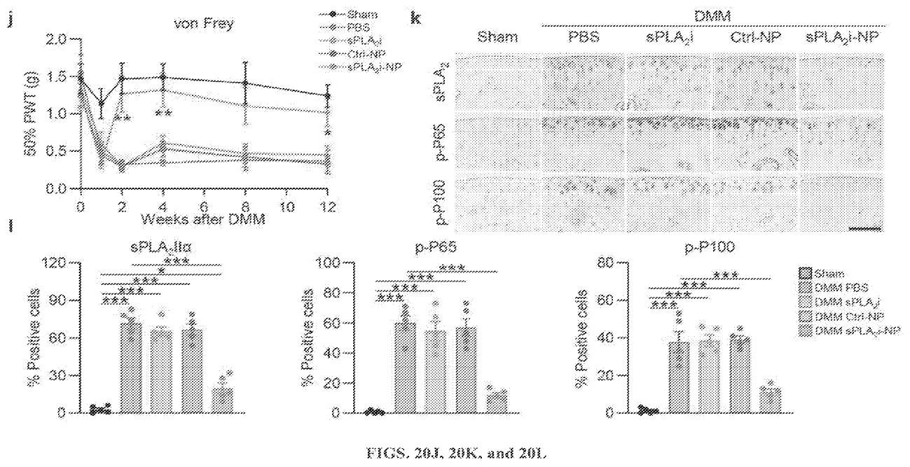

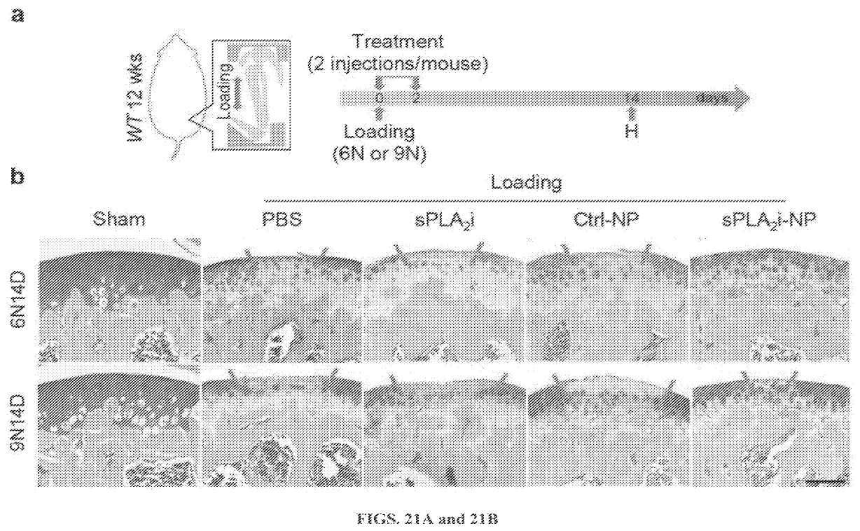

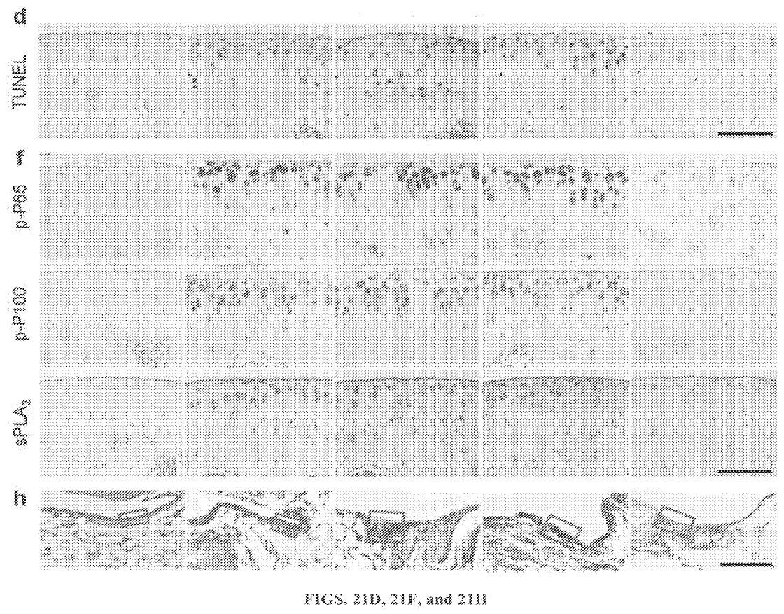

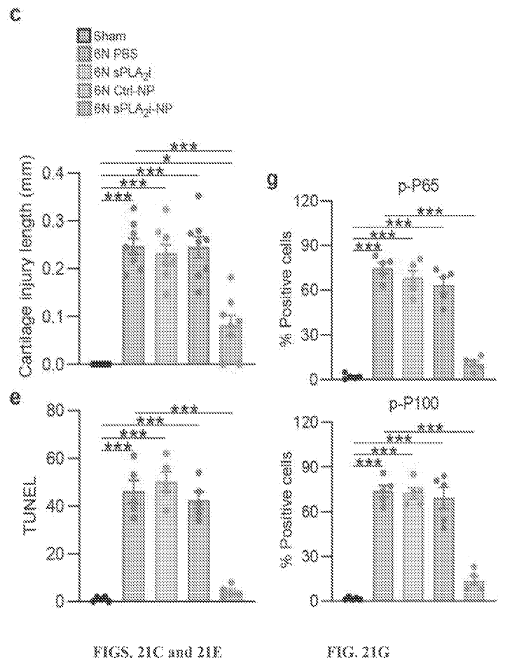

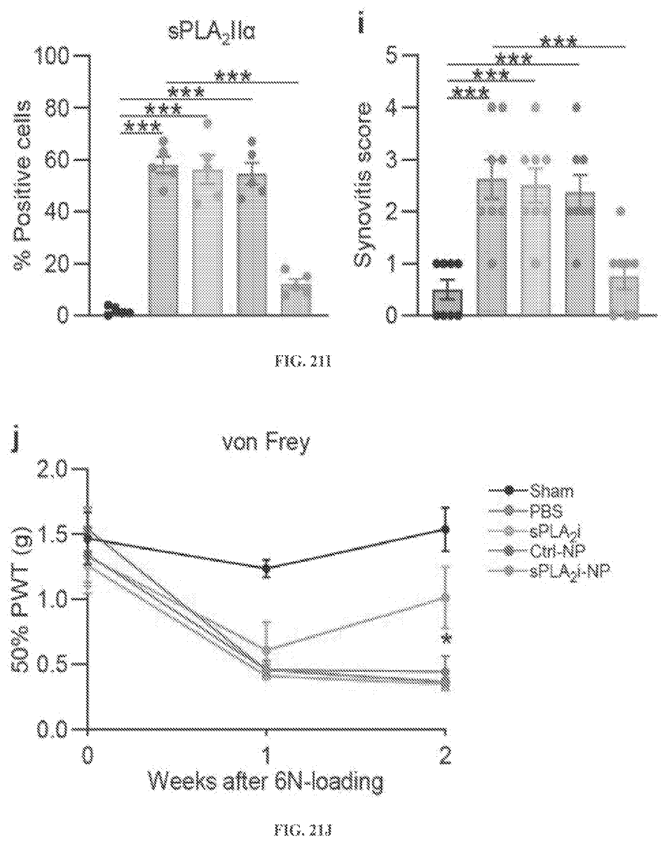

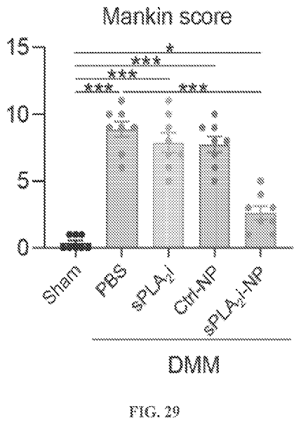

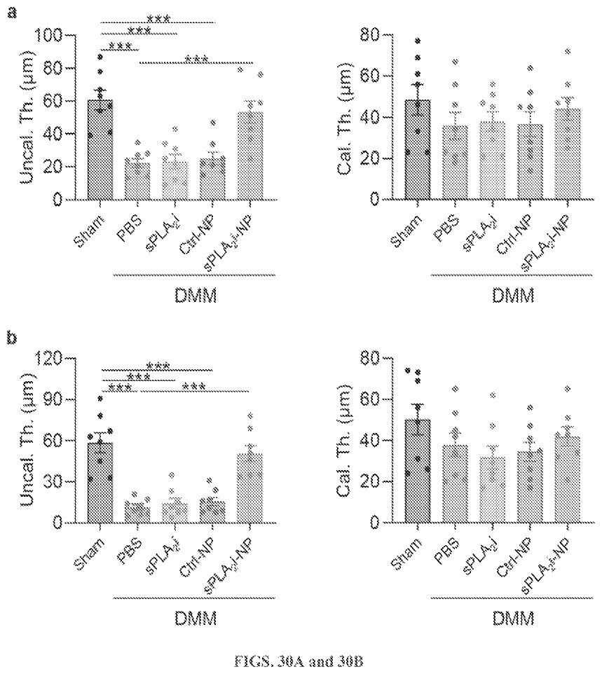

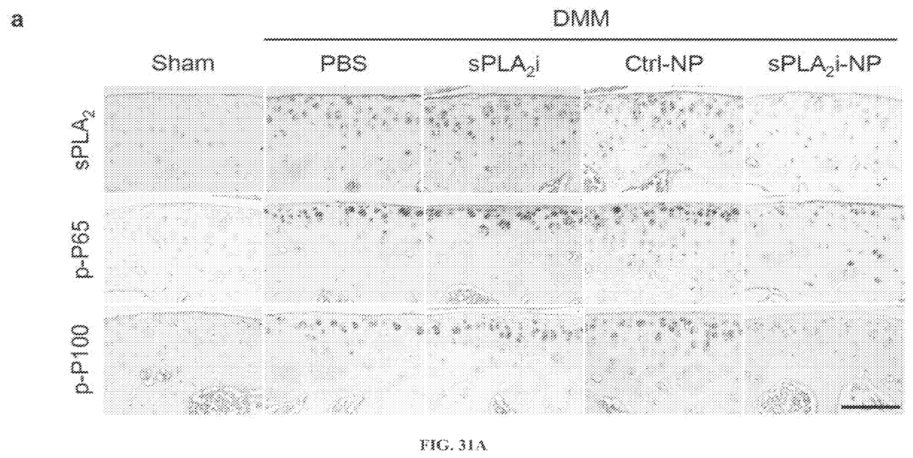

To study the in vivo therapeutic effects of sPLA2i-NPs, we used two mouse OA models. The first, a DMM surgery model, mimics chronic OA development in human patients (FIG. 20a). After DMM, WT knees started to show cartilage damage, including surface fibrillation and loss of proteoglycan, at 2 months, and exhibited severe cartilage erosion up to the entire uncalcified zone at 4 months, resulting in Mankin scores of 5.5 and 8.9, at these two time points respectively (FIGS. 20b,c and FIGS. 28 and 29). Intra-articular injection of sPLA2i-NPs into DMM knees, once every two weeks, starting immediately after the surgery, greatly improved the morphology and structure of articular cartilage, leading to an almost intact cartilage surface with no proteoglycan loss even at 4 months post-surgery (FIG. 20b and FIGS. 28 and 29b). These therapeutic effects were not observed with free sPLA2i or Ctrl-NP treatments.

The thickness of synovium is an indicator of inflammation in the knee joint.(34, 35) PBS-, sPLA2i-, and Ctrl-NP-treated DMM knees exhibited a significantly thickened synovial lining with more than 10-fold increase in the synovitis score compared with sham knees. In contrast, sPLA2i-NP-treated DMM knees had only a 4-fold increase (FIGS. 20d,e), suggesting that sPLA2i-NPs indeed generate an anti-inflammatory effect in the injured knees.

We further characterized some of the late OA symptoms including subchondral bone plate (SBP) sclerosis and osteophyte formation. At 4 months after DMM surgery, the thickness of the SBP at the femoral medial site in the knee joint was significantly increased in PBS-(1.25-fold), sPLA2i-(1.31-fold), and Ctrl-NP-(1.29-fold) treated groups compared to that in sham joints (FIGS. 20f,g). However, this SBP sclerosis was not observed in the sPLA2i-NP-treated group. Furthermore, osteophytes were also frequently found in DMM knees in control treatment groups but not in the sPLA2i-NP-treated group (FIGS. 20h,i). These data clearly indicate that sPLA2i-NPs prevent OA progression into a late stage.

In WT mice, mechanical allodynia was observed in the operated hind paw after DMM surgery but not following sham surgery.(36) The mechanical threshold in DMM knees was greatly reduced compared to sham knees. Administration of PBS, sPLA2i, or Ctrl-NPs into DMM knees did not alter the OA pain (FIG. 20j). DMM knees receiving sPLA2i-NPs initially exhibited pain response at 1 week, probably due to the persistent post-surgical pain. At 2 weeks post-surgery, sPLA2i-NP treatment greatly increased the mechanical threshold to a level close to that in sham knees and maintained that level throughout the experimental period, implying that sPLA2i-NP treatment relieves OA pain.

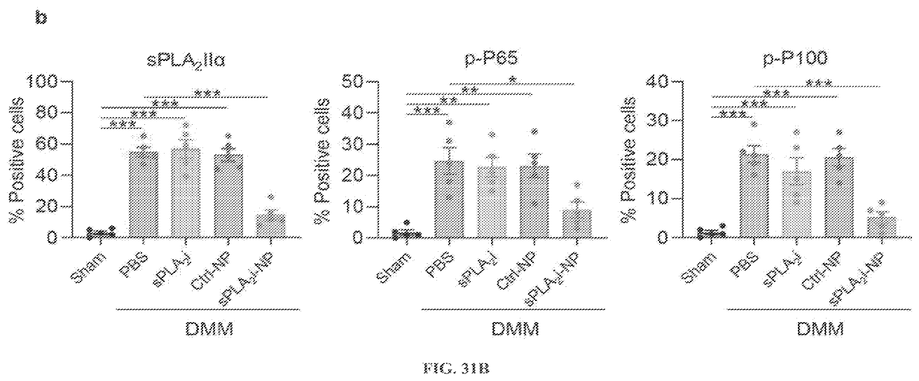

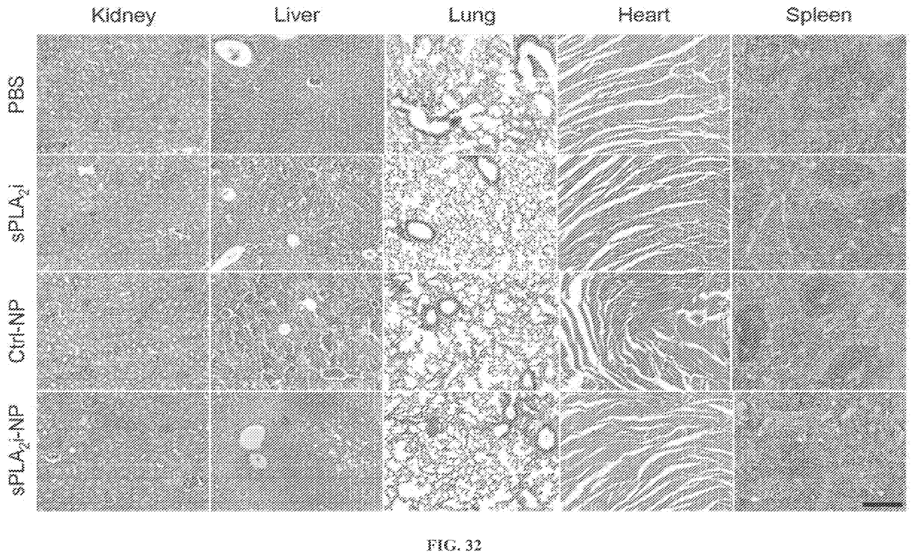

We further studied the mechanism of sPLA2 in inhibition for treating OA. IHC showed that sPLA2i-NPs blocked the up-regulation of sPLA2 after OA induction (FIGS. 20k,l). Consequently, while DMM elevated the inflammatory signaling pathway, such as phosphor (p)-p65 and p-p100, sPLA2i-NP treatment greatly decreased the amounts of these inflammatory indicators in the cartilage at 2 and 4 months after DMM, thus protecting cartilage from OA insults (FIGS. 20k,l and FIG. 31). Collectively, these results suggest that the sPLA2 enzyme mediates the pathogenesis of OA, while sPLA2i-NPs inhibit OA inflammation in DMM knee joints.

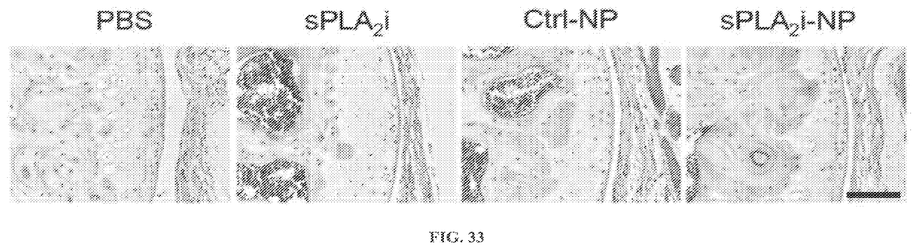

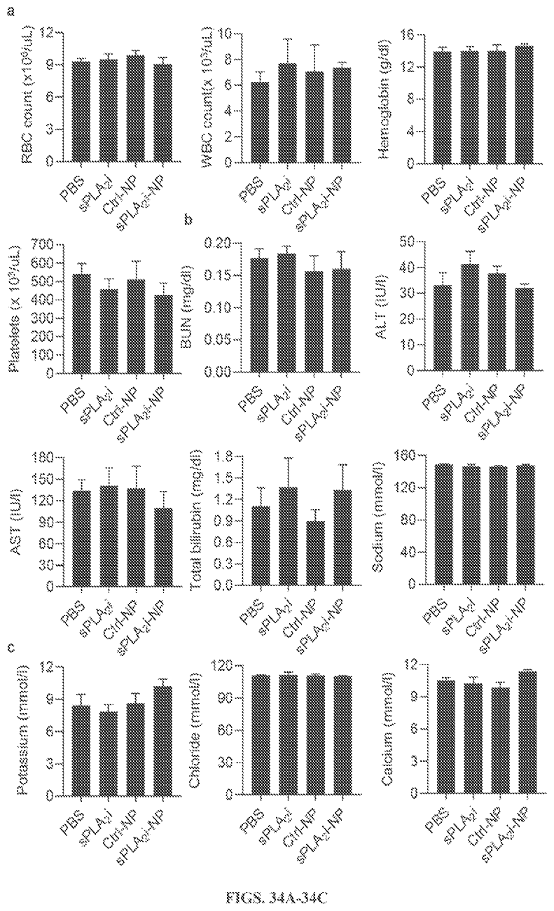

To assess the toxicity of sPLA2i-NPs in mice, the histopathology of tissues from the liver, spleen, kidney, lung, heart and knee joint of mice was evaluated two months after serial intra-articular injections of PBS, sPLA2i, Ctrl-NPs or sPLA2i-NPs. We did not observe any histopathological abnormalities in any of the mouse organs and knee joints which received these injections directly (FIGS. 32 and 33). Furthermore, we evaluated toxic effects on blood parameters of mice following treatment and did not observe any clinical signs of toxicity (FIG. 34). These results suggest that sPLA2i-NPs at the adopted dosages show no toxicity in mice.

### Example 13: sPLA2i-NPs Block Joint Damage in a Single Load-Induced Mouse PTOA Model

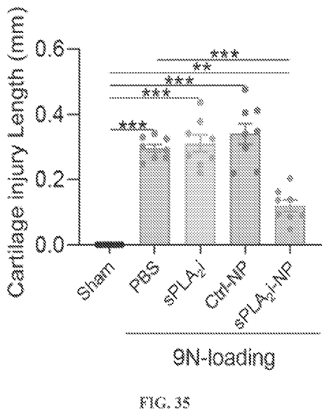

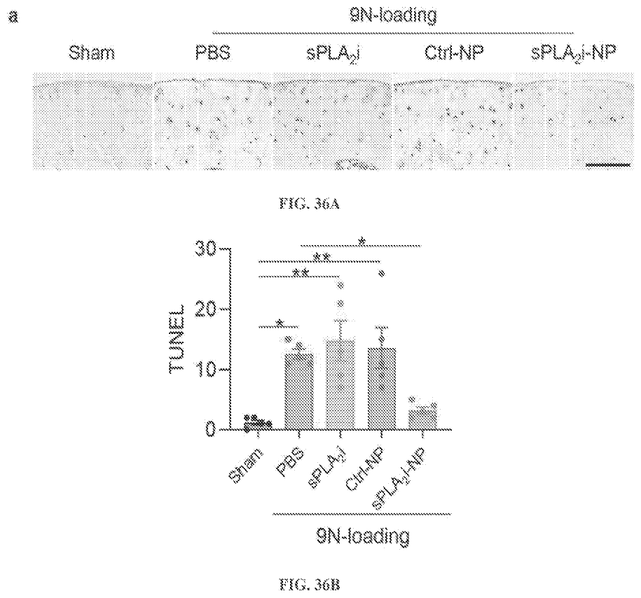

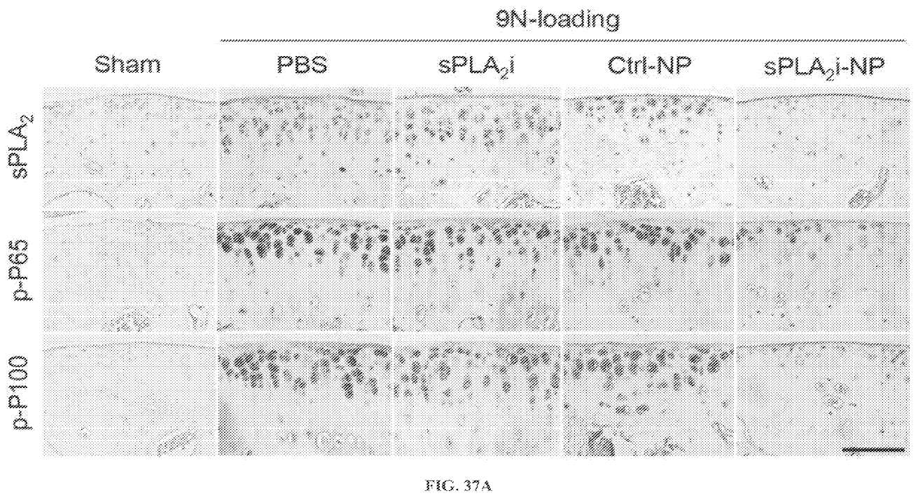

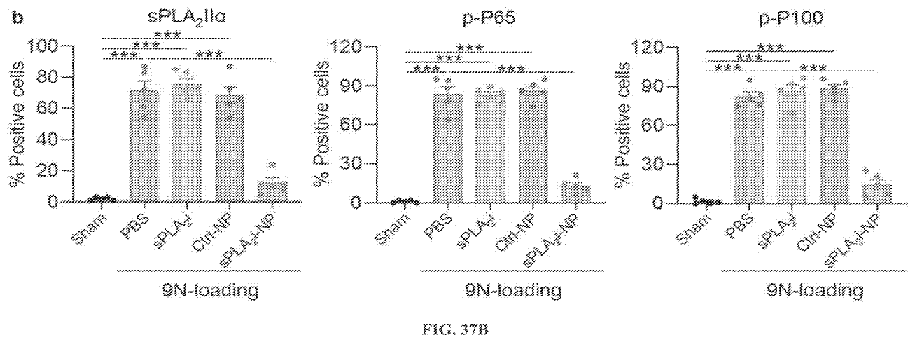

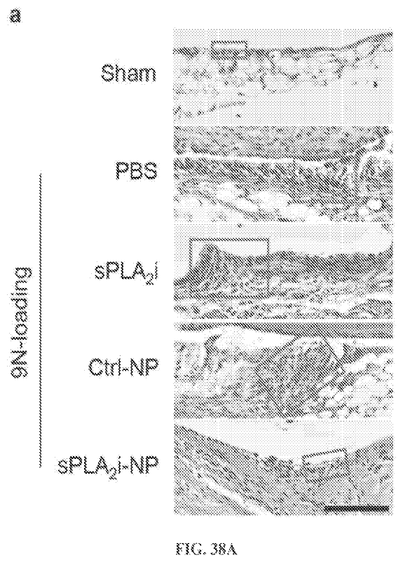

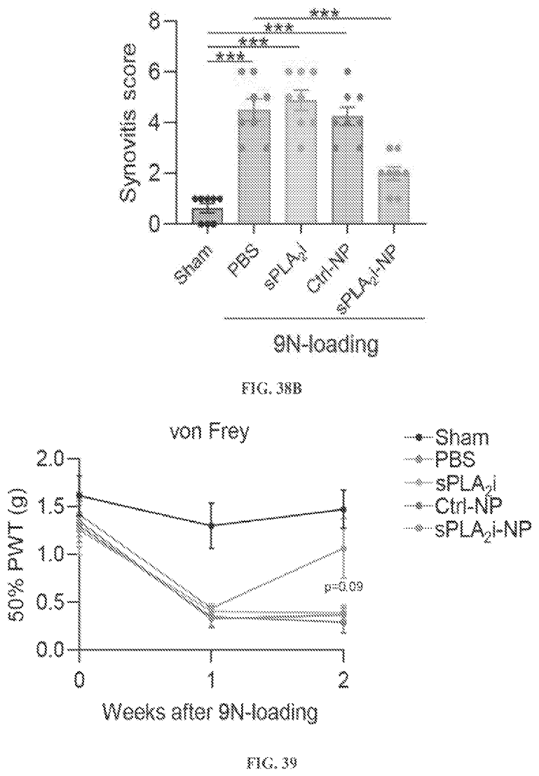

The second mouse OA model we tested is a non-invasive mechanical loading model, which is clinically relevant to post-traumatic OA (PTOA) (FIG. 21a). This injury model allows for the study of early events after impact, at a time when inflammation is thought to be particularly important(29). We applied a single loading episode, composed of 60 cycles of 6 N or 9 N peak load, on the mouse tibia to induce joint translation and anterior cruciate ligament (ACL) rupture. Two weeks later, a lesion was seen alongside with proteoglycan loss in the lateral femoral articular cartilage surface (FIG. 21b). Quantification of the length of cartilage injury revealed significant damage areas in PBS-, sPLA2i-, and Ctrl-NP-treated knees (FIG. 21c and FIG. 35). As expected, 9 N generated more severe damage than 6 N in all groups. In this model, chondrocyte apoptosis at the loading site contributes significantly to OA development. Interestingly, TUNEL staining revealed that sPLA2i-NP treatment greatly reduced the number of apoptotic chondrocytes in cartilage following loading (FIGS. 21d,e and FIG. 36). Examining the inflammatory pathway again revealed a reduction of p-P65 and p-P100 in sPLA2i-NP-treated knee joints, but not in sPLA2i or Ctrl-NP groups (FIGS. 21f,g and FIG. 37). Similar to the above DMM model, we found that sPLA2i-NPs suppressed the amount of sPLA2-IIA enzyme in cartilage. Synovitis scores suggested significant synovitis in PBS-, sPLA2i-, and Ctrl-NP-treated knees (FIGS. 21h,i and FIG. 38). A von Frey assay clearly showed that mice in these groups experienced OA pain (FIG. 21j and FIG. 39). In contrast, injections of sPLA2i-NPs into knee joints immediately and 48 hours after loading remarkably reversed these PTOA symptoms regardless of loading force. Specifically, compared to PBS-treated knees, the length of cartilage injury in sPLA2i-NP-treated knees was decreased by 67% (6N) and 59% (9 N); synovitis score was reduced by 71% (6N) and 56% (9 N); and pain threshold was increased to a level similar to sham knees. Hence, our data demonstrate a joint protective role of sPLA2i-NPs in load-induced OA.

### Example 14: Discussion

Growing evidence suggests that inflammation has a critical role in the pathogenesis of OA. In this work, we show that the level of sPLA2 is significantly increased in articular cartilage in both human and mouse OA knee tissues. These findings led to our central hypothesis that sPLA2 could act as a novel therapeutic target for OA treatment. Our strategy was to develop a sPLA2i-based approach since the release of free AA from membrane phospholipids by sPLA2 is one of the major contributors to producing potent inflammatory mediators such as eicosanoids or platelet-activating factor. Compared to classical NSAIDs, inhibiting sPLA2 enzyme activity could be a more effective anti-inflammatory approach. Currently, several promising small molecule inhibitors of sPLA2 enzyme have been developed, but conventional treatment approaches with these drugs have failed in clinical trials due to poor pharmacokinetic profiles, off-target toxicity, or inadequate efficacy. This is particularly the case for joint diseases, such as OA, because small molecules have a high rate of clearance from the joint space. Our results supported this limitation, showing that sPLA2i alone did not have a protective effect on cartilage degeneration in OA animals.

In this study, we have addressed the sPLA2i delivery challenges by incorporating small sPLA2i into nanometer-sized phospholipid nanoparticles, namely sPLA2i-NPs. This delivery platform is sPLA2-responsive, enabling sustained and controlled drug release based on the pathological conditions during OA progression, which offers a unique and novel long-term clinical option to treat patients with OA. By tuning the size and surface charge, the sPLA2i-NPs showed deep cartilage penetration and prolonged residence time in knee joints. In an ex vivo explant model in which femoral head explants were challenged with IL-113 to induce inflammation-mediated cartilage degradation, treatment with sPLA2i-NPs protected chondrocytes against IL-1β insult. We further confirmed the successful application of sPLA2i-NPs for OA treatment using two in vivo mouse models of OA. In the DMM model, local delivery of sPLA2i-NPs slowed the progression of cartilage degeneration, reduced synovial inflammation, prevented osteophyte formation, delayed the progression into a late OA stage, and relieved join pain. Similar therapeutic efficacy was also observed in a single load-induced PTOA model. These results provide strong evidence that sPLA2i-NPs can act as an effective therapeutic agent for OA treatment.

Since the main components of sPLA2i-NPs are FDA-approved biocompatible materials, it was not surprising to find that the sPLA2i-NPs did not show any signs of toxicity on the cellular, tissue, and organ levels. In addition, the method for producing sPLA2i-NPs is highly reproducible, simple and cost-effective since all components are included in starting materials prior to sPLA2i-NP formation, which would allow large-scale, GMP production of nanoparticles, a necessary step for the initiation of future clinical trials. It should be mentioned that the sPLA2i-NPs administration was initiated immediately after DMM surgery in this study. Future animal studies should incorporate injections at time points when early or middle-stage OA symptoms appear. Given that sPLA2 level was drastically elevated in human knee cartilage at early-, middle-, and late-stage of OA progression, it is reasonable to expect that the sPLA2i-NPs could also be used to slow cartilage degeneration and OA progression in cases where OA is already established. In addition, preclinical investigation in larger animals is necessary for clinical translation and regulatory approval.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Example 15. sPLA2i-NP with Varespladib (Varespladib-NPs)

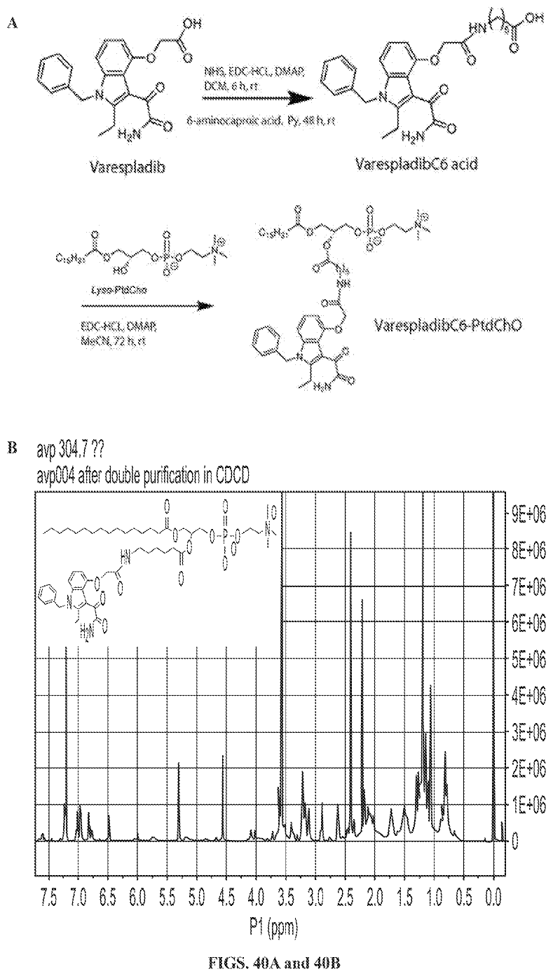

Varespladib is a clinically-tested and highly potent sPLA2 inhibitor. To load this drug into phospholipid micelles, a varespladib-lipid conjugate was synthesized by reacting varespladib with L-α-lysophosphatidylcholine (PtdCHO) (FIG. 40A). The —COOH from varespladib and —OH from lysophosphatidylcholine formed the sPLA2-cleavable sn-2 ester bond. To control the selective release of varespladib by sPLA2, aminocaproic spacers (C6-) between the varespladib and the lipid backbone was introduced using carbodiimide crosslinker chemistry. The structure of synthesized varespladib-lipid conjugate was confirmed by NMR (FIG. 40B).

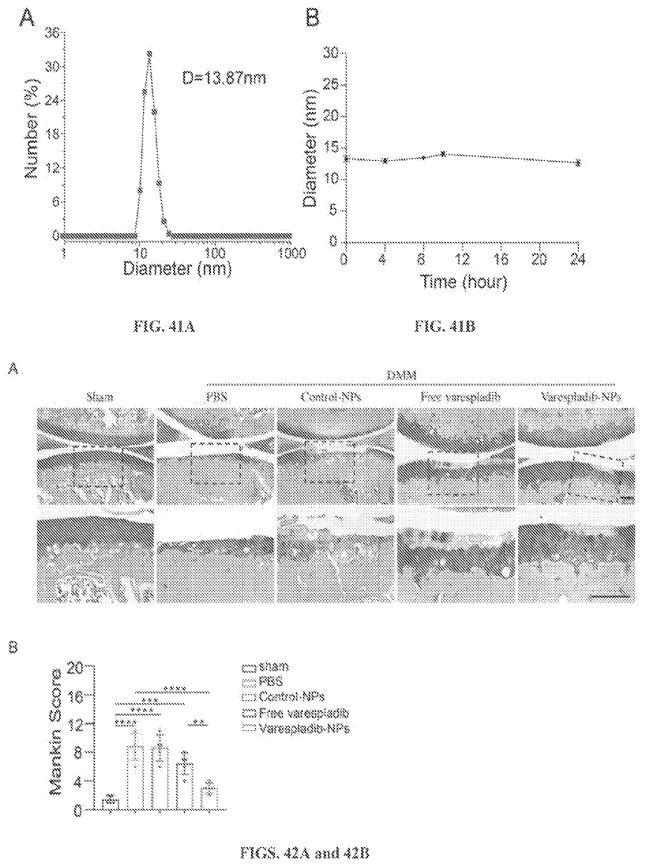

Varespladib-NPs were formed by incorporating 25 mol % varespladib-lipid conjugate into phospholipid micelles with 10 mol % DOTAP/65 mol % DSPE-PEG2000. The varespladib-NPs had a hydrodynamic diameter of 13.87 nm (FIG. 41A). There was no observable size change of varespladib-NPs in bovine synovial fluid for 24 hours (FIG. 41B).

To study the in vivo therapeutic effects of varespladib-NPs, we injected 10 μL varespladib-NPs (varespladib concentration: 60 μM) into mouse knee joints immediately after DMM surgery once every 3 weeks. Control groups include knee joints injected with 10 μL phosphate-buffered saline (PBS), Control-NP (i.e. no varespladib), and free varespladib (60 μM). The sham group without receiving any treatment is also included. Compared to control group treated with free varespladib at 3 months after DMM surgery, the morphology and structure of the articular cartilage from group treated with varespladib-NPs was significantly improved (FIGS. 42A and 41B). These therapeutic effects were not observed with PBS or Ctrl-NP treatments.

### REFERENCES FOR EXAMPLES 1-6

1 Moalem, G. & Tracey, D. J. Immune and inflammatory mechanisms in neuropathic pain. Brain Res Rev 51, 240-264, doi:10.1016/j.brainresrev.2005.11.004 (2006).
2 Harden, N. & Cohen, M. Unmet needs in the management of neuropathic pain. J Pain Symptom Manage 25, S12-17 (2003).
3 Scholz, J. & Woolf, C. J. The neuropathic pain triad: neurons, immune cells and glia. Nat Neurosci 10, 1361-1368, doi:10.1038/nn1992 (2007).
4 Radhakrishnan, K., Litchy, W. J., O'Fallon, W. M. & Kurland, L. T. Epidemiology of cervical radiculopathy. A population-based study from Rochester, Minn., 1976 through 1990. Brain 117 (Pt 2), 325-335 (1994).
5 Waters, T. R. National efforts to identify research issues related to prevention of work-related musculoskeletal disorders. J Electromyogr Kinesiol 14, 7-12 (2004).
6 Rempel, D. M., Harrison, R. J. & Barnhart, S. Work-related cumulative trauma disorders of the upper extremity. Jama 267, 838-842 (1992).
7 Cote, P., Cassidy, J. D., Carroll, L. J. & Kristman, V. The annual incidence and course of neck pain in the general population: a population-based cohort study. Pain 112, 267-273, doi:S0304-3959(04)00426-9 [pii]10.1016/j.pain.2004.09.004 (2004).
8 Cote, P., Cassidy, J. D. & Carroll, L. The Saskatchewan Health and Back Pain Survey. The prevalence of neck pain and related disability in Saskatchewan adults. Spine 23, 1689-1698 (1998).
9 Lin, Y.-W., Cheng, C.-M., LeDuc, P. R. & Chen, C.-C. Understanding Sensory Nerve Mechanotransduction through Localized Elastomeric Matrix Control. PLoS ONE 4, e4293 (2009).
10 Lehto, I. J. et al. Age-related MRI changes at 0.1 T in cervical discs in asymptomatic subjects. Neuroradiology 36, 49-53 (1994).
11 Matsumoto, M. et al. MRI of cervical intervertebral discs in asymptomatic subjects. J Bone Joint Surg Br 80, 19-24 (1998).
12 Schellhas, K. P., Smith, M. D., Gundry, C. R. & Pollei, S. R. Cervical discogenic pain. Prospective correlation of magnetic resonance imaging and discography in asymptomatic subjects and pain sufferers. Spine 21, 300-311; discussion 311-302 (1996).
13 Carette, S. & Fehlings, M. G. Clinical practice. Cervical radiculopathy. N Engl J Med 353, 392-399, doi:353/4/392 [pii]10.1056/NEJMcp043887 (2005).
14 Harden, R. N. Chronic neuropathic pain. Mechanisms, diagnosis, and treatment. Neurologist 11, 111-122, doi:10.1097/01.nr1.0000155180.60057.8e (2005).
15 Gilron, I., Baron, R. & Jensen, T. Neuropathic pain: principles of diagnosis and treatment. Mayo Clin Proc 90, 532-545, doi: 10.1016/j.mayocp.2015.01.018 (2015).
16 Henschke, N., Kamper, S. J. & Maher, C. G. The Epidemiology and Economic Consequences of Pain. Mayo Clinic Proceedings 90, 139-147, doi:10.1016/j.mayocp.2014.09.010 (2015).
17 Chizh, B. A. & Headley, P. M. NMDA antagonists and neuropathic pain—Multiple drug targets and multiple uses. Curr Pharm Design 11, 2977-2994, doi:Doi 10.2174/1381612054865082 (2005).
18 Watkins, L. R., Milligan, E. D. & Maier, S. F. Spinal cord glia: new players in pain. Pain 93, 201-205, doi:Doi 10.1016/S0304-3959(01)00359-1 (2001).
19 Rutkowski, M. D., Winkelstein, B. A., Hickey, W. F., Pahl, J. L. & DeLeo, J. A. Lumbar nerve root injury induces central nervous system neuroimmune activation and neuroinflammation in the rat: relationship to painful radiculopathy. Spine 27, 1604-1613, doi:00007632-200208010-00003 [pii] (2002).
20 Trbovich, M. & Yang, H. Capsaicin 8% Patch for Central and Peripheral Neuropathic Pain of Persons with Incomplete Spinal Cord Injury: Two Case Reports. Am J Phys Med Rehabil 94, e66-72, doi:10.1097/PHM.0000000000000301 (2015).
21 Hubbard, R. D., Chen, Z. & Winkelstein, B. A. Transient cervical nerve root compression modulates pain: load thresholds for allodynia and sustained changes in spinal neuropeptide expression. J Biomech 41, 677-685, doi:S0021-9290(07)00402-2 [pii] 10.1016/j.jbiomech.2007.09.026 (2008).
22 Rothman, S. M. & Winkelstein, B. A. Chemical and mechanical nerve root insults induce differential behavioral sensitivity and glial activation that are enhanced in combination. Brain Res 1181, 30-43, doi:S0006-8993(07)01993-2 [pii] 10.1016/j.brainres.2007.08.064 (2007).
23 Kelly, J. D. t., Aliquo, D., Sitler, M. R., Odgers, C. & Moyer, R. A. Association of burners with cervical canal and foraminal stenosis. Am J Sports Med 28, 214-217 (2000).
24 Nuckley, D. J., Konodi, M. A., Raynak, G. C., Ching, R. P. & Mirza, S. K. Neural space integrity of the lower cervical spine: effect of normal range of motion. Spine 27, 587-595 (2002).
25 Thorek, D. L., Weisshaar, C. L., Czupryna, J. C., Winkelstein, B. A. & Tsourkas, A. Superparamagnetic iron oxide-enhanced magnetic resonance imaging of neuroinflammation in a rat model of radicular pain. Mol Imaging 10, 206-214 (2011).
26 Znaor, L., Lovric, S., Hogan, Q. & Sapunar, D. Association of neural inflammation with hyperalgesia following spinal nerve ligation. Croat Med J 48, 35-42 (2007).
27 Hashizume, H., DeLeo, J. A., Colburn, R. W. & Weinstein, J. N. Spinal glial activation and cytokine expression after lumbar root injury in the rat. Spine (Phila Pa. 1976) 25, 1206-1217 (2000).
28 Winkelstein, B. A. & DeLeo, J. A. Nerve root injury severity differentially modulates spinal glial activation in a rat lumbar radiculopathy model: considerations for persistent pain. Brain Res 956, 294-301 (2002).
29 DeLeo, J. A. & Yezierski, R. P. The role of neuroinflammation and neuroimmune activation in persistent pain. Pain 90, 1-6 (2001).
30 Sweitzer, S. M., Hickey, W. F., Rutkowski, M. D., Pahl, J. L. & DeLeo, J. A. Focal peripheral nerve injury induces leukocyte trafficking into the central nervous system: potential relationship to neuropathic pain. Pain 100, 163-170, doi:Pii S0304-3959(02)00257-9 Doi 10.1016/S0304-3959(02)00257-9 (2002).
31 Rutkowski, M. D., Pahl, J. L., Sweitzer, S., van Rooijen, N. & DeLeo, J. A. Limited role of macrophages in generation of nerve injury-induced mechanical allodynia. Physiol Behav 71, 225-235, doi:S0031-9384(00)00333-4 [pii] (2000).
32 Sweitzer, S., Martin, D. & DeLeo, J. A. Intrathecal interleukin-1 receptor antagonist in combination with soluble tumor necrosis factor receptor exhibits an anti-allodynic action in a rat model of neuropathic pain. Neuroscience 103, 529-539, doi:S0306-4522(00)00574-1 [pii] (2001).
33 Watkins, L. R. et al. Characterization of cytokine-induced hyperalgesia. Brain Res 654, 15-26, doi:0006-8993(94)91566-0 [pii] (1994).
34 Winkelstein, B. A., Rutkowski, M. D., Sweitzer, S. M., Pahl, J. L. & DeLeo, J. A. Nerve injury proximal or distal to the DRG induces similar spinal glial activation and selective cytokine expression but differential behavioral responses to pharmacologic treatment. J Comp Neurol 439, 127-139, doi:10.1002/cne.1337 [pii] (2001).
35 McNicol, E. D., Midbari, A. & Eisenberg, E. Opioids for neuropathic pain. The Cochrane database of systematic reviews 8, Cd006146, doi:10.1002/14651858.CD006146.pub2 (2013).
36 Moore, R. A., Chi, C. C., Wiffen, P. J., Derry, S. & Rice, A. S. Oral nonsteroidal anti-inflammatory drugs for neuropathic pain. The Cochrane database of systematic reviews 10, Cd010902, doi:10.1002/14651858.CD010902.pub2 (2015).
37 Dworkin, R. H. et al. Recommendations for the pharmacological management of neuropathic pain: an overview and literature update. Mayo Clin Proc 85, S3-14, doi:10.4065/mcp.2009.0649 (2010).
38 Osikowicz, M., Mika, J. & Przewlocka, B. The glutamatergic system as a target for neuropathic pain relief. Exp Physiol 98, 372-384, doi:10.1113/expphysio1.2012.069922 (2013).
39 Wilczewska, A. Z., Niemirowicz, K., Markiewicz, K. H. & Car, H. Nanoparticles as drug delivery systems. Pharmacol Rep 64, 1020-1037, doi:Doi 10.1016/S1734-1140(12)70901-5 (2012).
40 Zhao, Z., Liu, N., Huang, J., Lu, P. H. & Xu, X. M. Inhibition of cPLA₂ activation by Ginkgo biloba extract protects spinal cord neurons from glutamate excitotoxicity and oxidative stress-induced cell death. J Neurochem 116, 1057-1065, doi:10.1111/j.1471-4159.2010.07160.x (2011).
41 Toborek, M. et al. Arachidonic acid-induced oxidative injury to cultured spinal cord neurons. J Neurochem 73, 684-692 (1999).
42 Miyamoto, H., Saura, R., Harada, T., Doita, M. & Mizuno, K. The role of cyclooxygenase-2 and inflammatory cytokines in pain induction of herniated lumbar intervertebral disc. Kobe J Med Sci 46, 13-28 (2000).
43 Carabaza, A. et al. Inhibition of phospholipase A2 purified from human herniated disc. Biochem Pharmacol 45, 783-786 (1993).
44 Biyani, A. & Andersson, G. B. Low back pain: pathophysiology and management. The Journal of the American Academy of Orthopaedic Surgeons 12, 106-115 (2004).
45 Franson, R. C., Saal, J. S. & Saal, J. A. Human disc phospholipase A2 is inflammatory. Spine (Phila Pa. 1976) 17, S129-132 (1992).
46 Gronblad, M. et al. A controlled biochemical and immunohistochemical study of human synovial-type (group II) phospholipase A2 and inflammatory cells in macroscopically normal, degenerated, and herniated human lumbar disc tissues. Spine (Phila Pa. 1976) 21, 2531-2538 (1996).
47 Korovessis, P. G. Phospholipase A2 activity in herniated lumbar discs. Spine (Phila Pa. 1976) 24, 99 (1999).
48 Piperno, M. et al. Phospholipase A2 activity in herniated lumbar discs. Clinical correlations and inhibition by piroxicam. Spine (Phila Pa. 1976) 22, 2061-2065 (1997).
49 Kawakami, M., Tamaki, T., Hayashi, N., Hashizume, H. & Nishi, H. Possible mechanism of painful radiculopathy in lumbar disc herniation. Clinical orthopaedics and related research, 241-251 (1998).
50 Kishimoto, K., Matsumura, K., Kataoka, Y., Morii, H. & Watanabe, Y. Localization of cytosolic phospholipase A2 messenger RNA mainly in neurons in the rat brain. Neuroscience 92, 1061-1077 (1999).
51 Ong, W. Y., Horrocks, L. A. & Farooqui, A. A. Immunocytochemical localization of cPLA2 in rat and monkey spinal cord. Journal of molecular neuroscience: MN 12, 123-130, doi:10.1007/bf02736926 (1999).
52 Murakami, M., Nakatani, Y., Atsumi, G., Inoue, K. & Kudo, I. Regulatory functions of phospholipase A2. Critical reviews in immunology 17, 225-283 (1997).
53 van Rossum, G. S., Drummen, G. P., Verkleij, A. J., Post, J. A. & Boonstra, J. Activation of cytosolic phospholipase A2 in Her14 fibroblasts by hydrogen peroxide: a p42/44(MAPK)-dependent and phosphorylation-independent mechanism. Biochimica et biophysica acta 1636, 183-195, doi:10.1016/j.bbalip.2003.12.008 (2004).
54 Klein, J. Membrane breakdown in acute and chronic neurodegeneration: focus on choline-containing phospholipids. Journal of neural transmission (Vienna, Austria: 1996) 107, 1027-1063 (2000).
55 Sun, G. Y., Xu, J. F., Jensen, M. D. & Simonyi, A. Phospholipase A(2) in the central nervous system: implications for neurodegenerative diseases. J Lipid Res 45, 205-213, doi:10.1194/jlr.R300016-ThR200 (2004).
56 Tang, Z. Y. et al. Changes of Cervical Dorsal Root Ganglia Induced by Compression Injury and Decompression Procedure: A Novel Rat Model of Cervical Radiculoneuropathy. J Neurotraum 26, 289-295, doi:10.1089/neu.2008.0506 (2009).
57 Gao, Q. et al. PLA₂-responsive and SPIO-loaded phospholipid micelles. Chem Commun (Camb) 51, 12313-12315, doi:10.1039/c5cc04540a (2015).
58 Cheng, Z. L., Thorek, D. L. J. & Tsourkas, A. Porous Polymersomes with Encapsulated Gd-Labeled Dendrimers as Highly Efficient MRI Contrast Agents. Adv Funct Mater 19, 3753-3759, doi:10.1002/adfm.200901253 (2009).
59 Chan, F. K., Moriwaki, K. & De Rosa, M. J. Detection of necrosis by release of lactate dehydrogenase activity. Methods Mol Biol 979, 65-70, doi:10.1007/978-1-62703-290-2_7 (2013).
60 Prabhu, B. M., Ali, S. F., Murdock, R. C., Hussain, S. M. & Srivatsan, M. Copper nanoparticles exert size and concentration dependent toxicity on somatosensory neurons of rat. Nanotoxicology 4, 150-160, doi:10.3109/17435390903337693 (2010).
61 Smith, J. R. et al. Salmon-derived thrombin inhibits development of chronic pain through an endothelial barrier protective mechanism dependent on APC. Biomaterials 80, 96-105, doi:10.1016/j.biomaterials.2015.11.062 (2016).
62 Chaplan, S. R., Bach, F. W., Pogrel, J. W., Chung, J. M. & Yaksh, T. L. Quantitative assessment of tactile allodynia in the rat paw. J Neurosci Methods 53, 55-63 (1994).
63 Nicholson, K. J., Guarino, B. B. & Winkelstein, B. A. Transient nerve root compression load and duration differentially mediate behavioral sensitivity and associated spinal astrocyte activation and mGLuR5 expression. Neuroscience 209, 187-195, doi: 10.1016/j.neuroscience.2012.02.027 (2012).
64 Zeeman, M. E., Kartha, S. & Winkelstein, B. A. Whole-Body Vibration Induces Pain and Lumbar Spinal Inflammation Responses in the Rat That Vary With the Vibration Profile. J Orthop Res 34, 1439-1446, doi:10.1002/jor.23243 (2016).

### REFERENCES FOR EXAMPLES 7-14

1. B. C. Geiger, S. Wang, R. F. Padera, A. J. Grodzinsky, P. T. Hammond, Cartilage-penetrating nanocarriers improve delivery and efficacy of growth factor treatment of osteoarthritis. Sci Transl Med 10, 1-12 (2018).
2. L. Kou et al., Biomaterial-engineered intra-articular drug delivery systems for osteoarthritis therapy. Drug Deliv 26, 870-885 (2019).
3. M. A. Karsdal et al., Disease-modifying treatments for osteoarthritis (DMOADs) of the knee and hip: lessons learned from failures and opportunities for the future. Osteoarthritis Cartilage 24, 2013-2021 (2016).
4. J. Sokolove, C. M. Lepus, Role of inflammation in the pathogenesis of osteoarthritis: latest findings and interpretations. Ther Adv Musculoskelet Dis 5, 77-94 (2013).
5. J. Martel-Pelletier, Pathophysiology of osteoarthritis. Osteoarthritis Cartilage 12 Suppl A, S31-33 (2004).
6. J. B. Lin, S. Poh, A. Panitch, Controlled release of anti-inflammatory peptides from reducible thermosensitive nanoparticles suppresses cartilage inflammation. Nanomedicine 12, 2095-2100 (2016).
7. H. N. Daghestani, C. F. Pieper, V. B. Kraus, Soluble macrophage biomarkers indicate inflammatory phenotypes in patients with knee osteoarthritis. Arthritis Rheumatol 67, 956-965 (2015).
8. K. E. Galluzzi, Management of neuropathic pain. J Am Osteopath Assoc 105, S12-19 (2005).
9. J. E. Burke, E. A. Dennis, Phospholipase A2 structure/function, mechanism, and signaling. J Lipid Res 50 Suppl, S237-242 (2009).
10. Z. Zhao, N. Liu, J. Huang, P. H. Lu, X. M. Xu, Inhibition of cPLA₂ activation by Ginkgo biloba extract protects spinal cord neurons from glutamate excitotoxicity and oxidative stress-induced cell death. J Neurochem 116, 1057-1065 (2011).
11. M. Toborek et al., Arachidonic acid-induced oxidative injury to cultured spinal cord neurons. J Neurochem 73, 684-692 (1999).
12. A. Carabaza et al., Inhibition of phospholipase A2 purified from human herniated disc. Biochem Pharmacol 45, 783-786 (1993).
13. A. Biyani, G. B. Andersson, Low back pain: pathophysiology and management. The Journal of the American Academy of Orthopaedic Surgeons 12, 106-115 (2004).
14. R. C. Franson, J. S. Saal, J. A. Saal, Human disc phospholipase A2 is inflammatory. Spine (Phila Pa. 1976) 17, S129-132 (1992).
15. P. G. Korovessis, Phospholipase A2 activity in herniated lumbar discs. Spine (Phila Pa. 1976) 24, 99 (1999).
16. M. Kawakami, T. Tamaki, N. Hayashi, H. Hashizume, H. Nishi, Possible mechanism of painful radiculopathy in lumbar disc herniation. Clinical orthopaedics and related research, 241-251 (1998).
17. M. Murakami, Y. Nakatani, G. Atsumi, K. Inoue, I. Kudo, Regulatory functions of phospholipase A2. Critical reviews in immunology 17, 225-283 (1997).
18. L. Leistad, A. J. Feuerherm, A. Faxvaag, B. Johansen, Multiple phospholipase A2 enzymes participate in the inflammatory process in osteoarthritic cartilage. Scand J Rheumatol 40, 308-316 (2011).
19. G. S. van Rossum, G. P. Drummen, A. J. Verkleij, J. A. Post, J. Boonstra, Activation of cytosolic phospholipase A2 in Her14 fibroblasts by hydrogen peroxide: a p42/44(MAPK)-dependent and phosphorylation-independent mechanism. Biochimica et biophysica acta 1636, 183-195 (2004).
20. O. S. Jamal et al., Increased expression of human type IIa secretory phospholipase A2 antigen in arthritic synovium. Ann Rheum Dis 57, 550-558 (1998).
21. W. Pruzanski, E. Bogoch, M. Wloch, P. Vadas, The role of phospholipase A2 in the physiopathology of osteoarthritis. J Rheumatol Suppl 27, 117-119 (1991).
22. W. Pruzanski, E. Bogoch, E. Stefanski, M. Wloch, P. Vadas, Enzymatic activity and distribution of phospholipase A2 in human cartilage. Life Sci 48, 2457-2462 (1991).
23. W. Zhang et al., Lysophosphatidylcholines to phosphatidylcholines ratio predicts advanced knee osteoarthritis. Rheumatology (Oxford) 55, 1566-1574 (2016).
24. N. D. Quach, R. D. Arnold, B. S. Cummings, Secretory phospholipase A2 enzymes as pharmacological targets for treatment of disease. Biochem Pharmacol 90, 338-348 (2014).
25. C. D. DiDomenico, M. Lintz, L. J. Bonassar, Molecular transport in articular cartilage—what have we learned from the past 50 years? Nat Rev Rheumatol 14, 393-403 (2018).
26. C. H. Evans, V. B. Kraus, L. A. Setton, Progress in intra-articular therapy. Nat Rev Rheumatol 10, 11-22 (2014).
13
27. S. Kumar, I. M. Adjei, S. B. Brown, O. Liseth, B. Sharma, Manganese dioxide nanoparticles protect cartilage from inflammation-induced oxidative stress. Biomaterials 224, 119467 (2019).
28. J. McHugh, Promising drug delivery system. Nat Rev Rheumatol 15, 64 (2019).
29. H. Yan et al., Suppression of NF-kappaB activity via nanoparticle-based siRNA delivery alters early cartilage responses to injury. Proc Natl Acad Sci USA 113, E6199-E6208 (2016).
30. Q. Gao et al., PLA₂-responsive and SPIO-loaded phospholipid micelles. Chem Commun (Camb) 51, 12313-12315 (2015).
31. H. Jia et al., EGFR signaling is critical for maintaining the superficial layer of articular cartilage and preventing osteoarthritis initiation. Proc Natl Acad Sci USA 113, 14360-14365 (2016).
32. M. Kapoor, J. Martel-Pelletier, D. Lajeunesse, J. P. Pelletier, H. Fahmi, Role of proinflammatory cytokines in the pathophysiology of osteoarthritis. Nat Rev Rheumatol 7, 33-42 (2011).
33. K. Sonoki et al., Secretory PLA2 inhibitor indoxam suppresses LDL modification and associated inflammatory responses in TNFalpha-stimulated human endothelial cells. Br J Pharmacol 153, 1399-1408 (2008).
34. C. R. Scanzello, S. R. Goldring, The role of synovitis in osteoarthritis pathogenesis. Bone 51, 249-257 (2012).
35. C. Y. Wenham, P. G. Conaghan, The role of synovitis in osteoarthritis. Ther Adv Musculoskelet Dis 2, 349-359 (2010).
36. A. M. Malfait et al., ADAMTS-5 deficient mice do not develop mechanical allodynia associated with osteoarthritis following medial meniscal destabilization. Osteoarthritis Cartilage 18, 572-580 (2010).
37. T. Aigner et al., Histopathology atlas of animal model systems—overview of guiding principles. Osteoarthritis Cartilage 18 Suppl 3, S2-6 (2010).
38. M. J. Piel, J. S. Kroin, H. J. Im, Assessment of knee joint pain in experimental rodent models of osteoarthritis. Methods Mol Biol 1226, 175-181 (2015).
39. H. Jia et al., Loading-Induced Reduction in Sclerostin as a Mechanism of Subchondral Bone Plate Sclerosis in Mouse Knee Joints During Late-Stage Osteoarthritis. Arthritis Rheumatol 70, 230-241 (2018).

1. A composition comprising: a drug and a drug delivery system comprising a phospholipid that is hydrolyzed by phospholipase A2 (PLA2) enzyme.

2. The composition of claim 1, wherein the phospholipid, when hydrolyzed, allows release of the drug from the drug delivery system.

3. The composition of claim 1, wherein the phospholipid represents at least 10% by weight of the drug delivery system.

4. The composition of claim 1, wherein the drug delivery system comprises a liposome or a micelle, optionally, wherein the liposome or the micelle is labeled with an antibody, peptide, protein, aptamer, or small molecule that confers specificity for a specific target.

5. The composition of claim 4, wherein the micelle or liposome includes a nanoparticle, which consists of at least one selected from the group consisting of: superparamagnetic iron oxide (SPIO), iron oxide, an iron derivative, a magnetic nanoparticle, and gold nanoparticle, optionally, wherein the liposome or micelle includes a tracing agent comprising a radiolabel or a fluorescent dye.

6. (canceled)

7. The composition of claim 1, wherein the drug comprises at least one of a PLA2 inhibitor, an anti-inflammatory agent, or a neuromodulatory agent.

8. The composition of claim 1, wherein the drug comprises an antibody, a small-molecule inhibitor, a peptide inhibitor, or an siRNA, optionally, wherein the drug is a PLA2 inhibitor, a matrix metalloproteinase (MMP) inhibitor, or a Disintegrin-like and Metalloproteinase domain with Thrombospondin-1 repeats (ADAMTS) inhibitor.

9.-12. (canceled)

13. A method for treating a subject in need thereof, the method comprising: administering to the subject a drug delivery system comprising (i) a phospholipid that is hydrolyzed by phospholipase A2 (PLA2) enzyme and (ii) a drug.

14. The method of claim 13, wherein the phospholipid, when hydrolyzed, allows release of the drug from the drug delivery system.

15. The method of claim 13, wherein the phospholipid represents at least 5% by weight of the drug delivery system.

16. The method of claim 13, wherein the drug delivery system comprises a liposome or a micelle, optionally, wherein the liposome or the micelle is labeled with an antibody, peptide, protein, aptamer, or small molecule that confers specificity for a specific target.

17. The method of claim 16, wherein the micelle or liposome includes a nanoparticle, which consists of at least one selected from the group consisting of: superparamagnetic iron oxide (SPIO), iron oxide, an iron derivative, a magnetic nanoparticle, and gold nanoparticle, optionally, wherein the micelle or liposome includes a tracing agent comprising a radiolabel or a fluorescent dye.

18. (canceled)

19. The method of claim 13, wherein the drug comprises at least one of a PLA2 inhibitor, an anti-inflammatory agent, or a neuromodulatory agent.

20. The method of claim 19, wherein the drug comprises an antibody, a small-molecule inhibitor, a peptide inhibitor, or an siRNA, optionally, wherein the drug is a PLA2 inhibitor, a matrix metalloproteinase (MMP) inhibitor, or a Disintegrin-like and Metalloproteinase domain with Thrombospondin-1 repeats (ADAMTS) inhibitor.

21. (canceled)

22. The method of claim 13, wherein the subject is suffering from an injury, radiculopathy, neuropathy, cancer, shingles, a neuropathic pain, osteoarthritis (OA), or disease or a condition associated with inflammation.

23.-25. (canceled)

26. The method of claim 13, further comprising determining a location, within the subject, of the drug delivery system, optionally, wherein the determining comprises magnetic resonance imaging, fluorescent imaging, computed tomography, nuclear imaging, or ultrasound.

27. (canceled)

28. A method for determining the location of a region to be treated or monitored in a subject in need thereof, the method comprising:
administering to the subject a composition comprising a phospholipid that is hydrolyzed by phospholipase A2 (PLA2) enzyme, optionally comprising a drug; and
determining a location, within the subject, of any component of the composition,
wherein the determining comprises magnetic resonance imaging, fluorescent imaging, computed tomography, nuclear imaging, or ultrasound.

29. The method of claim 28, the composition comprises a liposome or a micelle, optionally, wherein the micelle or the liposome comprises a nanoparticle, which consists of at least one selected from the group consisting of: superparamagnetic iron oxide (SPIO), iron oxide, an iron derivative, a magnetic nanoparticle and gold nanoparticle, and/or wherein the liposome or micelle includes a tracing agent comprising a radiolabel or a fluorescent dye.

30.-34. (canceled)

35. The method of claim 13, wherein the subject is a human.

36.-41. (canceled)

42. The composition of claim 1, wherein the drug in the drug delivery system is conjugated to the phospholipid, optionally, wherein the drug in the drug delivery system is conjugated to the phospholipid via a spacer or a linker, optionally, wherein the linker comprises an aminocaproic residue.

43.-46. (canceled)
